(19) United States
(12) Patent Application Publication
(10) Pub. No.: US 2024/0112798 A1
(43) Pub. Date: Apr. 4, 2024

(54) SURGICAL SYSTEMS WITH SYNCHRONIZED DISTRIBUTED PROCESSING CAPABILITIES

(71) Applicant: Cilag GmbH International, Zug (CH)

(72) Inventors: Frederick E. Shelton, IV, Hillsboro, OH (US); Nicholas J. Ross, Franklin, OH (US); Shane R. Adams, Lebanon, OH (US); Jason L. Harris, Lebanon, OH (US)

(21) Appl. No.: 17/957,995

(22) Filed: Sep. 30, 2022

Related U.S. Application Data
(60) Provisional application No. 63/411,445, filed on Sep. 29, 2022.

(51) Int. Cl. G16H 40/63

(52) U.S. Cl. CPC G16H 40/63; A61B 2017/00221

(57) ABSTRACT

Disclosed is a surgical system for use with a surgical device. The surgical system comprises a remote processing device comprising a device control circuit and a surgical hub configured to communicably couple to the remote processing device and the surgical device. The surgical hub comprises a hub control circuit, wherein the hub control circuit and the device control circuit perform distributed processing. The surgical hub control circuit is configured to transmit a synchronization feature to the remote processing device, transmit a first subset of data associated with the surgical device to the remote processing device, perform a second analysis on a second subset of the data, determine a second result based on the second analysis, receive a first result from the remote processing device and synchronization data of the first result, assess a synchronicity of the first result and the second result based on the synchronization data.

### CROSS-REFERENCE TO RELATED APPLICATIONS

This application claims the benefit under 35 U.S.C. § 119(e) to U.S. Provisional Patent Application Ser. No. 63/411,445, titled METHOD FOR CONTROLLING SURGICAL SYSTEM DURING TISSUE TREATMENT MOTION, filed Sep. 29, 2022, the disclosure of which is herein incorporated by reference in its entirety.

### BACKGROUND

The present invention relates to surgical instruments and, in various arrangements, to surgical stapling and cutting instruments and staple cartridges for use therewith that are designed to staple and cut tissue.

### BRIEF DESCRIPTION OF THE DRAWINGS

Various features of the embodiments described herein, together with advantages thereof, may be understood in accordance with the following description taken in conjunction with the accompanying drawings as follows:

FIG. 1 is a perspective view of a powered surgical stapling system;

FIG. 2 is a perspective view of an interchangeable surgical shaft assembly of the powered surgical stapling system of FIG. 1;

FIG. 3 is an exploded assembly view of portions of a handle assembly of the powered surgical stapling system of FIG. 1;

FIG. 4 is an exploded assembly view of the interchangeable surgical shaft assembly of FIG. 2;

FIG. 5 is another partial exploded assembly view of a portion of the interchangeable surgical shaft assembly of FIG. 4;

FIG. 6 is a perspective view of a shaft assembly in accordance with at least one embodiment;

FIG. 7 is an exploded view of a distal end of the shaft assembly of FIG. 6;

FIG. 8 is a perspective view of a surgical instrument assembly comprising a proximal control interface, a shaft assembly, and an end effector assembly;

FIG. 9 is a bottom perspective view of the surgical instrument assembly of FIG. 8;

FIG. 10 is a perspective view of an example of one form of robotic controller according to one aspect of this disclosure;

FIG. 11 is a perspective view of an example of one form of robotic surgical arm cart/manipulator of a robotic surgical system operably supporting a plurality of surgical tools according to one aspect of this disclosure;

FIG. 12 is a side view of the robotic surgical arm cart/manipulator depicted in FIG. 11 according to one aspect of this disclosure;

FIG. 13 illustrates a block diagram of a surgical system for use with one or more surgical instruments, tools, and/or robotic systems in accordance with one or more aspects of the present disclosure;

FIG. 14 illustrates a block diagram of a surgical system for use with one or more surgical instruments, tools, and/or robotic systems in accordance with one or more aspects of the present disclosure;

FIG. 15 is a diagram depicting a distributed processing system, according to at least one aspect of the present disclosure.

FIG. 16 is a diagram depicting a distributed processing system, according to at least one aspect of the present disclosure.

FIG. 17 is a diagram depicting different devices and inputs accessible by a control circuit in a surgical suite, according to at least one aspect of the present disclosure.

FIG. 18 is a diagram depicting a distributed processing system, according to at least one aspect of the present disclosure.

FIG. 19 is a flow diagram depicting a distributed processing method that can be executed by a control circuit, according to at least one aspect of the present disclosure.

FIG. 20 is a flow diagram depicting a distributed processing method that can be executed by a control circuit, according to at least one aspect of the present disclosure.

FIG. 21 is a flow diagram depicting a distributed processing method that can be executed by a control circuit, according to at least one aspect of the present disclosure.

FIG. 22 is a flow diagram depicting a method for a control circuit to execute when results of a distributed processing method are out of synchronization, according to at least one aspect of the present disclosure.

FIG. 23 is a flow diagram depicting a method for a control circuit to execute when results of a distributed processing method are out of synchronization, according to at least one aspect of the present disclosure.

FIG. 24 is a flow diagram depicting a method for a control circuit to execute when results of a distributed processing method are out of synchronization, according to at least one aspect of the present disclosure.

FIG. 25 is a flow diagram depicting a method for a control circuit to execute when results of a distributed processing method are out of synchronization, according to at least one aspect of the present disclosure.

FIG. 26 is a flow diagram depicting a method for a control circuit to execute to synchronize with another control circuit, according to at least one aspect of the present disclosure.

FIG. 27 is a flow diagram depicting a method that can be executed by a control circuit to characterize the latency of another synchronized control circuit, according to at least one aspect of the present disclosure.

FIG. 28 is a flow diagram depicting a method 12190 that can be executed by a control circuit to characterize the latency of another synchronized control circuit, according to at least one aspect of the present disclosure.

FIG. 29 is a diagram depicting a surgical device with multiple subsystems, according to at least one aspect of the present disclosure.

FIG. 30 is a flow diagram depicting a prioritization method that can be executed by a control circuit for processor tasks based on a tiered matrix, according to at least one aspect of the present disclosure.

Corresponding reference characters indicate corresponding parts throughout the several views. The exemplifications set out herein illustrate various embodiments of the invention, in one form, and such exemplifications are not to be construed as limiting the scope of the invention in any manner.

### DETAILED DESCRIPTION

Applicant of the present application owns the following U.S. patent applications that were filed on even date herewith and which are each herein incorporated by reference in their respective entireties:
U.S. patent application, titled METHOD FOR CONTROLLING SURGICAL SYSTEM DURING TISSUE TREATMENT MOTION; Attorney Docket No. END9440USNP1/220061-1M;
U.S. patent application, titled ADAPTING TISSUE TREATMENT MOTION PARAMETERS BASED ON SITUATIONAL PARAMETERS; Attorney Docket No. END9440USNP2/220061-2;
U.S. patent application, titled ADAPTIVE FIRING CONTROL ALGORITHM BASED ON MECHANICAL ACTUATION OF USER CONTROLS; Attorney Docket No. END9440USNP3/220061-3;
U.S. patent application, titled ADAPTATION OF INDEPENDENT FIRING AND CLOSURE POWERED STAPLING SYSTEMS; Attorney Docket No. END9440USNP4/220061-4;
U.S. patent application, titled MONITORING ONE DRIVE SYSTEM TO ADAPT THE MOTOR DRIVEN ASPECT OF A SECOND DRIVE SYSTEM; Attorney Docket No. END9440USNP5/220061-5;
U.S. patent application, titled ADJUSTMENT OF THE MOTOR CONTROL PROGRAM BASED ON DETECTION OF INDIVIDUAL DEVICE DRIVE TRAIN PROPERTIES; Attorney Docket No. END9440USNP6/220061-6;
U.S. patent application, titled ADJUSTMENT OF A MOTOR CONTROL COMMAND SIGNAL TO ADAPT TO SYSTEM CHANGES; Attorney Docket No. END9440USNP7/220061-7;
U.S. patent application, titled MOTOR ADJUSTMENTS IN ABSENCE OF MOTOR DRIVE SIGNAL; Attorney Docket No. END9440USNP8/220061-8;
U.S. patent application, titled SURGICAL SYSTEM WITH MOTOR RELATIVE CAPACITY INTERROGATIONS; Attorney Docket No. END9440USNP10/220061-10;
U.S. patent application, titled MOTOR CONTROL OF SURGICAL INSTRUMENT SYSTEMS; Attorney Docket No. END9440USNP11/220061-11;
U.S. patent application, titled SURGICAL SYSTEM WITH AMPLITUDE AND PULSE WIDTH MODULATION ADJUSTMENTS; Attorney Docket No. END9440USNP12/220061-12;
U.S. patent application, titled SURGICAL ALGORITHMS WITH INCREMENTAL SENSORY ACTIONS; Attorney Docket No. END9440USNP13/220061-13;
U.S. patent application, titled UTILIZING LOCAL FIRING PARAMETERS TO INITIATE MOTOR CONTROL ADJUSTMENTS IN SURGICAL SYSTEMS; Attorney Docket No. END9440USNP14/220061-14; and
U.S. patent application, titled SURGICAL SYSTEMS WITH DYNAMIC FORCE TO FIRE ADJUSTMENTS; Attorney Docket No. END9440USNP15/220061-15.

Numerous specific details are set forth to provide a thorough understanding of the overall structure, function, manufacture, and use of the embodiments as described in the specification and illustrated in the accompanying drawings. Well-known operations, components, and elements have not been described in detail so as not to obscure the embodiments described in the specification. The reader will understand that the embodiments described and illustrated herein are non-limiting examples, and thus it can be appreciated that the specific structural and functional details disclosed herein may be representative and illustrative. Variations and changes thereto may be made without departing from the scope of the claims.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a surgical system, device, or apparatus that “comprises,” “has,” “includes” or “contains” one or more elements possesses those one or more elements, but is not limited to possessing only those one or more elements. Likewise, an element of a system, device, or apparatus that “comprises,” “has,” “includes” or “contains” one or more features possesses those one or more features, but is not limited to possessing only those one or more features.

The terms “proximal” and “distal” are used herein with reference to a clinician manipulating the handle portion of the surgical instrument. The term “proximal” refers to the portion closest to the clinician and the term “distal” refers to the portion located away from the clinician. It will be further appreciated that, for convenience and clarity, spatial terms such as “vertical”, “horizontal”, “up”, and “down” may be used herein with respect to the drawings. However, surgical instruments are used in many orientations and positions, and these terms are not intended to be limiting and/or absolute.

Various exemplary devices and methods are provided for performing laparoscopic and minimally invasive surgical procedures. However, the reader will readily appreciate that the various methods and devices disclosed herein can be used in numerous surgical procedures and applications including, for example, in connection with open surgical procedures. As the present Detailed Description proceeds, the reader will further appreciate that the various instruments disclosed herein can be inserted into a body in any way, such as through a natural orifice, through an incision or puncture hole formed in tissue, etc. The working portions or end effector portions of the instruments can be inserted directly into a patient's body or can be inserted through an access device that has a working channel through which the end effector and elongate shaft of a surgical instrument can be advanced.

A surgical stapling system can comprise a shaft and an end effector extending from the shaft. The end effector comprises a first jaw and a second jaw. The first jaw comprises a staple cartridge. The staple cartridge is insertable into and removable from the first jaw; however, other embodiments are envisioned in which a staple cartridge is not removable from, or at least readily replaceable from, the first jaw. The second jaw comprises an anvil configured to deform staples ejected from the staple cartridge. The second jaw is pivotable relative to the first jaw about a closure axis; however, other embodiments are envisioned in which the first jaw is pivotable relative to the second jaw. The surgical stapling system further comprises an articulation joint configured to permit the end effector to be rotated, or articulated, relative to the shaft. The end effector is rotatable about an articulation axis extending through the articulation joint. Other embodiments are envisioned which do not include an articulation joint.

The staple cartridge comprises a cartridge body. The cartridge body includes a proximal end, a distal end, and a deck extending between the proximal end and the distal end. In use, the staple cartridge is positioned on a first side of the tissue to be stapled and the anvil is positioned on a second side of the tissue. The anvil is moved toward the staple cartridge to compress and clamp the tissue against the deck. Thereafter, staples removably stored in the cartridge body can be deployed into the tissue. The cartridge body includes staple cavities defined therein wherein staples are removably stored in the staple cavities. The staple cavities are arranged in six longitudinal rows. Three rows of staple cavities are positioned on a first side of a longitudinal slot and three rows of staple cavities are positioned on a second side of the longitudinal slot. Other arrangements of staple cavities and staples may be possible.

The staples are supported by staple drivers in the cartridge body. The drivers are movable between a first, or unfired position, and a second, or fired, position to eject the staples from the staple cavities. The drivers are retained in the cartridge body by a retainer which extends around the bottom of the cartridge body and includes resilient members configured to grip the cartridge body and hold the retainer to the cartridge body. The drivers are movable between their unfired positions and their fired positions by a sled. The sled is movable between a proximal position adjacent the proximal end and a distal position adjacent the distal end. The sled comprises a plurality of ramped surfaces configured to slide under the drivers and lift the drivers, and the staples supported thereon, toward the anvil.

Further to the above, the sled is moved distally by a firing member. The firing member is configured to contact the sled and push the sled toward the distal end. The longitudinal slot defined in the cartridge body is configured to receive the firing member. The anvil also includes a slot configured to receive the firing member. The firing member further comprises a first cam which engages the first jaw and a second cam which engages the second jaw. As the firing member is advanced distally, the first cam and the second cam can control the distance, or tissue gap, between the deck of the staple cartridge and the anvil. The firing member also comprises a knife configured to incise the tissue captured intermediate the staple cartridge and the anvil. It is desirable for the knife to be positioned at least partially proximal to the ramped surfaces such that the staples are ejected ahead of the knife,

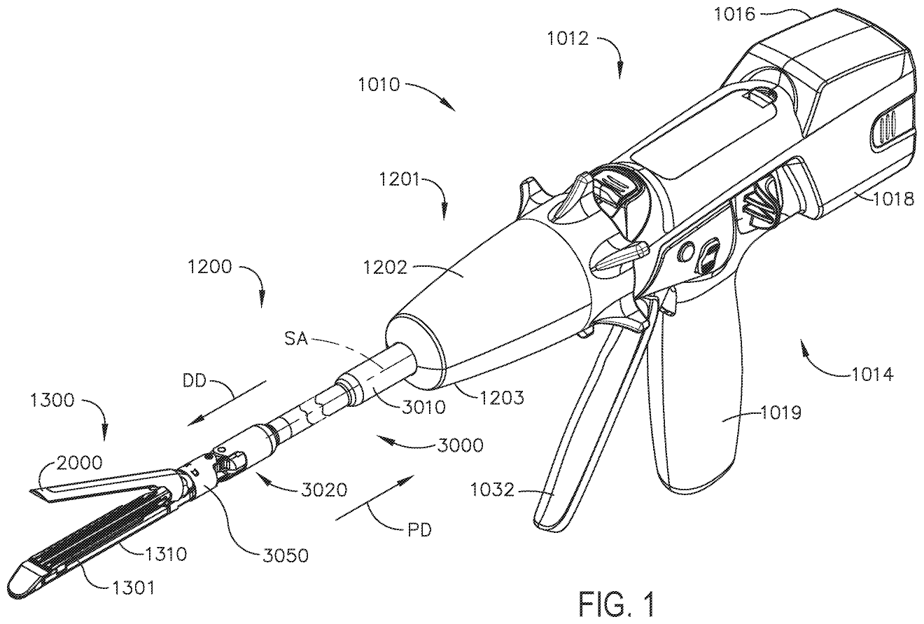

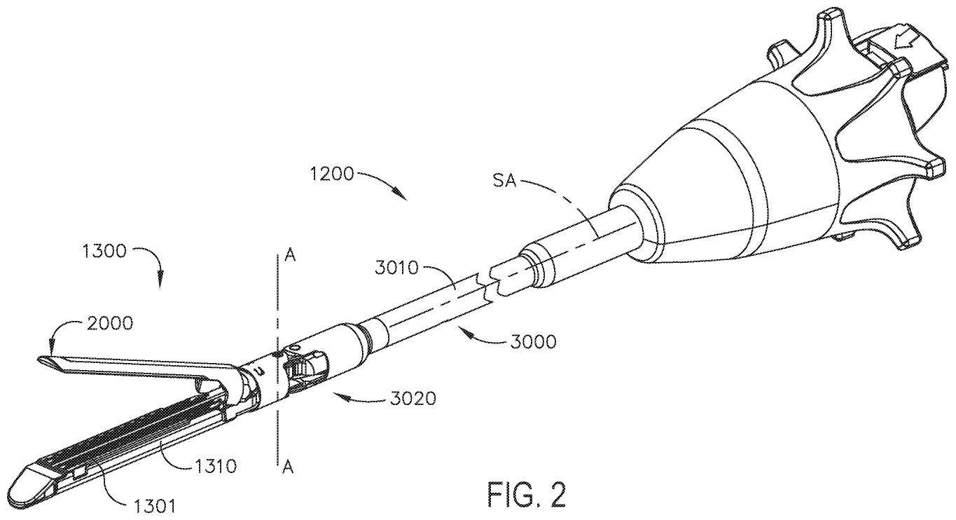

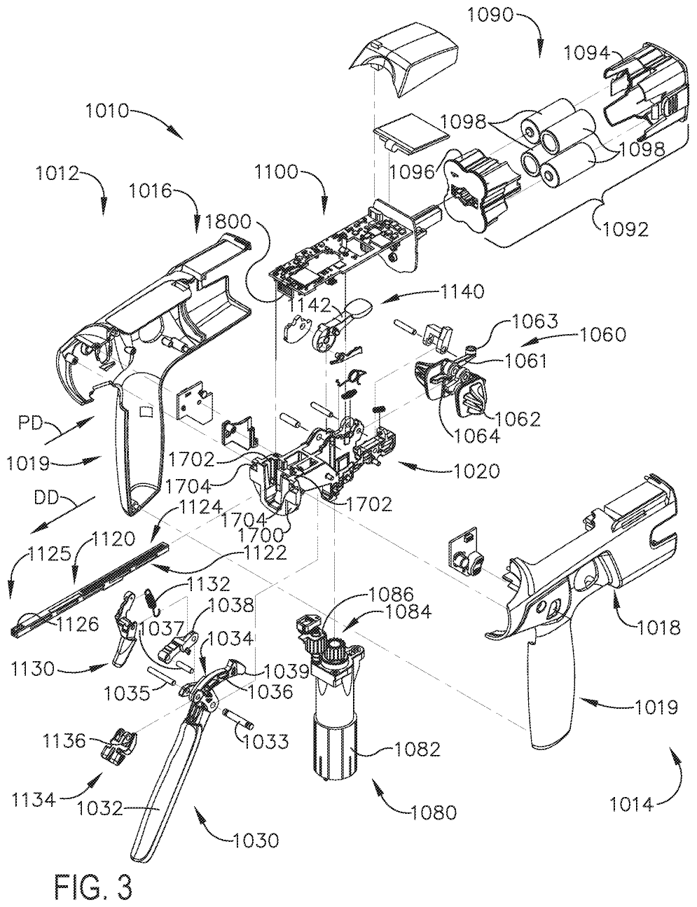

FIG. 1 illustrates the surgical instrument 1010 that includes an interchangeable shaft assembly 1200 operably coupled to a housing 1012. FIG. 2 illustrates the interchangeable shaft assembly 1200 detached from the housing 1012 or handle 1014. As can be seen in FIG. 3, the handle 1014 may comprise a pair of interconnectable handle housing segments 1016 and 1018 that may be interconnected by screws, snap features, adhesive, etc. In the illustrated arrangement, the handle housing segments 1016, 1018 cooperate to form a pistol grip portion 1019. FIGS. 1 and 3 depict a motor-driven surgical cutting and fastening instrument 1010 that may or may not be reused. In the illustrated embodiment, the instrument 1010 includes a proximal housing 1012 that comprises a handle 1014 that is configured to be grasped, manipulated and actuated by the clinician. The housing 1012 is configured for operable attachment to an interchangeable shaft assembly 1200 that has a surgical end effector 1300 operably coupled thereto that is configured to perform one or more surgical tasks or procedures. As the present Detailed Description proceeds, it will be understood that the various forms of interchangeable shaft assemblies disclosed herein may also be effectively employed in connection with robotically-controlled surgical systems. Thus, the term “housing” may also encompass a housing or similar portion of a robotic system that houses or otherwise operably supports at least one drive system that is configured to generate and apply at least one control motion which could be used to actuate the interchangeable shaft assemblies disclosed herein and their respective equivalents. In addition, various components may be “housed” or contained in the housing or various components may be “associated with” a housing. In such instances, the components may not be contained within the housing or supported directly by the housing. The term “frame” may refer to a portion of a handheld surgical instrument. The term “frame” may also represent a portion of a robotically controlled surgical instrument and/or a portion of the robotic system that may be used to operably control a surgical instrument. For example, the interchangeable shaft assemblies disclosed herein may be employed with various robotic systems, instruments, components and methods disclosed in U.S. Pat. No. 9,072,535, entitled SURGICAL STAPLING INSTRUMENTS WITH ROTATABLE STAPLE DEPLOYMENT ARRANGEMENTS, that is incorporated by reference herein in its entirety.

The proximal housing 1012 depicted in FIG. 1 is shown in connection with an interchangeable shaft assembly 1200 (FIGS. 2, 4 and 5) that includes an end effector 1300 that comprises a surgical cutting and fastening device that is configured to operably support a surgical staple cartridge 1301 therein. The housing 1012 may be configured for use in connection with interchangeable shaft assemblies that include end effectors that are adapted to support different sizes and types of staple cartridges, have different shaft lengths, sizes, and types, etc. In addition, the housing 1012 may also be effectively employed with a variety of other interchangeable shaft assemblies including those assemblies that are configured to apply other motions and forms of energy such as, for example, radio frequency (RF) energy, ultrasonic energy and/or motion to end effector arrangements adapted for use in connection with various surgical applications and procedures. Furthermore, the end effectors, shaft assemblies, handles, surgical instruments, and/or surgical instrument systems can utilize any suitable fastener that can be gripped and manipulated by the clinician. As will be discussed in further detail below, the handle 1014 operably supports a plurality of drive systems therein that are configured to generate and apply various control motions to corresponding portions of the interchangeable shaft assembly that is operably attached thereto.

Referring now to FIG. 3, the handle 1014 may further include a frame 1020 that operably supports a plurality of drive systems. For example, the frame 1020 can operably support a “first” or closure drive system, generally designated as 1030, which may be employed to apply closing and opening motions to the interchangeable shaft assembly 1200 that is operably attached or coupled thereto. In at least one form, the closure drive system 1030 may include an actuator in the form of a closure trigger 1032 that is pivotally supported by the frame 1020. More specifically, as illustrated in FIG. 3, the closure trigger 1032 is pivotally coupled to the handle 1014 by a pin 1033. Such arrangement enables the closure trigger 1032 to be manipulated by a clinician such that when the clinician grips the pistol grip portion 1019 of the handle 1014, the closure trigger 1032 may be easily pivoted from a starting or “unactuated” position to an “actuated” position and more particularly to a fully compressed or fully actuated position. The closure trigger 1032 may be biased into the unactuated position by spring or other biasing arrangement (not shown). In various forms, the closure drive system 1030 further includes a closure linkage assembly 1034 that is pivotally coupled to the closure trigger 1032. As can be seen in FIG. 3, the closure linkage assembly 1034 may include a first closure link 1036 and a second closure link 1038 that are pivotally coupled to the closure trigger 1032 by a pin 1035. The second closure link 1038 may also be referred to herein as an “attachment member” and include a transverse attachment pin 1037.

Still referring to FIG. 3, it can be observed that the first closure link 1036 may have a locking wall or end 1039 thereon that is configured to cooperate with a closure release assembly 1060 that is pivotally coupled to the frame 1020. In at least one form, the closure release assembly 1060 may comprise a release button assembly 1062 that has a distally protruding locking pawl 1064 formed thereon. The release button assembly 1062 may be pivoted in a counterclockwise direction by a release spring (not shown). As the clinician depresses the closure trigger 1032 from its unactuated position towards the pistol grip portion 1019 of the handle 1014, the first closure link 1036 pivots upward to a point wherein the locking pawl 1064 drops into retaining engagement with the locking wall 1039 on the first closure link 1036 thereby preventing the closure trigger 1032 from returning to the unactuated position. Thus, the closure release assembly 1060 serves to lock the closure trigger 1032 in the fully actuated position. When the clinician desires to unlock the closure trigger 1032 to permit it to be biased to the unactuated position, the clinician simply pivots the closure release button assembly 1062 such that the locking pawl 1064 is moved out of engagement with the locking wall 1039 on the first closure link 1036. When the locking pawl 1064 has been moved out of engagement with the first closure link 1036, the closure trigger 1032 may pivot back to the unactuated position. Other closure trigger locking and release arrangements may also be employed.

An arm 1061 may extend from the closure release button assembly 1062. A magnetic element 1063, such as a permanent magnet, for example, may be mounted to the arm 1061. When the closure release button assembly 1062 is rotated from its first position to its second position, the magnetic element 1063 can move toward a circuit board 1100. The circuit board 1100 can include at least one sensor that is configured to detect the movement of the magnetic element 1063. In at least one embodiment, for example, a “Hall Effect” sensor (not shown) can be mounted to the bottom surface of the circuit board 1100. The Hall Effect sensor can be configured to detect changes in a magnetic field surrounding the Hall Effect sensor caused by the movement of the magnetic element 1063. The Hall Effect sensor can be in signal communication with a microcontroller, for example, which can determine whether the closure release button assembly 1062 is in its first position, which is associated with the unactuated position of the closure trigger 1032 and the open configuration of the end effector, its second position, which is associated with the actuated position of the closure trigger 1032 and the closed configuration of the end effector, and/or any position between the first position and the second position.

In at least one form, the handle 1014 and the frame 1020 may operably support another drive system referred to herein as a firing drive system 1080 that is configured to apply firing motions to corresponding portions of the interchangeable shaft assembly attached thereto. The firing drive system 1080 may also be referred to herein as a “second drive system”. The firing drive system 1080 may employ an electric motor 1082 that is located in the pistol grip portion 1019 of the handle 1014. In various forms, the motor 1082 may be a DC brushed driving motor having a maximum rotation of, approximately, 25,000 RPM, for example. In other arrangements, the motor may include a brushless motor, a cordless motor, a synchronous motor, a stepper motor, or any other suitable electric motor. The motor 1082 may be powered by a power source 1090 that in one form may comprise a removable power pack 1092. As can be seen in FIG. 3, for example, the power pack 1092 may comprise a proximal housing portion 1094 that is configured for attachment to a distal housing portion 1096. The proximal housing portion 1094 and the distal housing portion 1096 are configured to operably support a plurality of batteries 1098 therein. Batteries 1098 may each comprise, for example, a Lithium Ion (“LI”) or other suitable battery. The distal housing portion 1096 is configured for removable operable attachment to the circuit board 1100 which is also operably coupled to the motor 1082. A number of batteries 1098 may be connected in series may be used as the power source for the surgical instrument 1010. In addition, the power source 1090 may be replaceable and/or rechargeable.

As outlined above with respect to other various forms, the electric motor 1082 can include a rotatable shaft (not shown) that operably interfaces with a gear reducer assembly 1084 that is mounted in meshing engagement with a set, or rack, of drive teeth 1122 on a longitudinally movable drive member 1120. In use, a voltage polarity provided by the power source 1090 can operate the electric motor 1082 in a clockwise direction wherein the voltage polarity applied to the electric motor by the battery can be reversed in order to operate the electric motor 1082 in a counter-clockwise direction. When the electric motor 1082 is rotated in one direction, the drive member 1120 will be axially driven in the distal direction “DD”. When the motor 1082 is driven in the opposite rotary direction, the drive member 1120 will be axially driven in a proximal direction “PD”. The handle 1014 can include a switch which can be configured to reverse the polarity applied to the electric motor 1082 by the power source 1090. As with the other forms described herein, the handle 1014 can also include a sensor that is configured to detect the position of the drive member 1120 and/or the direction in which the drive member 1120 is being moved.

Actuation of the motor 1082 can be controlled by a firing trigger 1130 that is pivotally supported on the handle 1014. The firing trigger 1130 may be pivoted between an unactuated position and an actuated position. The firing trigger 1130 may be biased into the unactuated position by a spring 1132 or other biasing arrangement such that when the clinician releases the firing trigger 1130, it may be pivoted or otherwise returned to the unactuated position by the spring 1132 or biasing arrangement. In at least one form, the firing trigger 1130 can be positioned “outboard” of the closure trigger 1032 as was discussed above. In at least one form, a firing trigger safety button 1134 may be pivotally mounted to the closure trigger 1032 by the pin 1035. The safety button 1134 may be positioned between the firing trigger 1130 and the closure trigger 1032 and have a pivot arm 1136 protruding therefrom. When the closure trigger 1032 is in the unactuated position, the safety button 1134 is contained in the handle 1014 where the clinician cannot readily access it and move it between a safety position preventing actuation of the firing trigger 1130 and a firing position wherein the firing trigger 1130 may be fired. As the clinician depresses the closure trigger 1032, the safety button 1134 and the firing trigger 1130 pivot down wherein they can then be manipulated by the clinician.

As indicated above, in at least one form, the longitudinally movable drive member 1120 has a rack of teeth 1122 formed thereon for meshing engagement with a corresponding drive gear 1086 of the gear reducer assembly 1084. At least one form also includes a manually-actuatable “bailout” assembly 1140 that is configured to enable the clinician to manually retract the longitudinally movable drive member 1120 should the motor 1082 become disabled. The bailout assembly 1140 may include a lever or bailout handle assembly 1142 that is configured to be manually pivoted into ratcheting engagement with teeth 1124 also provided in the drive member 1120. Thus, the clinician can manually retract the drive member 1120 by using the bailout handle assembly 1142 to ratchet the drive member 1120 in the proximal direction “PD”. U.S. Pat. No. 8,608,045, entitled POWERED SURGICAL CUTTING AND STAPLING APPARATUS WITH MANUALLY RETRACTABLE FIRING SYSTEM, discloses bailout arrangements and other components, arrangements and systems that may also be employed with the various instruments disclosed herein. U.S. Pat. No. 8,608,045, is hereby incorporated by reference herein in its entirety.

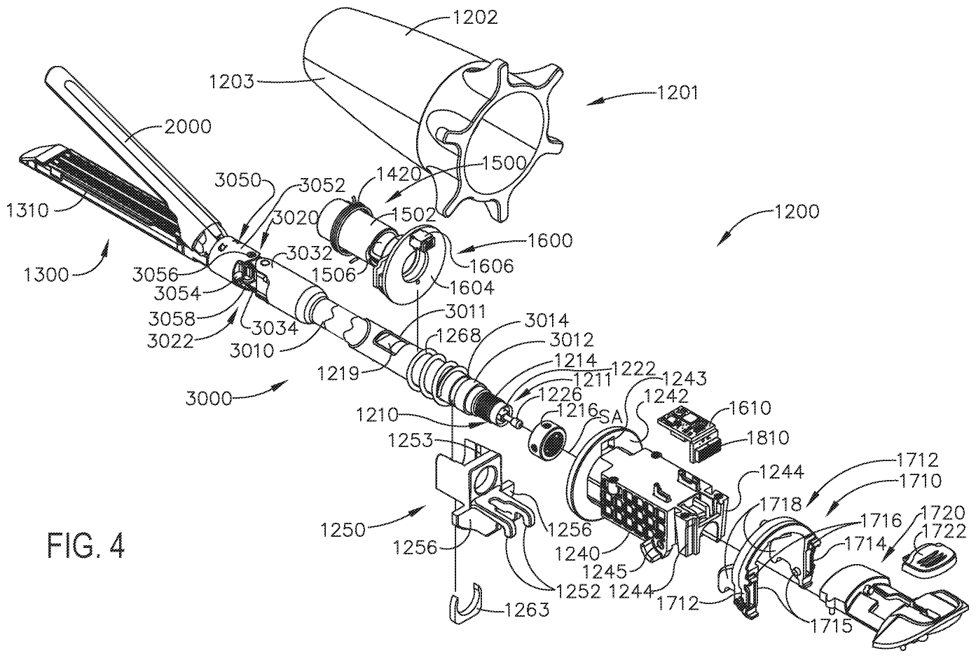

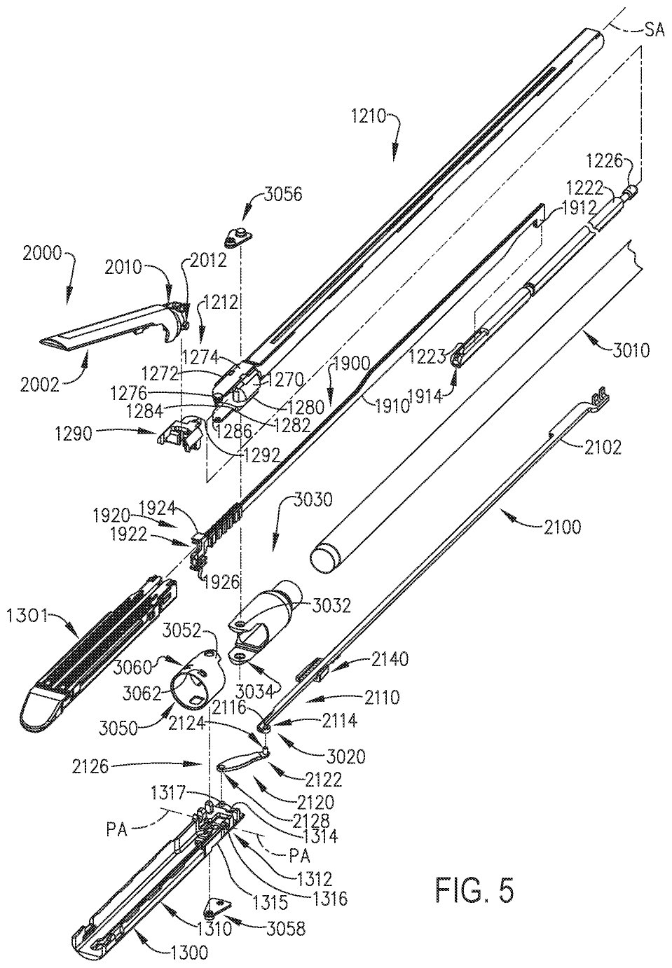

Turning now to FIGS. 2 and 5, the interchangeable shaft assembly 1200 includes a surgical end effector 1300 that comprises an elongate channel 1310 that is configured to operably support a staple cartridge 1301 therein. The end effector 1300 may further include an anvil 2000 that is pivotally supported relative to the elongate channel 1310. The interchangeable shaft assembly 1200 may further include an articulation joint 3020 and an articulation lock 2140 which can be configured to releasably hold the end effector 1300 in a desired position relative to a shaft axis SA. Examples of various features of at least one form of the end effector 1300, the articulation joint 3020 and articulation locks may be found in U.S. patent application Ser. No. 13/803,086, filed Mar. 14, 2013, entitled ARTICULATABLE SURGICAL INSTRUMENT COMPRISING AN ARTICULATION LOCK, now U.S. Patent Application Publication No. 2014/0263541. The entire disclosure of U.S. patent application Ser. No. 13/803,086, filed Mar. 14, 2013, entitled ARTICULATABLE SURGICAL INSTRUMENT COMPRISING AN ARTICULATION LOCK, now U.S. Patent Application Publication No. 2014/0263541, is hereby incorporated by reference herein. As can be seen in FIG. 4, the interchangeable shaft assembly 1200 can further include a proximal housing or nozzle 1201 comprised of nozzle portions 1202 and 1203.

The interchangeable shaft assembly 1200 can further include a closure system or closure member assembly 3000 which can be utilized to close and/or open the anvil 2000 of the end effector 1300. The shaft assembly 1200 can include a spine 1210 that is configured to, one, slidably support a firing member therein and, two, slidably support the closure member assembly 3000 which extends around the spine 1210. As can be seen in FIG. 5, a distal end 1212 of spine 1210 terminates in an upper lug mount feature 1270 and in a lower lug mount feature 1280. The upper lug mount feature 1270 is formed with a lug slot 1272 therein that is adapted to mountingly support an upper mounting link 1274 therein. Similarly, the lower lug mount feature 1280 is formed with a lug slot 1282 therein that is adapted to mountingly support a lower mounting link 1284 therein. The upper mounting link 1274 includes a pivot socket 1276 therein that is adapted to rotatably receive therein a pivot pin 1292 that is formed on a channel cap or anvil retainer 1290 that is attached to a proximal end portion 1312 of the elongate channel 1310. The lower mounting link 1284 includes lower pivot pin 1286 that adapted to be received within a pivot hole 1314 formed in the proximal end portion 1312 of the elongate channel 1310. See FIG. 5. The lower pivot pin 1286 is vertically aligned with the pivot socket 1276 to define an articulation axis AA about which the surgical end effector 1300 may articulate relative to the shaft axis SA. See FIG. 2.

In the illustrated example, the surgical end effector 1300 is selectively articulatable about the articulation axis AA by an articulation system 2100. In one form, the articulation system 2100 includes proximal articulation driver 2102 that is pivotally coupled to an articulation link 2120. As can be most particularly seen in FIG. 5, an offset attachment lug 2114 is formed on a distal end 2110 of the proximal articulation driver 2102. A pivot hole 2116 is formed in the offset attachment lug 2114 and is configured to pivotally receive therein a proximal link pin 2124 formed on the proximal end 2122 of the articulation link 2120. A distal end 2126 of the articulation link 2120 includes a pivot hole 2128 that is configured to pivotally receive therein a channel pin 1317 formed on the proximal end portion 1312 of the elongate channel 1310. Thus, axial movement of proximal articulation driver 2102 will thereby apply articulation motions to the elongate channel 1310 to thereby cause the surgical end effector 1300 to articulate about the articulation axis AA relative to the spine 1210. Further details concerning the construction and operation of the articulation system 2100 may be found in various references incorporated by reference herein including U.S. patent application Ser. No. 15/635,631, filed Jun. 28, 2017, entitled SURGICAL INSTRUMENT WITH AXIALLY MOVABLE CLOSURE MEMBER, now U.S. Patent Application Publication No. 2019/0000464, the entire disclosure of which is hereby incorporated by reference herein. In various circumstances, the proximal articulation driver 2102 can be held in position by an articulation lock 2140 when the proximal articulation driver 2102 is not being moved in the proximal or distal directions. Additional details regarding an example of an articulation lock 2140 may be found in U.S. patent application Ser. No. 15/635,631, now U.S. Patent Application Publication No. 2019/0000464, as well as in other references incorporated by reference herein.

In various circumstances, the spine 1210 can comprise a proximal end 1211 which is rotatably supported in a chassis 1240. In one arrangement, for example, the proximal end 1211 of the spine 1210 has a thread 1214 formed thereon for threaded attachment to a spine bearing 1216 configured to be supported within the chassis 1240. See FIG. 4. Such an arrangement facilitates rotatable attachment of the spine 1210 to the chassis 1240 such that the spine 1210 may be selectively rotated about a shaft axis SA relative to the chassis 1240.

Referring primarily to FIG. 4, the interchangeable shaft assembly 1200 includes a closure shuttle 1250 that is slidably supported within the chassis 1240 such that it may be axially moved relative thereto. The closure shuttle 1250 includes a pair of proximally-protruding hooks 1252 that are configured for attachment to the attachment pin 1037 (FIG. 3) that is attached to the second closure link 1038 as will be discussed in further detail below. In at least one example, the closure member assembly 3000 comprises a proximal closure member segment 3010 that has a proximal end 3012 that is coupled to the closure shuttle 1250 for relative rotation thereto. For example, a U shaped connector 1263 is inserted into an annular slot 3014 in the proximal end 3012 of the proximal closure member segment 3010 and is retained within vertical slots 1253 in the closure shuttle 1250. Such an arrangement serves to attach the proximal closure member segment 3010 to the closure shuttle 1250 for axial travel therewith while enabling the proximal closure member segment 3010 to rotate relative to the closure shuttle 1250 about the shaft axis SA. A closure spring 1268 is journaled on the proximal closure member segment 3010 and serves to bias the proximal closure member segment 3010 in the proximal direction “PD” which can serve to pivot the closure trigger 1032 into the unactuated position when the shaft assembly is operably coupled to the handle 1014.

In at least one form, the interchangeable shaft assembly 1200 may further include an articulation joint 3020. Other interchangeable shaft assemblies, however, may not be capable of articulation. As can be seen in FIG. 5, for example, a distal closure member or distal closure tube segment 3030 is coupled to the distal end of the proximal closure member segment 3010. The articulation joint 3020 includes a double pivot closure sleeve assembly 3022. According to various forms, the double pivot closure sleeve assembly 3022 includes an end effector closure tube 3050 having upper and lower distally projecting tangs 3052, 3054. An upper double pivot link 3056 includes upwardly projecting distal and proximal pivot pins that engage respectively an upper distal pin hole in the upper proximally projecting tang 3052 and an upper proximal pin hole in an upper distally projecting tang 3032 on the distal closure tube segment 3030. A lower double pivot link 3058 includes upwardly projecting distal and proximal pivot pins that engage respectively a lower distal pin hole in the lower proximally projecting tang 3054 and a lower proximal pin hole in the lower distally projecting tang 3034. See FIGS. 4 and 5. As will be discussed in further detail below, the closure member assembly 3000 is translated distally (direction “DD”) to close the anvil 2000, for example, in response to the actuation of the closure trigger 1032. The anvil 2000 is opened by proximally translating the closure member assembly 3000 which causes the end effector closure sleeve to interact with the anvil 2000 and pivot it to an open position.

As was also indicated above, the interchangeable shaft assembly 1200 further includes a firing member 1900 that is supported for axial travel within the spine 1210. The firing member 1900 includes an intermediate firing shaft portion 1222 that is configured for attachment to a distal cutting portion or knife bar 1910. The intermediate firing shaft portion 1222 may include a longitudinal slot 1223 in the distal end thereof which can be configured to receive a tab 1912 on the proximal end of the distal knife bar 1910. The longitudinal slot 1223 and the proximal end tab 1912 can be sized and configured to permit relative movement therebetween and can comprise a slip joint 1914. The slip joint 1914 can permit the intermediate firing shaft portion 1222 of the firing member 1900 to be moved to articulate the end effector 1300 without moving, or at least substantially moving, the knife bar 1910. Once the end effector 1300 has been suitably oriented, the intermediate firing shaft portion 1222 can be advanced distally until a proximal sidewall of the longitudinal slot 1223 comes into contact with the tab 1912 in order to advance the knife bar 1910 and fire the staple cartridge 1301 positioned within the channel 1310. The knife bar 1910 includes a knife portion 1920 that includes a blade or tissue cutting edge 1922 and includes an upper anvil engagement tab 1924 and lower channel engagement tabs 1926. Various firing member configurations and operations are disclosed in various other references incorporated herein by reference.

Embodiments are also envisioned where, in lieu of a slip joint 1914, a shifter assembly can be used. Details of such a shifter assembly and corresponding components, assemblies, and systems can be found in U.S. patent application Ser. No. 15/635,521, entitled SURGICAL INSTRUMENT LOCKOUT ARRANGEMENT, which is incorporated by reference herein in its entirety.

As can be seen in FIG. 4, the shaft assembly 1200 further includes a switch drum 1500 that is rotatably received on proximal closure member segment 3010. The switch drum 1500 comprises a hollow shaft segment 1502 that has a shaft boss formed thereon for receiving an outwardly protruding actuation pin therein. In various circumstances, the actuation pin extends through a longitudinal slot provided in the lock sleeve to facilitate axial movement of the lock sleeve when it is engaged with the articulation driver. A rotary torsion spring 1420 is configured to engage the boss on the switch drum 1500 and a portion of the nozzle housing 1203 to apply a biasing force to the switch drum 1500. The switch drum 1500 can further comprise at least partially circumferential openings 1506 defined therein which can be configured to receive circumferential mounts extending from the nozzle portions 1202, 1203 and permit relative rotation, but not translation, between the switch drum 1500 and the nozzle 1201. The mounts also extend through openings 3011 in the proximal closure member segment 3010 to be seated in recesses 1219 in the spine 1210. Rotation of the switch drum 1500 about the shaft axis SA will ultimately result in the rotation of the actuation pin and the lock sleeve between its engaged and disengaged positions. In one arrangement, the rotation of the switch drum 1500 may be linked to the axial advancement of the closure tube or closure member. Thus, in essence, actuation of the closure system may operably engage and disengage the articulation drive system with the firing drive system in the various manners described in further detail in U.S. patent application Ser. No. 13/803,086, entitled ARTICULATABLE SURGICAL INSTRUMENT COMPRISING AN ARTICULATION LOCK, now U.S. Patent Application Publication No. 2014/0263541, and U.S. Pat. No. 9,913,642, entitled SURGICAL INSTRUMENT COMPRISING A SENSOR SYSTEM, the entire disclosures of each being hereby incorporated by reference herein. For example, when the closure tube is in its proximal-most position corresponding to a “jaws open” position, the closure member segment 3010 will have positioned the switch drum 1500 so as to link the articulation system with the firing drive system. When, the closure tube has been moved to its distal position corresponding to a “jaws closed” position, the closure tube has rotated the switch drum 1500 to a position wherein the articulation system is delinked from the firing drive system.

As also illustrated in FIG. 4, the shaft assembly 1200 can comprise a slip ring assembly 1600 which can be configured to conduct electrical power to and/or from the end effector 1300 and/or communicate signals to and/or from the end effector 1300, for example. The slip ring assembly 1600 can comprise a proximal connector flange 1604 that is mounted to a chassis flange 1242 that extends from the chassis 1240 and a distal connector flange that is positioned within a slot defined in the shaft housings. The proximal connector flange 1604 can comprise a first face and the distal connector flange can comprise a second face which is positioned adjacent to and movable relative to the first face. The distal connector flange can rotate relative to the proximal connector flange 1604 about the shaft axis SA. The proximal connector flange 1604 can comprise a plurality of concentric, or at least substantially concentric, conductors defined in the first face thereof. A connector can be mounted on the proximal side of the connector flange and may have a plurality of contacts wherein each contact corresponds to and is in electrical contact with one of the conductors. Such an arrangement permits relative rotation between the proximal connector flange 1604 and the distal connector flange while maintaining electrical contact therebetween. The proximal connector flange 1604 can include an electrical connector 1606 which can place the conductors in signal communication with a shaft circuit board 1610 mounted to the shaft chassis 1240, for example. In at least one instance, a wiring harness comprising a plurality of conductors can extend between the electrical connector 1606 and the shaft circuit board 1610. The electrical connector 1606 may extend proximally through a connector opening 1243 defined in the chassis flange 1242. See FIG. 4. Further details regarding slip ring assembly 1600 may be found in U.S. patent application Ser. No. 13/803,086, entitled ARTICULATABLE SURGICAL INSTRUMENT COMPRISING AN ARTICULATION LOCK, now U.S. Patent Application Publication No. 2014/0263541, U.S. patent application Ser. No. 13/800,067, entitled STAPLE CARTRIDGE TISSUE THICKNESS SENSOR SYSTEM, filed on Mar. 13, 2013, now U.S. Patent Application Publication No. 2014/0263552, and U.S. Pat. No. 9,345,481, entitled STAPLE CARTRIDGE TISSUE THICKNESS SENSOR SYSTEM, for example. U.S. patent application Ser. No. 13/803,086, now U.S. Patent Application Publication No. 2014/0263541, U.S. patent application Ser. No. 13/800,067, now U.S. Patent Application Publication No. 2014/0263552, and U.S. Pat. No. 9,345,481 are each hereby incorporated by reference herein in their respective entireties.

As discussed above, the shaft assembly 1200 can include a proximal portion which is fixably mounted to the handle 1014 and a distal portion which is rotatable about a longitudinal axis. The rotatable distal shaft portion can be rotated relative to the proximal portion about the slip ring assembly 1600, as discussed above. The distal connector flange of the slip ring assembly 1600 can be positioned within the rotatable distal shaft portion. Moreover, further to the above, the switch drum 1500 can also be positioned within the rotatable distal shaft portion. When the rotatable distal shaft portion is rotated, the distal connector flange and the switch drum 1500 can be rotated synchronously with one another. In addition, the switch drum 1500 can be rotated between a first position and a second position relative to the distal connector flange. When the switch drum 1500 is in its first position, the articulation drive system may be operably disengaged from the firing drive system and, thus, the operation of the firing drive system may not articulate the end effector 1300 of the shaft assembly 1200. When the switch drum 1500 is in its second position, the articulation drive system may be operably engaged with the firing drive system and, thus, the operation of the firing drive system may articulate the end effector 1300 of the shaft assembly 1200. When the switch drum 1500 is moved between its first position and its second position, the switch drum 1500 is moved relative to the distal connector flange. In various instances, the shaft assembly 1200 can comprise at least one sensor configured to detect the position of the switch drum 1500.

Referring again to FIG. 4, the chassis 1240 includes at least one, and preferably two, tapered attachment portions 1244 formed thereon that are adapted to be received within corresponding dovetail slots 1702 formed within a distal attachment flange portion 1700 of the frame 1020. See FIG. 3. Each dovetail slot 1702 may be tapered or, stated another way, be somewhat V-shaped to seatingly receive the attachment portions 1244 therein. As can be further seen in FIG. 4, a shaft attachment lug 1226 is formed on the proximal end of the intermediate firing shaft portion 1222. As will be discussed in further detail below, when the interchangeable shaft assembly 1200 is coupled to the handle 1014, the shaft attachment lug 1226 is received in a firing shaft attachment cradle 1126 formed in a distal end 1125 of the longitudinal drive member 1120. See FIG. 3.

Various shaft assembly embodiments employ a latch system 1710 for removably coupling the shaft assembly 1200 to the housing 1012 and more specifically to the frame 1020. As can be seen in FIG. 4, for example, in at least one form, the latch system 1710 includes a lock member or lock yoke 1712 that is movably coupled to the chassis 1240. In the illustrated embodiment, for example, the lock yoke 1712 has a U-shape with two spaced downwardly extending legs 1714. The legs 1714 each have a pivot lug 1715 formed thereon that are adapted to be received in corresponding holes 1245 formed in the chassis 1240. Such arrangement facilitates pivotal attachment of the lock yoke 1712 to the chassis 1240. The lock yoke 1712 may include two proximally protruding lock lugs 1716 that are configured for releasable engagement with corresponding lock detents or grooves 1704 in the distal attachment flange portion 1700 of the frame 1020. See FIG. 3. In various forms, the lock yoke 1712 is biased in the proximal direction by spring or biasing member (not shown). Actuation of the lock yoke 1712 may be accomplished by a latch button 1722 that is slidably mounted on a latch actuator assembly 1720 that is mounted to the chassis 1240. The latch button 1722 may be biased in a proximal direction relative to the lock yoke 1712. As will be discussed in further detail below, the lock yoke 1712 may be moved to an unlocked position by biasing the latch button in the distal direction which also causes the lock yoke 1712 to pivot out of retaining engagement with the distal attachment flange portion 1700 of the frame 1020. When the lock yoke 1712 is in “retaining engagement” with the distal attachment flange portion 1700 of the frame 1020, the lock lugs 1716 are retainingly seated within the corresponding lock detents or grooves 1704 in the distal attachment flange portion 1700.

When employing an interchangeable shaft assembly that includes an end effector of the type described herein that is adapted to cut and fasten tissue, as well as other types of end effectors, it may be desirable to prevent inadvertent detachment of the interchangeable shaft assembly from the housing during actuation of the end effector. For example, in use the clinician may actuate the closure trigger 1032 to grasp and manipulate the target tissue into a desired position. Once the target tissue is positioned within the end effector 1300 in a desired orientation, the clinician may then fully actuate the closure trigger 1032 to close the anvil 2000 and clamp the target tissue in position for cutting and stapling. In that instance, the first drive system 1030 has been fully actuated. After the target tissue has been clamped in the end effector 1300, it may be desirable to prevent the inadvertent detachment of the shaft assembly 1200 from the housing 1012. One form of the latch system 1710 is configured to prevent such inadvertent detachment.

As can be most particularly seen in FIG. 4, the lock yoke 1712 includes at least one and preferably two lock hooks 1718 that are adapted to contact corresponding lock lug portions 1256 that are formed on the closure shuttle 1250. When the closure shuttle 1250 is in an unactuated position (i.e., the first drive system 1030 is unactuated and the anvil 2000 is open), the lock yoke 1712 may be pivoted in a distal direction to unlock the interchangeable shaft assembly 1200 from the housing 1012. When in that position, the lock hooks 1718 do not contact the lock lug portions 1256 on the closure shuttle 1250. However, when the closure shuttle 1250 is moved to an actuated position (i.e., the first drive system 1030 is actuated and the anvil 2000 is in the closed position), the lock yoke 1712 is prevented from being pivoted to an unlocked position. Stated another way, if the clinician were to attempt to pivot the lock yoke 1712 to an unlocked position or, for example, the lock yoke 1712 was inadvertently bumped or contacted in a manner that might otherwise cause it to pivot distally, the lock hooks 1718 on the lock yoke 1712 will contact the lock lug portions 1256 on the closure shuttle 1250 and prevent movement of the lock yoke 1712 to an unlocked position.

Attachment of the interchangeable shaft assembly 1200 to the handle 1014 will now be described. To commence the coupling process, the clinician may position the chassis 1240 of the interchangeable shaft assembly 1200 above or adjacent to the distal attachment flange portion 1700 of the frame 1020 such that the tapered attachment portions 1244 formed on the chassis 1240 are aligned with the dovetail slots 1702 in the frame 1020. The clinician may then move the shaft assembly 1200 along an installation axis that is perpendicular to the shaft axis SA to seat the attachment portions 1244 in “operable engagement” with the corresponding dovetail slots 1702. In doing so, the shaft attachment lug 1226 on the intermediate firing shaft portion 1222 will also be seated in the cradle 1126 in the longitudinally movable drive member 1120 and the portions of the pin 1037 on the second closure link 1038 will be seated in the corresponding hooks 1252 in the closure shuttle 1250. As used herein, the term “operable engagement” in the context of two components means that the two components are sufficiently engaged with each other so that upon application of an actuation motion thereto, the components may carry out their intended action, function and/or procedure.

At least five systems of the interchangeable shaft assembly 1200 can be operably coupled with at least five corresponding systems of the handle 1014. A first system can comprise a frame system which couples and/or aligns the frame 1020 or spine 1210 of the shaft assembly 1200 with the frame 1020 of the handle 1014. Another system can comprise a closure drive system 1030 which can operably connect the closure trigger 1032 of the handle 1014 and a closure tube of the shaft assembly 1200. As outlined above, the closure shuttle 1250 of the shaft assembly 1200 can be engaged with the pin 1037 on the second closure link 1038. Another system can comprise the firing drive system 1080 which can operably connect the firing trigger 1130 of the handle 1014 with the intermediate firing shaft portion 1222 of the shaft assembly 1200. As outlined above, the shaft attachment lug 1226 can be operably connected with the cradle 1126 of the longitudinal drive member 1120. Another system can comprise an electrical system which can signal to a controller in the handle 1014, such as microcontroller, for example, that a shaft assembly, such as shaft assembly 1200, for example, has been operably engaged with the handle 1014 and/or, two, conduct power and/or communication signals between the shaft assembly 1200 and the handle 1014. For instance, the shaft assembly 1200 can include an electrical connector 1810 that is operably mounted to the shaft circuit board 1610. The electrical connector 1810 is configured for mating engagement with a corresponding electrical connector 1800 on the circuit board 1100. Further details regarding the circuitry and control systems may be found in U.S. patent application Ser. No. 13/803,086, now U.S. Patent Application Publication No. 2014/0263541 entitled ARTICULATABLE SURGICAL INSTRUMENT COMPRISING AN ARTICULATION LOCK, and U.S. patent application Ser. No. 14/226,142, now U.S. Pat. No. 9,913,642 entitled SURGICAL INSTRUMENT COMPRISING A SENSOR SYSTEM, the entire disclosures of each which were previously incorporated by reference herein. The fifth system may consist of the latching system for releasably locking the shaft assembly 1200 to the handle 1014.

The anvil 2000 in the illustrated example includes an anvil body 2002 that terminates in an anvil mounting portion 2010. The anvil mounting portion 2010 is movably or pivotably supported on the elongate channel 1310 for selective pivotal travel relative thereto about a fixed anvil pivot axis PA that is transverse to the shaft axis SA. In the illustrated arrangement, a pivot member or anvil trunnion 2012 extends laterally out of each lateral side of the anvil mounting portion 2010 to be received in a corresponding trunnion cradle 1316 formed in the upstanding walls 1315 of the proximal end portion 1312 of the elongate channel 1310. The anvil trunnions 2012 are pivotally retained in their corresponding trunnion cradle 1316 by the channel cap or anvil retainer 1290. The channel cap or anvil retainer 1290 includes a pair of attachment lugs that are configured to be retainingly received within corresponding lug grooves or notches formed in the upstanding walls 1315 of the proximal end portion 1312 of the elongate channel 1310. See FIG. 5.

Still referring to FIG. 5, in at least one arrangement, the distal closure member or end effector closure tube 3050 employs two axially offset, proximal and distal positive jaw opening features 3060 and 3062. The positive jaw opening features 3060, 3062 are configured to interact with corresponding relieved areas and stepped portions formed on the anvil mounting portion 2010 as described in further detail in U.S. patent application Ser. No. 15/635,631, entitled SURGICAL INSTRUMENT WITH AXIALLY MOVABLE CLOSURE MEMBER, now U.S. Patent Application Publication No. 2019/0000464, the entire disclosure which has been herein incorporated by reference. Other jaw opening arrangements may be employed.

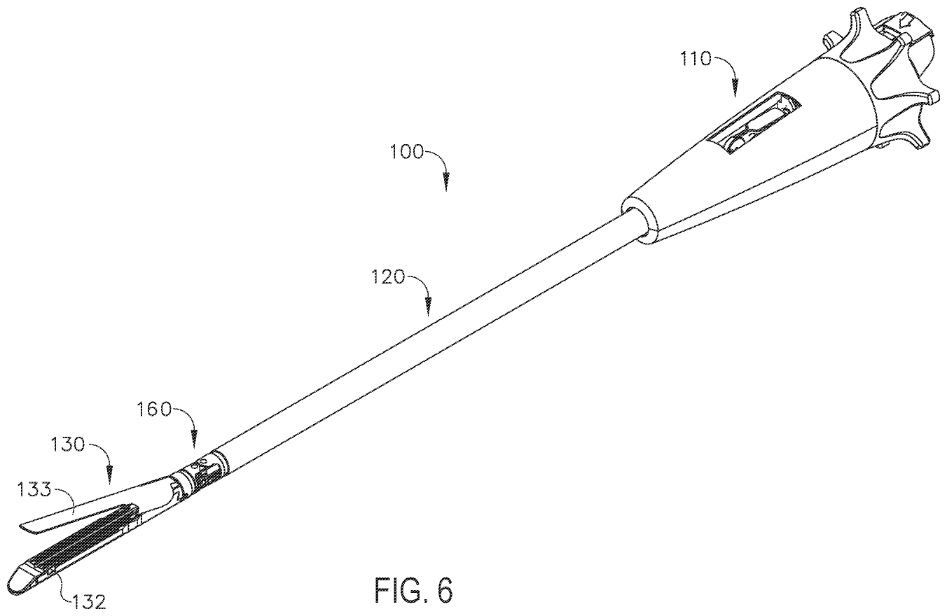

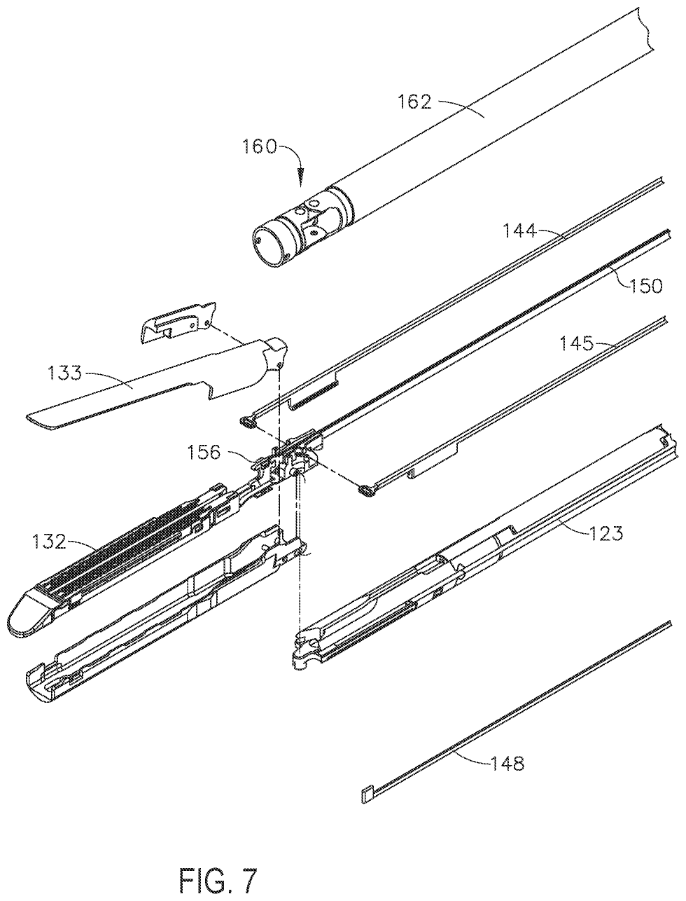

A shaft assembly 100 is illustrated in FIGS. 6 and 7. The shaft assembly 100 comprises an attachment portion 110, a shaft 120 extending distally from the attachment portion 110, and an end effector 130 attached to the shaft 120. The shaft assembly 100 is configured to clamp, staple, and cut tissue. The attachment portion 110 is configured to be attached to a handle of a surgical instrument and/or the arm of a surgical robot, for example.

Referring to FIG. 7, the shaft assembly 100 comprises cooperating articulation rods 144, 145 configured to articulate the end effector 130 relative to the shaft 120 about an articulation joint 160. The shaft assembly 100 further comprises an articulation lock bar 148, an outer shaft tube 162, and a spine portion 123.

Referring to FIG. 7, the shaft assembly 100 comprises a firing shaft 150 including a firing member 156 attached to a distal end of the firing shaft 150. The firing member 156 comprises upper camming flanges configured to engage an anvil jaw 133 and lower camming members configured to engage a cartridge jaw 132. The firing shaft 150 is configured to be advanced distally through a closure stroke to clamp the anvil jaw 133 relative to the cartridge jaw 132 with the camming members. Further advancement of the firing shaft 150 through a firing stroke is configured to advance the firing member 156 through the cartridge jaw 132 to deploy staples from the cartridge jaw 132 and cut tissue during the firing stroke. More details of the shaft assembly 100 can be found in U.S. patent application Ser. No. 15/385,887 entitled METHOD FOR ATTACHING A SHAFT ASSEMBLY TO A SURGICAL INSTRUMENT AND, ALTERNATIVELY, TO A SURGICAL ROBOT, which is incorporated by reference in its entirety.

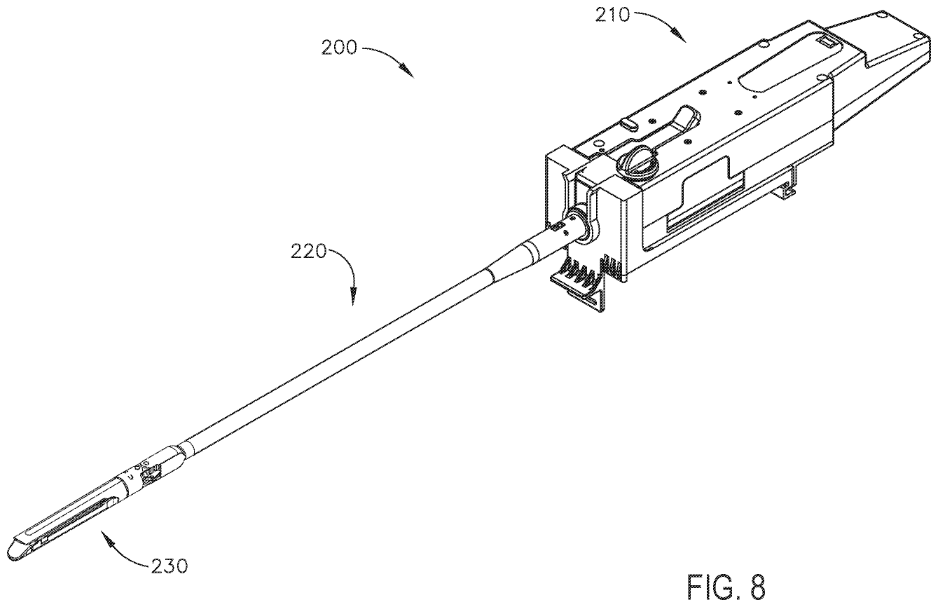

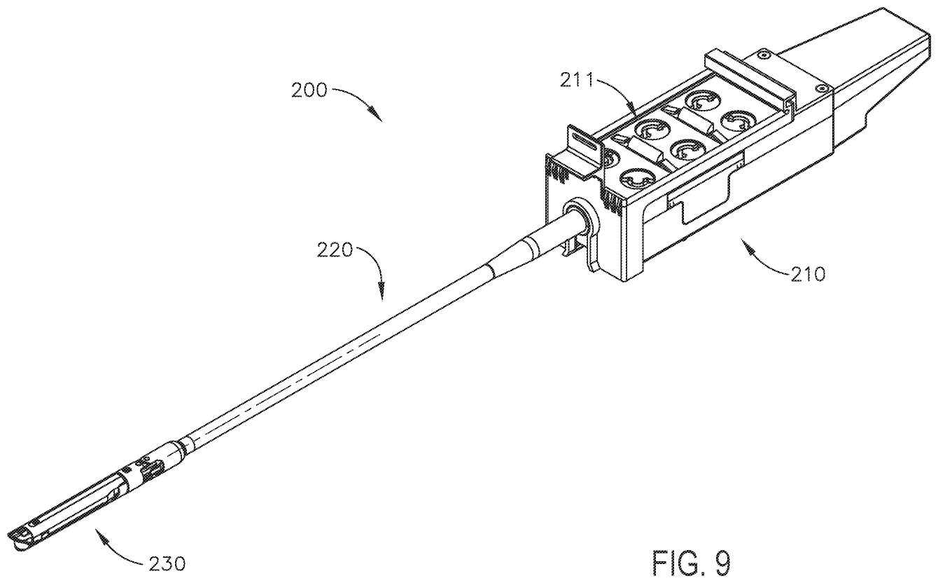

FIGS. 8 and 9 depict a surgical instrument assembly 200 configured to be used with a surgical robot. The surgical instrument assembly 200 is configured to staple and cut tissue, although the surgical instrument assembly 200 could be adapted to treat tissue in any suitable way, such as by applying heat energy, electrical energy, and/or vibrations to the tissue, for example. The surgical instrument assembly 200 comprises a proximal control interface 210 configured to be coupled to a robotic arm of a surgical robot and a shaft assembly 220 configured to be attached to the proximal control interface 210. The shaft assembly 220 comprises an end effector 230 configured to clamp, cut, and staple tissue. The proximal control interface 210 comprises a plurality of drive discs 211, each for actuating one or more functions of the surgical instrument assembly 200. Each drive disc 211 can be independently driven and/or cooperatively driven with one or more other drive discs 211 by one or more motors of the surgical robot and/or robotic arm of the surgical robot. More details about the surgical instrument assembly 200 can be found in U.S. patent application Ser. No. 15/847,297, entitled SURGICAL INSTRUMENTS WITH DUAL ARTICULATION DRIVERS, which is incorporated by reference in its entirety.

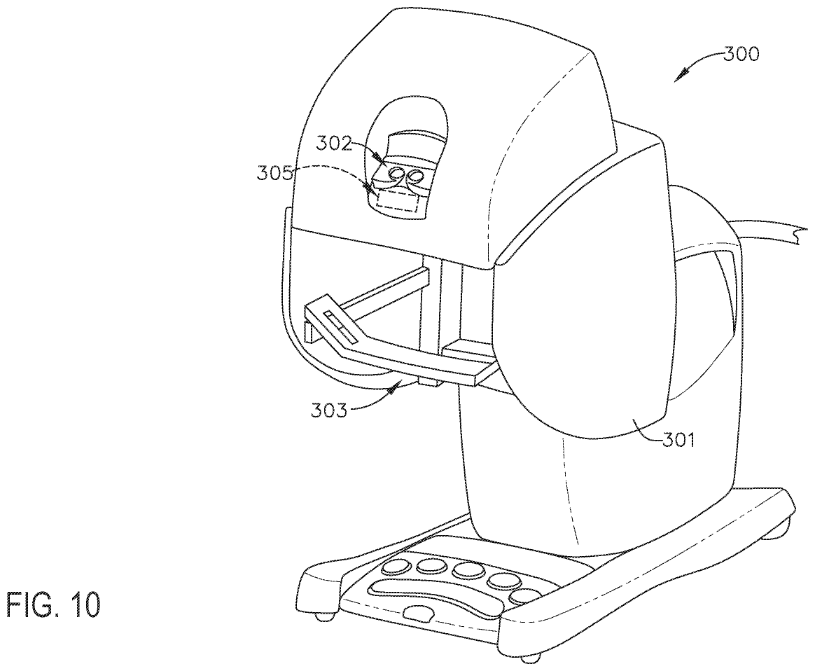

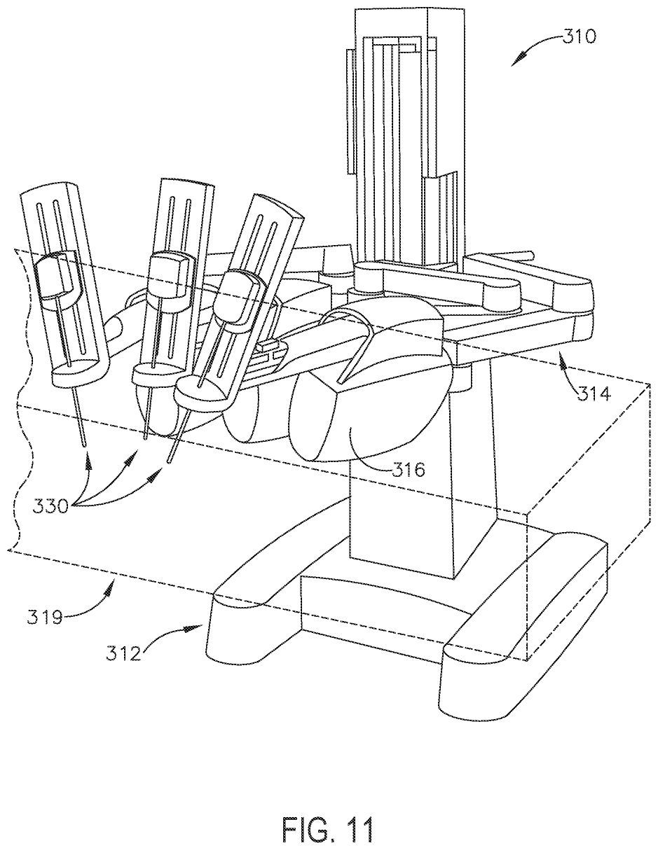

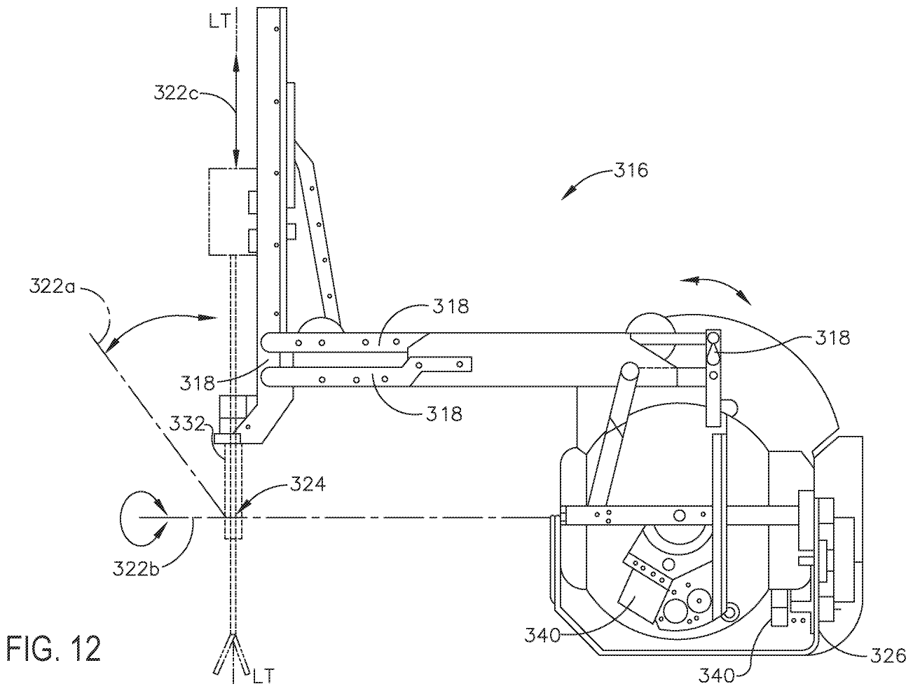

Various embodiments disclosed herein may be employed in connection with a robotic system 300 of the type depicted in FIGS. 10-12, for example. FIG. 10 depicts one version of a master controller 301 that may be used in connection with a robotic arm slave cart 310 of the type depicted in FIG. 11. Master controller 301 and robotic arm slave cart 310, as well as their respective components and control systems are collectively referred to herein as a robotic system 300. Examples of such systems and devices are disclosed in U.S. Pat. No. 7,524,320, entitled MECHANICAL ACTUATOR INTERFACE SYSTEM FOR ROBOTIC SURGICAL TOOLS, as well as U.S. Pat. No. 9,072,535, entitled SURGICAL STAPLING INSTRUMENTS WITH ROTATABLE STAPLE DEPLOYMENT ARRANGEMENTS, which are each hereby incorporated by reference herein in their respective entireties. Thus, various details of such devices will not be described in detail herein beyond that which may be necessary to understand various embodiments and forms of the present disclosure. As is known, the master controller 301 generally includes master controllers (generally represented as 303 in FIG. 10) which are grasped by the surgeon and manipulated in space while the surgeon views the procedure via a stereo display 302. The master controllers 301 generally comprise manual input devices which preferably move with multiple degrees of freedom, and which often further have an actuatable handle for actuating tools (for example, for closing grasping jaws, applying an electrical potential to an electrode, or the like).

As can be seen in FIG. 11, in one form, the robotic arm cart 310 may be configured to actuate one or more surgical tools, generally designated as 330. Various robotic surgery systems and methods employing master controller and robotic arm cart arrangements are disclosed in U.S. Pat. No. 6,132,368, entitled MULTI-COMPONENT TELEPRESENCE SYSTEM AND METHOD the entire disclosure of which is hereby incorporated by reference herein. In various forms, the robotic arm cart 310 includes a base 312 from which, in the illustrated embodiment, surgical tools may be supported. In various forms, the surgical tool(s) may be supported by a series of manually articulatable linkages, generally referred to as set-up joints 314, and a robotic manipulator 316. In various embodiments, the linkage and joint arrangement may facilitate rotation of a surgical tool around a point in space, as more fully described in issued U.S. Pat. No. 5,817,084, entitled REMOTE CENTER POSITIONING DEVICE WITH FLEXIBLE DRIVE, the entire disclosure of which is hereby incorporated by reference herein. The parallelogram arrangement constrains rotation to pivoting about an axis 322a, sometimes called the pitch axis. The links supporting the parallelogram linkage are pivotally mounted to set-up joints 314 (FIG. 11) so that the surgical tool further rotates about an axis 322b, sometimes called the yaw axis. The pitch and yaw axes 322a, 322b intersect at the remote center 324, which is aligned along an elongate shaft of a surgical tool. The surgical tool may have further degrees of driven freedom as supported by manipulator 316, including sliding motion of the surgical tool along the longitudinal axis “LT-LT”. As the surgical tool slides along the tool axis LT-LT relative to manipulator 316 (arrow 322c), remote center 324 remains fixed relative to base 326 of manipulator 316. Hence, the entire manipulator is generally moved to re-position remote center 324. Linkage 318 of manipulator 316 may be driven by a series of motors 340. These motors actively move linkage 318 in response to commands from a processor of a control system. The motors 340 may also be employed to manipulate the surgical tool. Alternative joint structures and set up arrangements are also contemplated. Examples of other joint and set up arrangements, for example, are disclosed in U.S. Pat. No. 5,878,193, entitled AUTOMATED ENDOSCOPE SYSTEM FOR OPTIMAL POSITIONING, the entire disclosure of which is hereby incorporated by reference herein. Additionally, while the data communication between a robotic component and the processor of the robotic surgical system is primarily described herein with reference to communication between the surgical tool and the master controller 301, it should be understood that similar communication may take place between circuitry of a manipulator, a set-up joint, an endoscope or other image capture device, or the like, and the processor of the robotic surgical system for component compatibility verification, component-type identification, component calibration (such as off-set or the like) communication, confirmation of coupling of the component to the robotic surgical system, or the like. In accordance with at least one aspect, various surgical instruments disclosed herein may be used in connection with other robotically-controlled or automated surgical systems and are not necessarily limited to use with the specific robotic system components shown in FIGS. 10-12 and described in the aforementioned references.

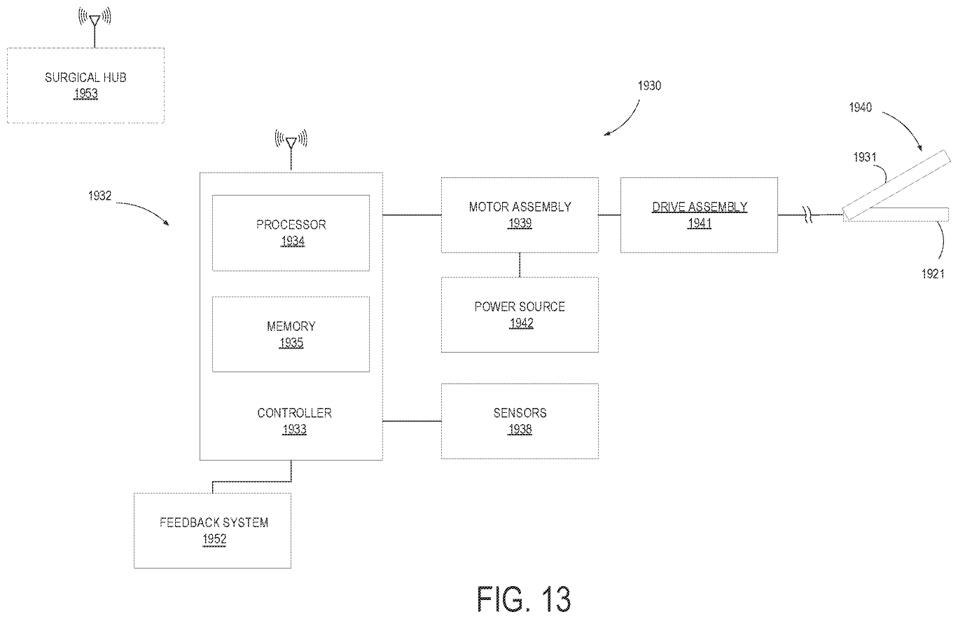

FIG. 13 illustrates a block diagram of a surgical system 1930 for use with one or more surgical instruments, tools, and/or robotic systems in accordance with one or more aspects of the present disclosure. The system 1930 includes a control circuit 1932. The control circuit 1932 includes a microcontroller 1933 comprising a processor 1934 and a storage medium such as, for example, a memory 1935.

A motor assembly 1939 includes one or more motors, driven by motor drivers. The motor assembly 1939 operably couples to a drive assembly 1941 to drive, or effect, one or more motions at an end effector 1940. The drive assembly 1941 may include any number of components suitable for transmitting motion to the end effector 1940 such as, for example, one or more linkages, bars, tubes, and/or cables, for example.

One or more of sensors 1938, for example, provide real-time feedback to the processor 1934 about one or more operational parameters monitored during a surgical procedure being performed by the surgical system 1930. The operational parameters can be associated with a user performing the surgical procedure, a tissue being treated, and/or one or more components of the surgical system 1930, for example. The sensor 1938 may comprise any suitable sensor, such as, for example, a magnetic sensor, such as a Hall effect sensor, a strain gauge, a pressure sensor, an inductive sensor, such as an eddy current sensor, a resistive sensor, a capacitive sensor, an optical sensor, and/or any other suitable sensor.

Further to the above, in various arrangements, the sensors 1938 may comprise any suitable sensor for detecting one or more conditions at the end effector 1940 including, without limitation, a tissue thickness sensor such as a Hall Effect Sensor or a reed switch sensor, an optical sensor, a magneto-inductive sensor, a force sensor, a pressure sensor, a piezo-resistive film sensor, an ultrasonic sensor, an eddy current sensor, an accelerometer, a pulse oximetry sensor, a temperature sensor, a sensor configured to detect an electrical characteristic of a tissue path (such as capacitance or resistance), or any combination thereof. As another example, and without limitation, the sensors 1938 may include one or more sensors located at, or about, an articulation joint extending proximally from the end effector 1940. Such sensors may include, for example, a potentiometer, a capacitive sensor (slide potentiometer), piezo-resistive film sensor, a pressure sensor, a pressure sensor, or any other suitable sensor type. In some arrangements, the sensor 1938 may comprise a plurality of sensors located in multiple locations in the end effector 1940.

In certain aspects, the system 1930 includes a feedback system 1952 which includes one or more devices for providing a sensory feedback to a user. Such devices may comprise, for example, visual feedback devices (e.g., an LCD display screen, a touch screen, LED indicators), audio feedback devices (e.g., a speaker, a buzzer) or tactile feedback devices (e.g., haptic actuators).

The microcontroller 1933 may be programmed to perform various functions such as precise control over the speed and position of the drive assembly 1941. In one aspect, the microcontroller 1933 may be any single-core or multicore processor such as those known under the trade name ARM Cortex by Texas Instruments. In one aspect, the main microcontroller 1933 may be an LM4F230H5QR ARM Cortex-M4F Processor Core, available from Texas Instruments, for example, comprising an on-chip memory of 256 KB single-cycle flash memory, or other non-volatile memory, up to 40 MHz, a prefetch buffer to improve performance above 40 MHz, a 32 KB single-cycle SRAM, and internal ROM loaded with StellarisWare® software, a 2 KB EEPROM, one or more PWM modules, one or more QEI analogs, and/or one or more 12-bit ADCs with 12 analog input channels, details of which are available for the product datasheet.

The microcontroller 1933 may be configured to compute a response in the software of the microcontroller 1933. The computed response is compared to a measured response of the actual system to obtain an “observed” response, which is used for actual feedback decisions. The observed response is a favorable, tuned value that balances the smooth, continuous nature of the simulated response with the measured response, which can detect outside influences on the system.

The motor assembly 1939 includes one or more electric motors and one or more motor drivers. The electric motors can be in the form of a brushed direct current (DC) motor with a gearbox and mechanical links to the drive assembly 1941. In one aspect, a motor driver may be an A3941 available from Allegro Microsystems, Inc.

In various forms, the motor assembly 1939 includes a brushed DC driving motor having a maximum rotational speed of approximately 25,000 RPM. In other arrangements, the motor assembly 1939 may include a brushless motor, a cordless motor, a synchronous motor, a stepper motor, or any other suitable electric motor. The motor driver may comprise an H-bridge driver comprising field-effect transistors (FETs), for example.

The motor assembly 1939 can be powered by a power source 1942. In certain aspects, the power source 1942 includes one or more batteries which may include a number of battery cells connected in series that can be used as the power source to power the motor assembly 1939. In certain circumstances, the battery cells of the power assembly may be replaceable and/or rechargeable. In at least one example, the battery cells can be lithium-ion batteries which can be couplable to and separable from the power assembly.

Further to the above, the end effector 1940 includes a first jaw 1921 and a second jaw 1931. At least one of the first jaw 1921 and the second jaw 1931 is rotatable relative to the other during a closure motion that transitions the end effector 1940 from an open configuration toward a closed configuration. The closure motion may cause the jaws 1921, 1931 to grasp tissue therebetween. In certain arrangements, sensors, such as, for example, a strain gauge or a micro-strain gauge, are configured to measure one or more parameters of the end effector 1940, such as, for example, the amplitude of the strain exerted on the one or both of the jaws 1921, 1931 during a closure motion, which can be indicative of the closure forces applied to the jaws 1921, 1931. The measured strain is converted to a digital signal and provided to the processor 1934, for example. Alternatively, additionally, sensors such as, for example, a load sensor, can measure a closure force and/or a firing force applied to the jaws 1921, 1931.

In various arrangements, a current sensor can be employed to measure the current drawn by a motor of the motor assembly 1939. The force required to advance the drive assembly 1941 can correspond to the current drawn by the motor, for example. The measured force is converted to a digital signal and provided to the processor 1934.

In one form, strain gauge sensors can be used to measure the force applied to the tissue by the end effector 1940, for example. A strain gauge can be coupled to the end effector 1940 to measure the force on the tissue being treated by the end effector 1940. In one aspect, the strain gauge sensors can measure the amplitude or magnitude of the strain exerted on a jaw of an end effector 1940 during a closure motion which can be indicative of the tissue compression. The measured strain is converted to a digital signal and provided to a processor 1934.

The measurements of the tissue compression, the tissue thickness, and/or the force required to close the end effector on the tissue, as respectively measured by the sensors 1938 can be used by the microcontroller 1933 to characterize the selected position of one or more components of the drive assembly 1941 and/or the corresponding value of the speed of one or more components of the drive assembly 1941. In one instance, a memory (e.g. memory 1935) may store a technique, an equation, and/or a lookup table which can be employed by the microcontroller 1933 in the assessment.

The system 1930 may comprise wired or wireless communication circuits to communicate with surgical hubs (e.g. surgical hub 1953), communication hubs, and/or robotic surgical hubs, for example. Additional details about suitable interactions between a system 1930 and the surgical hub 1953 are disclosed in U.S. patent application Ser. No. 16/209,423 entitled METHOD OF COMPRESSING TISSUE WITHIN A STAPLING DEVICE AND SIMULTANEOUSLY DISPLAYING THE LOCATION OF THE TISSUE WITHIN THE JAWS, now U.S. Patent Application Publication No. 2019/0200981, the entire disclosure of which is incorporated by reference in its entirety herein.

In various aspects, the control circuit 1932 can be configured to implement various processes described herein. In certain aspects, the control circuit 1932 may comprise a microcontroller comprising one or more processors (e.g., microprocessor, microcontroller) coupled to at least one memory circuit. The memory circuit stores machine-executable instructions that, when executed by the processor, cause the processor to execute machine instructions to implement various processes described herein. The processor may be any one of a number of single-core or multicore processors known in the art. The memory circuit may comprise volatile and non-volatile storage media. The processor may include an instruction processing unit and an arithmetic unit. The instruction processing unit may be configured to receive instructions from the memory circuit of this disclosure.

Alternatively, in certain instances, the control circuit 1932 can be in the form of a combinational logic circuit configured to implement various processes described herein. The combinational logic circuit may comprise a finite state machine comprising a combinational logic configured to receive data, process the data by the combinational logic, and provide an output.

Alternatively, in certain instances, the control circuit 1932 can be in the form of a sequential logic circuit. The sequential logic circuit can be configured to implement various processes described herein. The sequential logic circuit may comprise a finite state machine. The sequential logic circuit may comprise a combinational logic, at least one memory circuit, and a clock, for example. The at least one memory circuit can store a current state of the finite state machine. In certain instances, the sequential logic circuit may be synchronous or asynchronous. In other instances, the control circuit 1932 may comprise a combination of a processor (e.g., processor 1934) and a finite state machine to implement various processes herein. In other aspects, the finite state machine may comprise a combination of a combinational logic circuit (and the sequential logic circuit, for example.

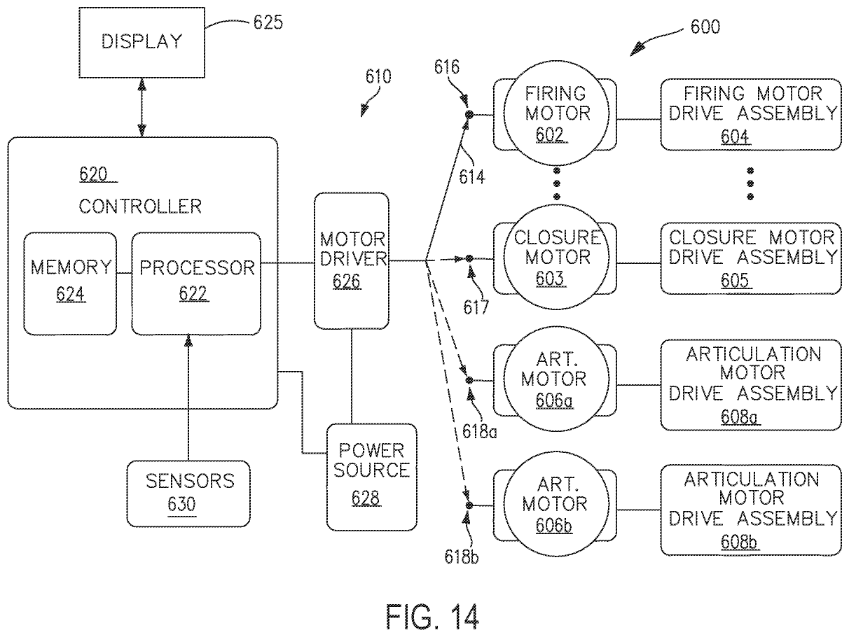

FIG. 14 illustrates a block diagram of a surgical system 600 for use with one or more surgical instruments, tools, and/or robotic systems in accordance with one or more aspects of the present disclosure. The surgical system 600 is similar in many respects to the surgical system 1930, which are not repeated herein at the same of detail for brevity. For example, like the surgical system 1930, the surgical system 600 includes a control circuit comprising a microcontroller 620 comprising a processor 622 and a memory 624, sensors 630, and a power source 628, which are similar, respectively, to the microcontroller 1933, the processor 1934, the memory 1935, and the power source 1942. Additionally, the surgical system 600 includes a plurality of motors and corresponding driving assemblies that can be activated to perform various functions.

In certain instances, a first motor can be activated to perform a first function, a second motor can be activated to perform a second function, a third motor can be activated to perform a third function, a fourth motor can be activated to perform a fourth function, and so on. In certain instances, the plurality of motors can be individually activated to cause firing, closure, and/or articulation motions in an end effector 1940, for example. The firing, closure, and/or articulation motions can be transmitted to the end effector 1940 through a shaft assembly, for example.

In certain instances, the system 600 may include a firing motor 602. The firing motor 602 may be operably coupled to a firing motor drive assembly 604 which can be configured to transmit firing motions, generated by the motor 602 to the end effector, in particular to displace the I-beam element. In certain instances, the firing motions generated by the motor 602 may cause the staples to be deployed from a staple cartridge into tissue captured by the end effector 1940 and/or the cutting edge of the I-beam element to be advanced to cut the captured tissue, for example. The I-beam element may be retracted by reversing the direction of the motor 602.

In certain instances, the system 600 may include a closure motor 603. The closure motor 603 may be operably coupled to a closure motor drive assembly 605 which can be configured to transmit closure motions, generated by the motor 603 to the end effector 1940, in particular to displace a closure tube to close an anvil and compress tissue between the anvil and the staple cartridge. The closure motions may cause the end effector 1940 to transition from an open configuration to an approximated configuration to grasp tissue, for example. The end effector 1940 may be transitioned to an open position by reversing the direction of the motor 603.

In certain instances, the system 600 may include one or more articulation motors 606a, 606b, for example. The motors 606a, 606b may be operably coupled to respective articulation motor drive assemblies 608a, 608b, which can be configured to transmit articulation motions generated by the motors 606a, 606b to the end effector. In certain instances, the articulation motions may cause the end effector to articulate relative to a shaft, for example.

As described above, the system 600 may include a plurality of motors which may be configured to perform various independent functions. In certain instances, the plurality of motors of the surgical instrument or tool can be individually or separately activated to perform one or more functions while the other motors remain inactive. For example, the articulation motors 606a, 606b can be activated to cause the end effector to be articulated while the firing motor 602 remains inactive. Alternatively, the firing motor 602 can be activated to fire the plurality of staples, and/or to advance the cutting edge, while the articulation motor 606 remains inactive. Furthermore, the closure motor 603 may be activated simultaneously with the firing motor 602 to cause the closure tube and the I-beam element to advance distally as described in more detail hereinbelow.

In certain instances, the system 600 may include a common control module 610 which can be employed with a plurality of motors of the surgical instrument or tool. In certain instances, the common control module 610 may accommodate one of the plurality of motors at a time. For example, the common control module 610 can be couplable to and separable from the plurality of motors of the robotic surgical instrument individually. In certain instances, a plurality of the motors of the surgical instrument or tool may share one or more common control modules such as the common control module 610. In certain instances, a plurality of motors of the surgical instrument or tool can be individually and selectively engaged with the common control module 610. In certain instances, the common control module 610 can be selectively switched from interfacing with one of a plurality of motors of the surgical instrument or tool to interfacing with another one of the plurality of motors of the surgical instrument or tool.

In at least one example, the common control module 610 can be selectively switched between operable engagement with the articulation motors 606a, 606b and operable engagement with either the firing motor 602 or the closure motor 603. In at least one example, as illustrated in FIG. 14, a switch 614 can be moved or transitioned between a plurality of positions and/or states. In a first position 616, the switch 614 may electrically couple the common control module 610 to the firing motor 602; in a second position 617, the switch 614 may electrically couple the common control module 610 to the closure motor 603; in a third position 618a, the switch 614 may electrically couple the common control module 610 to the first articulation motor 606a; and in a fourth position 618b, the switch 614 may electrically couple the common control module 610 to the second articulation motor 606b, for example. In certain instances, separate common control modules 610 can be electrically coupled to the firing motor 602, the closure motor 603, and the articulations motor 606a, 606b at the same time. In certain instances, the switch 614 may be a mechanical switch, an electromechanical switch, a solid-state switch, or any suitable switching mechanism.

Each of the motors 602, 603, 606a, 606b may comprise a torque sensor to measure the output torque on the shaft of the motor. The force on an end effector may be sensed in any conventional manner, such as by force sensors on the outer sides of the jaws or by a torque sensor for the motor actuating the jaws.

In various instances, as illustrated in FIG. 14, the common control module 610 may comprise a motor driver 626 which may comprise one or more H-Bridge FETs. The motor driver 626 may modulate the power transmitted from a power source 628 to a motor coupled to the common control module 610 based on input from a microcontroller 620 (the “controller”), for example. In certain instances, the microcontroller 620 can be employed to determine the current drawn by the motor, for example, while the motor is coupled to the common control module 610, as described above.

In various instances, the processor 622 may control the motor driver 626 to control the position, direction of rotation, and/or velocity of a motor that is coupled to the common control module 610. In certain instances, the processor 622 can signal the motor driver 626 to stop and/or disable a motor that is coupled to the common control module 610.

In certain instances, the memory 624 may include program instructions for controlling each of the motors of the surgical instrument 600 that are couplable to the common control module 610. For example, the memory 624 may include program instructions for controlling the firing motor 602, the closure motor 603, and the articulation motors 606a, 606b. Such program instructions may cause the processor 622 to control the firing, closure, and articulation functions in accordance with inputs from algorithms or control programs of the surgical instrument or tool.

In certain instances, one or more mechanisms and/or sensors such as, for example, sensors 630 can be employed to alert the processor 622 to the program instructions that should be used in a particular setting. For example, the sensors 630 may alert the processor 622 to use the program instructions associated with firing, closing, and articulating the end effector. In certain instances, the sensors 630 may comprise position sensors which can be employed to sense the position of the switch 614, for example. Accordingly, the processor 622 may use the program instructions associated with firing the I-beam of the end effector upon detecting, through the sensors 630 for example, that the switch 614 is in the first position 616; the processor 622 may use the program instructions associated with closing the anvil upon detecting, through the sensors 630 for example, that the switch 614 is in the second position 617; and the processor 622 may use the program instructions associated with articulating the end effector upon detecting, through the sensors 630 for example, that the switch 614 is in the third or fourth position 618a, 618b.

As discussed above, surgical devices can record a plurality of surgical data during a surgical procedure. As surgical devices have become “smarter” there has been an increase in the size of data recorded and analyzed, in some instances, beyond the capabilities of the surgical devices within a suitable time frame. In such instances, distributed processing is sought as one solution for processing data beyond the capabilities of a surgical device that generated, or received, the surgical data. Thus the processing can be shared allowing larger datasets to be processed in real-time. In addition to processing larger datasets, the decentralized processing can decrease part cost and increase modular functionality between devices.

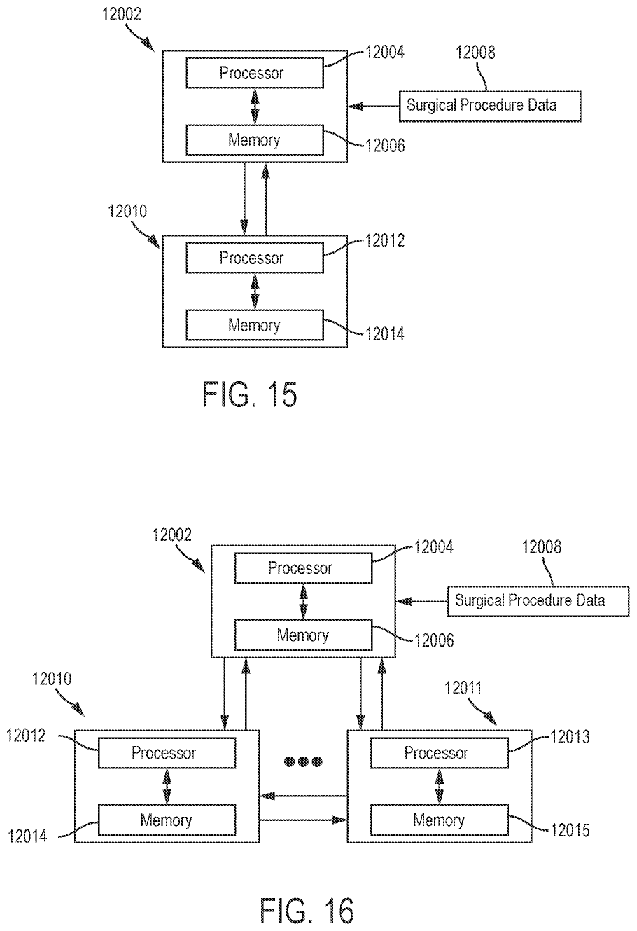

FIG. 15 illustrates a diagram of a distributed processing system 12000 that includes at least two control circuits 12002, 12010. These control circuits 12002, 12010 can be associated with a surgical device, surgical hub, or remote processing device. For example, one of the control circuits 12002, 12010 could be control circuit 1932 (FIG. 13), surgical hub 1953 (FIG. 13), or control circuit 620 (FIG. 14). The control circuit 12002 has a processor 12004 coupled to a memory 12006 and the control circuit 12010 has a processor 12012 coupled to a memory 12014. In one aspect, the processor 12004 can act as a master processor and processor 12012 can act as a follower, or slave, processor.

As illustrated, the control circuit 12002 receives surgical procedure data 12008. The surgical procedure data 12008 could be any data received from surgical devices and/or users related to the surgical procedure. In one aspect, the surgical procedure data 12008 includes data representing sensor readings from one or more sensors associated with one or more surgical devices. In one aspect, the surgical procedure data 12008 includes imaging data from one or more imaging devices. In certain instances, the imaging data can represent single or multiple frames of a surgical site. The imaging data can represent single or multiple frames taken before and/or during the surgical procedure.

In the illustrated example, the processor 12004 elects a distributed processing of the surgical procedure data 12008 due to the large size of the surgical procedure data 12008 and/or due to the limited available processing time. As such, the control circuit 12002 sends a subset of the surgical procedure data to the control circuit 12010. The processor 12012 performs an analysis on that subset of data and sends the result to the control circuit 12002. In one aspect, a specific task is requested of the follower processor 12012 by the master processor 12004. While the processor 12012 performs an analysis, the processor 12004 performs a different analysis on a different subset of the surgical procedure data. Thus, the processing is distributed between the two control circuits 12002, 12010.

The distributed processing can be extended to any number of control circuits. FIG. 16 illustrates the same distributed processing approach shown in FIG. 15 but applied to multiple follower, or slave, processors. Similar to FIG. 15, the processor 12004 can act as a master processor and the processor 12012 can act as a follower processor; however, now there is an additional follower processor 12013. Each of these control circuits 12002, 12010, 12011 could be from a surgical device, surgical hub, and/or remote processing device. For example, one of the control circuits 12002, 12010, 12011 could be control circuit 1932 (FIG. 13), surgical hub 1953 (FIG. 13), or control circuit 620 (FIG. 14). The control circuit 12011 functions similar to control circuit 12010. The control circuit 12011 has the processor 12013 coupled to a memory 12015.

As illustrated, the control circuit 12002 sends a first subset of the surgical procedure data to the control circuit 12010 and a second subset of the surgical procedure data to the control circuit 12011. In one aspect, one task is requested of the follower processor 12012 by the master processor 12004 and a different task is requested from processor 12013 by the master processor 12004. The processor 12012 performs an analysis on the first subset of data and sends the result to the control circuit 12002. The processor 12013 performs a different analysis on the second subset of data and sends the result to the control circuit 12002. While the processors 12012, 12013 perform analyses, the processor 12004 can perform a third analysis on a third subset of the surgical procedure data. Thus, the processing is distributed between the three control circuits. However, this approach can be expanded to include any number of follower processors.

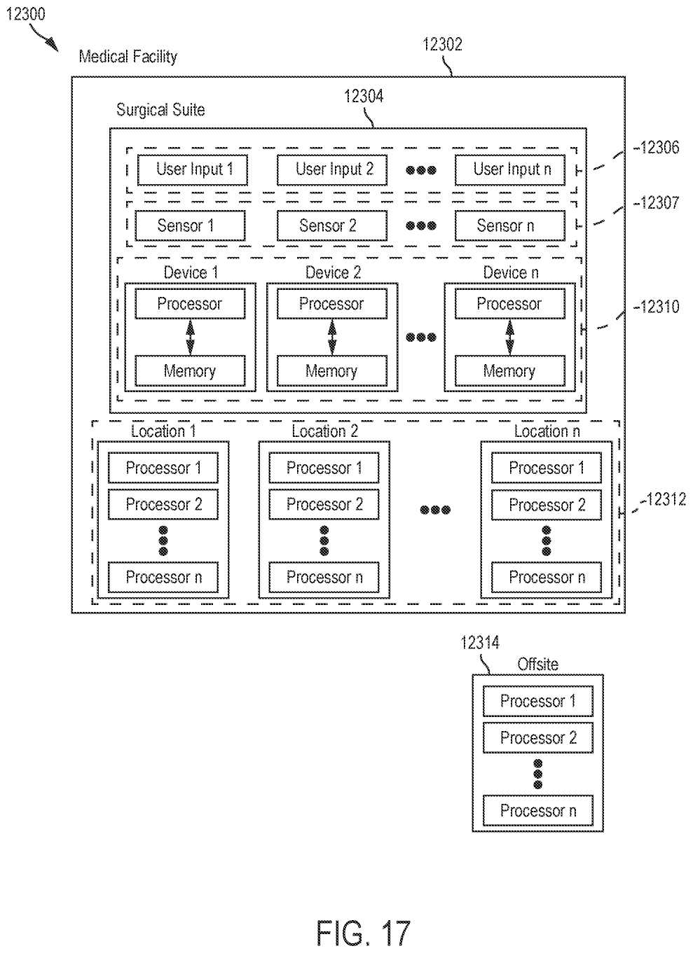

FIG. 17 illustrates a diagram 12300 depicting some of the potential control circuits that a master processor has access to for distributed processing in a surgical suite 12304 of a medical facility 12302. The surgical procedure data 12008 can come from any number of sensors 12307 and/or user inputs 12306. In at least one aspect, the sensors 12307 are located on different devices in the surgical suite 12304. In at least one aspect, the user inputs 12306 come from a variety of devices in the surgical suite that a person can access. The data from the sensors 12307 and the data from the user inputs 12306 are received by the control circuit 12002 and master processor 12004 and used as inputs to determine control adjustments to a surgical device.

The master processor 12004 (FIG. 15) can perform distributed processing with any number of devices 12310 in the surgical suite 12304, where each of these devices has a processor coupled to a memory. In one aspect, these devices 12310 are surgical instruments in the surgical suite 12304 that are not overly burdened and have processor capacity remaining. In some aspects, a medical facility 12302 has dedicated processor farms 12312 at different locations. For example, the dedicated processor farm location 1 can be dedicated to data processing for operating rooms of the hospital, while location 2 can be dedicated to data processing for intensive care units of the hospital, and etc. The master processor 12004 can perform distributed processing with one of these processor farms 12312 located within the medical facility 12302. The control circuit 12002 and the master processor 12004 can also perform distributed processing with an offsite processor farm 12314 that is located outside of the medical facility 12302.

Various methods and systems associated with performing distributed processing are described in U.S. Pat. No. 11,419,630, titled SURGICAL SYSTEM DISTRIBUTED PROCESSING, issued Aug. 23, 2022, which is incorporated by reference herein in its entirety.

One of the benefits of performing distributed processing is processing decentralization to decrease part cost and increase modular functionality. For example, allowing the processing to be spread across multiple processors allows the individual processors to be smaller and thus cheaper. One approach to decentralizing processing is to offload software, or processes, to appropriate processors based on utilization needs. For example, a processor that is not being used at a specific time in a surgical procedure could be processing data for a device that is being used. A “dummy” device can also be created that can be used for distributed processing in a surgical suite. This device is added into the surgical suite and can have the sole purpose of being used for distributed processing. A master processor relationship is created, where a processor that is not fully utilized connects to a master processor and performs distributed processing as described in FIGS. 15 and 16.

For some processes it can be beneficial to have a closed loop system. For example, a process that requires a quick response time could benefit from a dedicated high frequency micro-controller that could run in a closed loop system control. This could allow any response from the micro-controller to be as fast as possible. In some aspects, the micro-controller could be separate from any distributed processing.

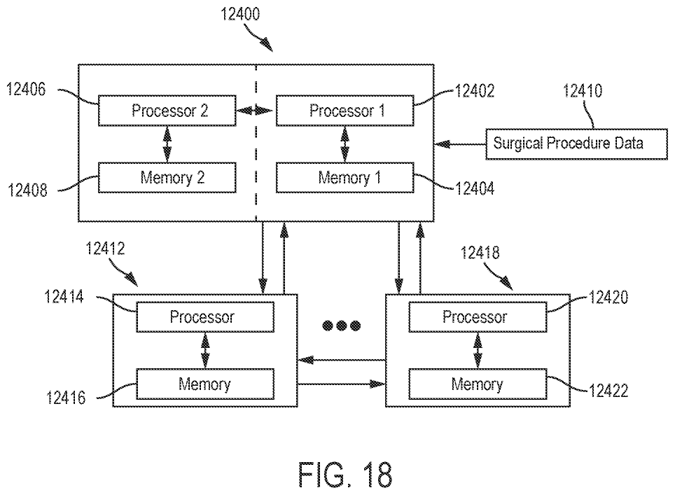

An example process that requires a quick response time is safety monitoring, which needs to occur constantly. Having a dedicated safety monitoring system can be beneficial to the safety of the patient and the device. The tasks that are associated with safety activities could be isolated from the potential data distribution and kept locally on the device. In one aspect, the processing of safety tasks is executed via a dedicated thread on a device with no parallel processing. FIG. 18 illustrates a device, e.g. surgical instrument 1010 (FIG. 1), surgical instrument assembly 200 (FIG. 8), control circuit 1992 (FIG. 13), surgical hub 1953 (FIG. 13), or control circuit 620 (FIG. 14), that participates in distributed processing for some tasks but also has a dedicated thread outside of the distributed processing. The distributed processing includes control circuits 12400, 12412, 12418. Each of these control circuits 12400, 12412, 12418 could be from a surgical device, surgical hub, or remote processing device. For example, one of the control circuits 12400, 12412, 12418 could be control circuit 1932 (FIG. 13), surgical hub 1953 (FIG. 13), or control circuit 620 (FIG. 14).

The control circuit 12400 has at least two processors 12402, 12406 coupled to two memories 12404, 12408, respectively. The control circuits 12412, 12418 have processors 12414, 12420 coupled to memory units 12416, 12422, respectively. As described in connection to FIG. 16, the processor 12402 acts as a master processor and processors 12414, 12420 act as follower processors. The control circuit 12400 receives surgical procedure data 12410, and performs distributed processing with control circuits 12412, 12418. The master processor 12402 isolates tasks associated with safety activities, and provides these tasks to processor 12406 to be completed locally on the device. The master processor 12402 distributes the non-safety related tasks to the distributed processing.

In various aspects, a process, which can be executed by the processor 12402, includes receiving surgical data, determining whether the received surgical data is associated with a safety task. In one aspect, the process includes categorizing the surgical data as safety data or non-safety, or functional, data. The process further includes transmitting the safety data to a dedicated processor (e.g. 12406), and transmitting the non-safety data to another non-dedicated, or general, processor (e.g. 12414, 12420) for distributed processing. This process allows distributed processing between multiple control circuits, while keeping the safety monitoring locally with processor 12406. This approach can be expanded to include any number of follower processors.

In various aspects, the surgical data may include a safety tag or label that permits identification of the surgical data as safety data. Additionally, or alternatively, determining that a received surgical data is safety data can be achieved based on the source of the surgical data. For example, surgical data from particular sensors can be automatically considered safety data.

Referring still to FIG. 18, if the control circuit 12400 did not have processor 12406 and memory 12408, the safety monitoring could still be kept local to the device and not part of the distributed processing. For example, while the processors 12414, 12420 perform analyses, the processor 12402 performs the safety monitoring tasks. This process would allow the processing to be distributed between the three control circuits, while keeping the safety monitoring local to the device, for example.

Another benefit of distributed processing is that there could be a dedicated device, or control circuit, tasked with data storage. The distributed processing can function similar to the distributed processing described in connection to FIG. 16. However, in this instance, the control circuit 12010 is utilized as a dedicated storage device tasked with storing the surgical procedure data 12008 and any other data sent to the control circuit 12010. In some aspects, the control circuit 12010 could receive sensor data from sensors (e.g. sensors 12307, FIG. 17), user inputs (e.g. user inputs 12306, FIG. 17), and devices (e.g. devices 12310, FIG. 17) in the surgical suite during a surgery. In some instances, the master control circuit 12002 and follower control circuit 12011 route surgical data to the control circuit 20210 for storage in the memory 12014, for example.

In some aspects, the control circuit 12010 stores all the data that is being generated during a surgical procedure. Having a dedicated storage device in the surgical suite allows other devices in the surgical suite to not spend processor capacity on data storage and/or categorization, and instead focus on data processing. In some aspects, the control circuit 12010 categorizes the received data and/or assigns labels to the received data. In at least one aspect, the control circuit 12010 stores the data in a database. The database allows the user, the control circuit 12002, and/or the control circuit 12011 to query desired information and analyze it along with other data in the database anytime during or after the surgery. In some aspects, the control circuit 12010 anonymizes the database and transmits a summary to a remote server or surgical hub, e.g. surgical hub 1953 (FIG. 13).

Another benefit of performing distributed processing is increasing modular functionality. Different processors can be assigned different processing tasks in a decentralized processing environment. As illustrated in FIG. 17, a surgical suite 12304 includes devices 12310 and sensors 12307 that are used during a surgical procedure. Decentralized processing allows data from these devices and sensors to be processed faster and to be communicated where needed automatically, which can improve overall surgical outcomes. For example, a surgical stapling and cutting device, e.g. surgical instrument 1010 or surgical instrument assembly 200, which is used to perform a tissue treatment motion that seals and cuts tissue at a surgical site in a patient, can benefit from surgical data indicative of the patient blood pressure. If blood pressure of the patient can be abnormally high, it is desirable to delay sealing and cutting a tissue including a blood vessel, for example, until the blood pressure stabilizes. In one aspect, a control circuit, e.g. control circuit 1932 (FIG. 13), of the surgical device overrides a user input to begin a firing stroke for sealing and cutting the tissue based on the detection of the blood vessel in the tissue and the detection of a blood pressure higher than a predetermined value.

Further to the above, overriding the user input requires simultaneous data processing of surgical data from multiple sources such as, for example, data from a blood pressure sensor, that can be in a separate device or on the surgical device, data from one or more imaging devices, data from a user input, and/or data from the surgical device indicative of a clamped state of an end effector grasping the tissue to be treated. In one aspect, all the data are received by the control circuit of the surgical device, and all the data processing is performed by a single processor of the surgical device. In other aspects, however, a decentralized approach is taken as discussed in connection with FIGS. 15 and 16, wherein the blood pressure sensor data and/or the imaging data are analyzed at separate processors, independent from the processor of the surgical device. In such aspects, the control circuit of the surgical device receives from a first independent processor a communication indicating that the blood pressure is equal to or higher than a predetermined threshold, for example, and received from a second independent processor a communication indicating the detection of a blood vessel in the tissue being grasped by an end effector of the surgical device. The separate processing of the blood pressure sensor data and the imaging data, external to the control circuit of the surgical device, facilitates a timely override of the user input to begin the firing stroke, or at least a timely pause to permit the user to make an informed decision to continue the firing stroke, or wait until the blood pressure stabilizes.

Additionally, or alternatively, the surgical device control circuit adjusts the tissue treatment motion based on the blood pressure. For example, the control circuit can decrease the speed of the tissue treatment motion or decrease the force applied to the tissue during the tissue treatment motion based on the blood pressure.

Further to the above, a similar override of the firing stroke can be performed by the control circuit of the surgical device based on the detection of a solid object such as, for example, previously fired staples or clips in a tissue grasped by an end effector of the surgical device. Imaging data can be analyzed by a separate processor that identifies the presence of a solid object in the tissue being grasped by the end effector. Additionally, or alternatively, sensor data indicative of the presence of the solid object can also be processed by the same, or a different, separate processor.

Further to the above, the control circuit of the surgical device overrides a user input to begin the firing stroke based on a communication from the separate processor(s) indicating the presence of the solid object. Additionally, the control circuit may alert the user to the presence of the solid object and may request an input as to whether the user wishes to continue the firing stroke. In certain instances, the communication from the separate processor(s) can include an image showing the solid object, or information regarding the position of the solid object with respect to the tissue being grasped by the end effector. The control circuit may present, through a user interface, the image and/or the information to the user along with a request for a decision as to whether or not to perform the firing stroke.

In various aspects, similar surgical situations can be benefit from a decentralized approach of sensor and/or imaging data processing by one or more control circuits communicably coupled to the control circuit of a surgical device. For example, a surgical hub, e.g. surgical hub 1953 (FIG. 13), control circuit could monitor sensor information and adjust a surgical device, e.g. surgical instrument 1010 (FIG. 1), surgical instrument assembly 200 (FIG. 8), control circuit 1992 (FIG. 13), or control circuit 620 (FIG. 14), based on the sensor information. In at least one aspect, the surgical hub receives data from multiple sources including surgical devices (e.g. devices 12310), sensors (e.g. sensors 12307), and surgeons (e.g. user inputs 12306). The surgical hub makes adjustments to the surgical device based on the received data. In one aspect, the surgical hub stops and/or delays the tissue treatment motion of the surgical device until a predefined threshold is reached. Additionally, or alternatively, the surgical hub adjusts the tissue treatment motion of the surgical device based on the data received.

The decentralized processing approach allows additional sensors to become part of a surgical device, e.g. surgical instrument 1010 (FIG. 1), surgical instrument assembly 200 (FIG. 8), or surgical hub 1953 (FIG. 13), without having to update the hardware of the device. This is due to the surgical device being communicably coupled to different devices and having the capability to have more data analyzed through distributed processing. In at least one aspect, the surgical device control circuit is communicably coupled to a new sensor that is added to the surgical device or surgical procedure. The new sensor provides data to the control circuit, e.g. control circuit 1992 (FIG. 13), surgical hub 1953 (FIG. 13), or control circuit 620 (FIG. 14), of the surgical device that can improve surgical outcomes. In one aspect, the new sensor provides more accurate data than a sensor already connected to the surgical device. In such aspect, distributed processing, as described in connection to FIGS. 15 and 16, can be employed to analyze the new data and prioritize the improved data results over the old data result from the less reliable sensor. This process improves the performance of a surgical device without upgrading or replacing the surgical device itself. If multiple sensors, or devices, are added to the device system, then some of these additions can be used to ensure that there is a backup component if a primary component fails. In some aspects, a component from a first device system is removed from the first device system and added to a second device system due to a failure of a component in the second device system.

Further to the above, the decentralized processing approach permits a control circuit of a surgical device to benefit from contextual perioperative data about a surgical procedure that impacts the algorithms within a surgical device, e.g. surgical instrument 1010 (FIG. 1) or surgical instrument assembly 200 (FIG. 8), without overloading a processor of the control circuit of the surgical device with additional processing tasks. For example, a surgeon supplies information to a control circuit, e.g. control circuit 1992 (FIG. 13), surgical hub 1953 (FIG. 13), or control circuit 620 (FIG. 14), through a user interface. In some aspects, the user interface could be a surgical hub, e.g. surgical hub 1953 (FIG. 13). The data from the surgeon is received by the control circuit and used in distributed processing, as described in connection to FIGS. 15 and 16, with data from other sources, for example from sensors and devices in the surgical suite. The control circuit receives results on different analyses performed on the data received by the control circuit. The data from the surgeon and the results allow the control circuit to make control adjustments to the surgical device during the surgical procedure. For example, the surgeon inputs information about the staple cartridge that is being used during the surgical procedure. The information is processed in a separate processor, and an outcome of the data processing is communicated to the control circuit of the surgical device. The control circuit adjusts various operational parameters of the surgical device based on processed data associated with the staple cartridge. The control circuit then uses the determined parameters to perform the tissue treatment motion that accounts for all the information received by the control circuit including the staple cartridge information supplied by the surgeon.

The decentralized processing approach allows for some processing to be performed by compiler/processor farms. Referring back to FIG. 17, the dedicated processor farms 12312 are located in the medical facility 12302 and can be decentralized to high activity zones in the medical facility 12302 such as operating rooms, intensive care units, emergency rooms, and etc. By having a dedicated processor farm for each of these departments in the hospital, the processor farms become strategically positioned to better handle the data within the hospital. It is beneficial to allow surgical devices, e.g. surgical instrument 1010 (FIG. 1), surgical instrument assembly 200 (FIG. 8), control circuit 1992 (FIG. 13), surgical hub 1953 (FIG. 13), or control circuit 620 (FIG. 14), in high activity zones in a hospital to have access to multiple dedicated processor farms, to avoid bandwidth issues. For example, there is a limit to the amount of data that can be sent at once to a processor farm. Thus, having more than one processor farm allows different devices in the medical facility to have access to a processor farm even if there is not bandwidth to communicate with one of them.

Additionally, having a dedicated processor farm for a specific zone, such as operating rooms, allows the processor farm to prioritize processing data for that specific zone. For example, the processor farm could prioritize processing for a control circuit located in the specific zone over another control circuit located outside of the zone. In at least one example, a processor farm associated with a first zone, e.g. operating rooms, in a medical facility receives a first processing request of a first data from a first control circuit located in the first zone, and a second processing request of a second data from a second control circuit located outside the first zone or in a second zone such as emergency rooms. In one instance, the processor farm priories the first request over the second request based on location information. The processing requests can be tagged or accompanied by location information of their origin control circuits.

In one instance, the processor farm further considers the priority level of the first and second requests. The priority level can be a risk priority level. For example, a device that is in an operation where someone is undergoing emergency life-saving surgery has a higher priority access to the processor farm than a device being used in a routine low risk surgery. This approach allows the processor farm to benefit the people that are the most at risk. However, other priority schematics can be envisioned to allow devices access to the processor farms, when needed.

In some aspects, referring again to FIG. 15, the follower device can be remote from the master device. In one aspect, the master device is a surgical hub, e.g. surgical hub 1953 (FIG. 13), and the follower device is a remote processing device such as, for example, a processor farm. The follower device can also be a surgical device, e.g. surgical instrument 1010 (FIG. 1) or surgical instrument assembly 200 (FIG. 8), with a control circuit, e.g. control circuit 1992 (FIG. 13) or control circuit 620 (FIG. 14). In another aspect, the master device is a surgical device, e.g. surgical instrument 1010 (FIG. 1) or surgical instrument assembly 200 (FIG. 8), with a control circuit, e.g. control circuit 1992 (FIG. 13) or control circuit 620 (FIG. 14), and the follower device is a surgical hub, e.g. surgical hub 1953 (FIG. 13) and/or a processor farm. During distributed processing, as described in connection to FIGS. 15 and 16, follower devices send results, or post processed data, to the master device for use in control of a surgical instrument subsystem, for example, where the master device combines the result, or post-processed data, from a follower device with other results and post processed data from another follower device, and/or results and post processed data from the master device, to execute a control algorithm for controlling a surgical device, for example. For this process to function properly, a master processor and a follower processor need to synchronize so that any results sent between them are accurately used. The synchronization insures the prevention of out of sequence combinations of results or processes feeding information to a control circuit of a surgical device.

A control system can couple to a remote processing device to execute distributed processing. In one aspect, the control system is a surgical hub, e.g. surgical hub 1953 (FIG. 13). In another aspect, the control system is a control circuit of a surgical device, e.g. control circuit 1932 (FIG. 13) or control circuit 620 (FIG. 14), and the remote processing device is a surgical hub, e.g. surgical hub 1953 (FIG. 13). In one aspect, the control system is the master and the remote processing device is the follower during distributed processing as described in connection to FIGS. 15 and 16. For example, the control system receives surgical data and transmits some of the data to the remote processing device along with a synchronization feature. The synchronization feature is used to prevent out of sequence combinations of results or processes feeding information back to the device control. Thus, the synchronization between the processors can prevent mismatch or loss of data, data overlap, and other errors due to the distributed processing. In one aspect, the synchronization feature enables the system to remove a dataset that is not paired to the current processing, which can prevent mismatching two sets of incompatible or out of temporal sequence data.

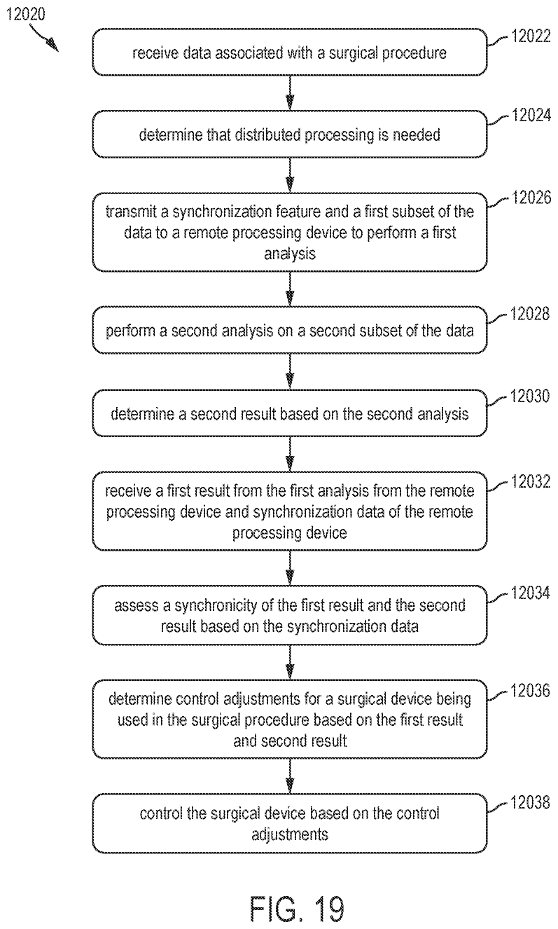

FIG. 19 is a flow diagram depicting a distributed processing method 12020 that can be executed by a plurality of control circuits to concurrently perform data processing tasks, for example. In one aspect, the control circuits include separate processors. In one aspect, the control circuits include a master device, e.g. control circuit 12002 (FIG. 15), and a follower device, e.g. control circuit 12010 (FIG. 15), that simultaneously perform separate data processing tasks to determine parameters required to control a surgical device, e.g. surgical instrument 1010 (FIG. 1) or surgical instrument assembly 200 (FIG. 8). The method 12020 includes receiving 12022, for example by a master control circuit, surgical data associated with a surgical procedure. In one aspect, the surgical data is transmitted from the surgical device.

The method 12020 further includes determining 12024, for example by the master control circuit, that distributed processing of the surgical data is needed. In one aspect, the master control circuit receiving the data determines that distributed processing is needed based on a processing rate of a processor of the control circuit, type, amount, and/or other parameters of the surgical data, and/or a time threshold for the results, for example.

After determining 12024 that distributed processing is needed, the method 12020 can further include connecting the master control circuit with at least one other device to perform the distributed processing. Additionally, the master control circuit can break the data associated with the surgical procedure into subsets for use in the distributed processing. For example, the master control circuit can section out specific tasks, or analyses, and corresponding data, for sending to the at least one other device to perform distributed processing.

The method 12020 further includes transmitting 12026, for example by the master control circuit, a synchronization feature and a first subset of data to a remote processing device to perform a first analysis on the first subset of data. In one aspect, the synchronization feature is a synchronization signal. In one aspect, the first analysis comprises data processing of the first subset of the data. In another aspect, the first analysis includes performing calculations based on the first subset of data to determine a parameter, or multiple parameters, needed by the control circuit to control the surgical device. In yet another aspect, the first analysis includes completing a desired task with the data.

In at least one example, the first subset of data includes image data, and the first analysis includes performing an image processing task on the first subset of data, for example to identify a relevant structure such as an anatomical structure or a component of a surgical device, and to determine, based on the image processing, a parameter, or multiple parameters, required to control the surgical device. Such parameters include, for example, an articulation angle between an end effector and a shaft of the surgical device, staple cartridge configuration of a staple cartridge used in the surgical device, distance from the surgical device to a target object, and/or other relevant parameters.

In at least one example, the first analysis yields a firing force profile of the surgical device (e.g. the firing force applied to jaws 1921, 1931), a closure force profile of the surgical instrument (e.g. the closure force applied to jaws 1921, 1931), a motor (e.g. motor assembly 1939 (FIG. 13), closure motor 603, firing motor 602, or articulation motors 606a, 606b) current profile, and/or a motor velocity profile.

Further to the above, the method 12020 includes performing 12028, by the master control circuit, a second analysis on a second subset of data. In one aspect, the second subset of data is different than the first subset of data. In another aspect, the second subset of data is the same as, or similar to, the first subset of data. The second analysis can be the same or different than the first analysis. In certain instances, the data is not divided, but rather the same analysis is performed 12028 on the same data twice to ensure accuracy.

Furthermore, the first and/or second analyses are based on instructions received by the control circuit along with the data. In another aspect, the first and/or second analyses are automatically selected based on the data received. For example, the first and/or second analyses can be based the type of data received.

Further to the above, the method 12020 includes determining 12030 a second result based on the second analysis. In one aspect, the second result is a parameter, or multiple parameters, required by the control circuit to complete a task. In certain instances, the first subset of data is associated with a first sensor and the second subset of data is associated with a second sensor, and the first and second results cooperatively modify a default parameter of a control algorithm of the surgical device such as, for example, a motor control algorithm.

Further to the above, the method 12020 includes receiving 12032, for example by the master control circuit, a first result from the first analysis from the remote processing device and synchronization data of the remote processing device. The first result could be any of the results described in connection to the first analysis. In one aspect, the first result is a parameter, or multiple parameters, required by the control circuit to complete a task.

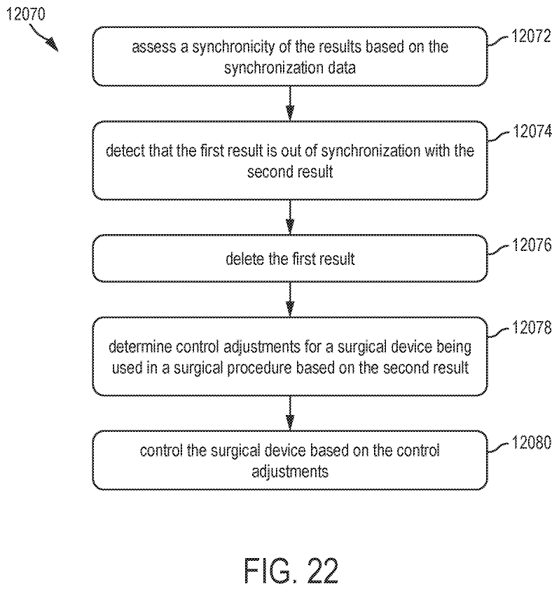

Further to the above, the method 12020 includes assessing 12034, for example by the master control circuit, a synchronicity of the first result and the second result based on the synchronization data. In some aspects, the control circuit evaluates the synchronization data based on a predetermined synchronization tolerance threshold. For example, if the synchronization data is within the predetermined synchronization tolerance threshold, then the control circuit determines that the first result and the second result are in sync, or in proper sequence. Alternatively, if the synchronization data is beyond the predetermined synchronization tolerance threshold, then the control circuit determines that the first result and the second result are out of sync, or out of proper sequence. In this instance, the master control circuit addresses the detected unsynchronicity, for example as described in connection to FIGS. 22-25.

In at least one aspect, when the master control circuit and the remote processing device are synchronized, then their clocks are synchronized. In at least one aspect, the synchronization data is the internal clock time of the remote processing device at transmission of the first result. In this aspect, the control circuit compares the remote processing device's internal clock time to the clock time that the master control circuit received the first result. If the difference between the clock times is larger than a transmission time between the control circuit and the remote processing device, then the results are not synchronized. In one aspect, the transmission time between the master control circuit and the remote processing device can be determined prior to synchronization.

If the results are synchronized, or in proper sequence, then the control circuit can use both results in making decisions and calculations. If the master control circuit determines that the first result and the second result are not synchronized, or out of proper sequence, then the master control circuit does not use both results in making decisions or calculations. Instead, as described in greater detail in connection with FIGS. 22-25, the master control circuit may first address the lack of synchronicity in the data.

Further to the above, the method 12020 includes determining 12036, for example by the master control circuit, control adjustments for a surgical device, e.g. surgical instrument 1010 (FIG. 1) or surgical instrument assembly 200 (FIG. 8), being used in the surgical procedure based on the first result and/or the second result. In one aspect, the control adjustments could be to a motor, e.g. motor assembly 1939 (FIG. 13), firing motor 602, closure motor 603, or articulation motor 606a, 606b, in the surgical device. In another aspect, the control adjustments could be to a power supplied to the surgical device. Further to the above, the method 12020 includes controlling 12038, for example by the master control circuit, the surgical device based on the control adjustments.

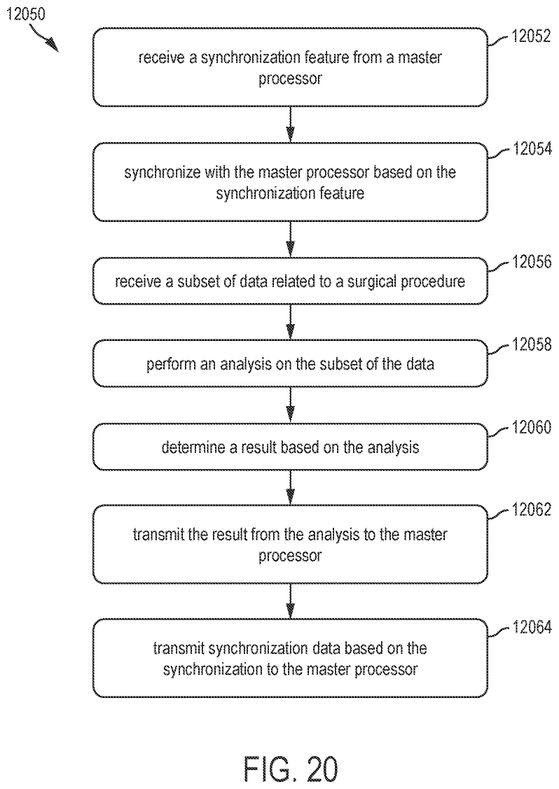

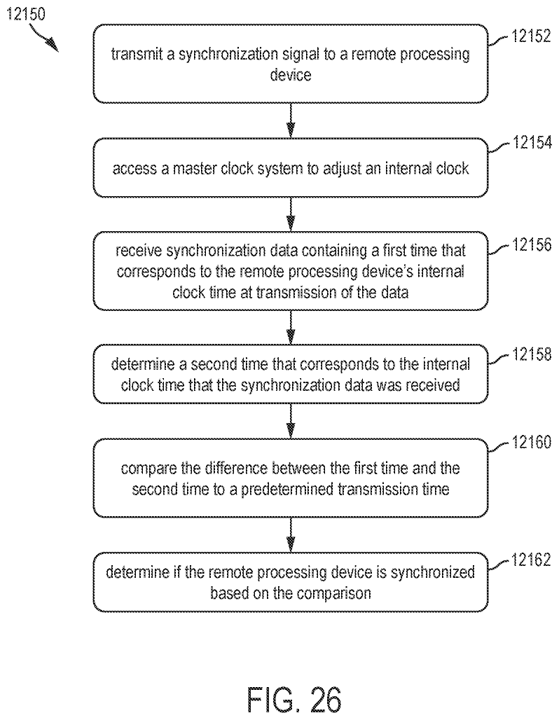

FIG. 20 is a flow diagram of a distributed processing method 12050 that can be executed by a plurality of control circuits to concurrently perform data processing tasks, for example. In one aspect, the control circuits include separate processors. In one aspect, the control circuits include a master device, e.g. control circuit 12002 (FIG. 15), and a follower device, e.g. control circuit 12010 (FIG. 15), that simultaneously performs separate data processing tasks to determine parameters required to control a surgical device, e.g. surgical instrument 1010 (FIG. 1) or surgical instrument assembly 200 (FIG. 8). The method 12050 includes receiving 12052, for example by a follower control circuit, a synchronization feature from another control circuit. In one aspect, the synchronization feature is transmitted from a master control circuit, e.g. control circuit 12002 (FIG. 15). In one aspect, the synchronization feature is a synchronization pulse or signal. The method 12050 further includes synchronizing 12054, for example by the follower control circuit, with the other control circuit, for example the master control circuit, based on the synchronization feature. For example, the follower control circuit can use the synchronization feature to synchronize the follower control circuit with the master control circuit. This can be done by a variety of methods that will be described in more detail in connection to FIGS. 26-28.

Further to the above, the method 12050 includes receiving 12056, for example by the follower control circuit, a subset of data related to a surgical procedure, performing 12058, for example by the follower control circuit, an analysis on the subset of the data, and determining 12060, for example by the follower control circuit, a result based on the analysis, as described in connection to FIG. 19.

Further to the above, the method 12050 includes transmitting 12062, for example by the follower control circuit, the result from the analysis to the other control circuit, for example the master control circuit. The method 12050 further includes transmitting 12064, for example by the follower control circuit, synchronization data based on the synchronization. In one aspect, the synchronization data is generated through the synchronization of the follower control circuit with the master control circuit. In at least one aspect, the synchronization data is the internal clock time of the follower control circuit at transmission of the result.

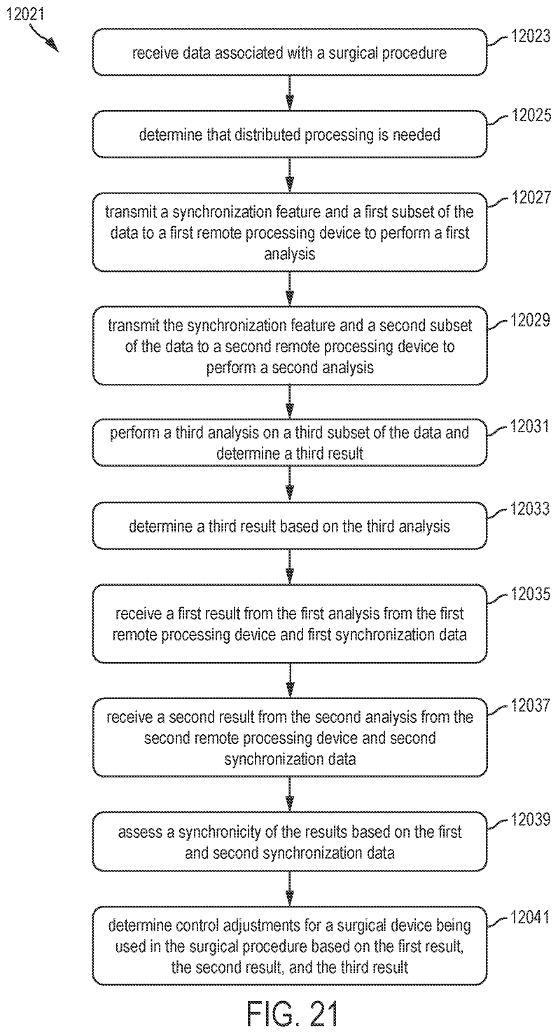

FIG. 19, in one aspect, describes a distributed control process between one master device and one follower device. However, there can be any number of follower devices. FIG. 21 is a flow diagram of a distributed control processing method 12021 that can be executed by a plurality of control circuits to concurrently perform data processing tasks, for example. In one aspect, the control circuits include separate processors. In one aspect, the control circuits include a master device, e.g. control circuit 12002 (FIG. 16), and follower devices, e.g. control circuits 12010, 12011 (FIG. 16), that simultaneously perform separate data processing tasks to determine parameters required to control a surgical device, e.g. surgical instrument 1010 (FIG. 1) or surgical instrument assembly 200 (FIG. 8). The method 12021 further includes receiving 12023, for example by a master control circuit, surgical data associated with a surgical procedure. The method 12021 further includes determining 12025, for example by the master control circuit, that distributed processing of the surgical data is needed, as described in connection with the process of FIG. 19. The method 12021 further includes transmitting 12027, for example by the master control circuit, a synchronization feature and a first subset of data to a first remote processing device to perform a first analysis on the first subset of data, as described in connection with the methods 12020, 12050 of FIGS. 19 and 20.

Further to the above, the method 12021 includes transmitting 12029, by the master control circuit, the synchronization feature and a second subset of data to a second remote processing device to perform a second analysis on the second subset of data. In some aspects, the synchronization feature is the same or similar to the synchronization feature transmitted to the first remote processing device. In one aspect, the second subset of the data is different than the first subset of the data. In another aspect, the second subset of the data is the same as the first subset of the data. The second subset of data and the second analysis could be any data or analysis as described in connection with the methods 12020, 12050 of FIGS. 19 and 20.

Further to the above, the method 12021 includes performing 12031, by the master control circuit, a third analysis on a third subset of the data. In one aspect, the third subset of the data is different than the first subset of the data and/or the second subset of the data. In another aspect, the third subset of the data is the same as the first subset of the data and/or the second subset of the data. The third subset of the data and the third analysis could be any data or analysis as described in connection with the methods 12020, 12050 of FIGS. 19 and 20.

Further to the above, the method 12021 includes determining 12033, by the master control circuit, a third result based on the third analysis. The third result could be any result as described in connection with the methods 12020, 12050 of FIGS. 19 and 20. For example, the third result could be a parameter, or multiple parameters, required by the control circuit to complete a task.

Further to the above, the method 12021 includes receiving 12035, by the master control circuit, a first result from the first analysis from the first remote processing device and first synchronization data of the first remote processing device. Further to the above, the method 12021 includes receiving 12037, by the master control circuit, a second result from the second analysis from the second remote processing device and second synchronization data of the second remote processing device. The first result, first synchronization data, second result, and second synchronization data could be any result or synchronization data as described in connection with the methods 12020, 12050 of FIGS. 19 and 20. For example, the first result and the second result could be parameters that are required by the control circuit to complete a task.

Further to the above, the method 12021 includes assessing 12039, by the master control circuit, a synchronicity of the first result, the second result, and the third result based on the first synchronization data and the second synchronization data. The process of assessing the synchronization can be similar to that described in connection with the process of FIG. 19. In one aspect, the synchronicity between the two remote processing devices and the master device is assessed based on a predetermined synchronization tolerance threshold. For example, the master control circuit could evaluate the first synchronization data and the second synchronization data based on the predetermined threshold, as described in greater detail in connection with FIG. 19. If the first synchronization data and second synchronization data are within the predetermined synchronization tolerance threshold, then the control circuit can determine that the first result, the second result, and the third result are synchronized. This allows the master control circuit to use these results in making decisions and calculations. If the master control circuit determines that the results are not synchronized, then the master control circuit cannot combine and use the results. In this instance, the master control circuit addresses the detected unsynchronicity, for example as described in connection to FIGS. 22-25. While the methods described in connection to FIG. 22-25 describe methods to handle unsynchronized results for one remote processing device and a master device, they can be expanded to include any number of remote processing devices.

Further to the above, the method 12021 includes determining 12041, by the master control circuit, control adjustments for a surgical device, e.g. surgical instrument 1010 (FIG. 1) or surgical instrument assembly 200 (FIG. 8), being used in the surgical procedure based on the first result, the second result, and the third result. In one aspect, the control adjustments could be to a motor, e.g. motor assembly 1939 (FIG. 13), firing motor 602, closure motor 603, articulation motor 606a, 606b, in the surgical device. In another aspect, the control adjustments could be to a power source supplying power to the surgical device. After the control adjustments are made, the master control circuit can control the surgical device based on the control adjustments.

There are a variety of approaches to handle unsynchronized data. For example, if a control circuit detects a loss of synchronization, the control circuit could drop both parts of the un-synchronized result and look for the next received set to be synchronized. Alternatively, the control circuit could drop an unsynchronized result and either does not use it or substitutes it with a nominal, pre-determined value, or with the last valid result. However, in most instances, the substituted value could only be used for a limited time before the system would shut down for safety concerns due to not having updated results. FIGS. 22-25 describe some of these approaches.

FIG. 22 is a flow diagram depicting a method 12070 for a control circuit to execute when results of a distributed processing method are out of synchronization. In one aspect, the control circuit includes a separate processor. In one aspect, the control circuit is a master device, e.g. control circuit 12002 (FIG. 15). Specifically, the method 12070 includes deleting the out of sync result and determining control adjustments with the remaining result. In the illustrated example, the method 12070 includes assessing 12072, for example by a master control circuit, a synchronicity of the results based on the synchronization data, as described in connection with FIG. 19. The method 12070 further includes detecting 12074, for example by the master control circuit, that the first result is out of synchronization with the second result. The method 12070 further includes deleting 12076 is the first result. The method 12070 further includes determining 12078 control adjustments for a surgical device, e.g. surgical instrument 1010 (FIG. 1) or surgical instrument assembly 200 (FIG. 8), being used in a surgical procedure based on the second result, but not the deleted first results. The control adjustments can be any of the control adjustments described in connection to FIG. 19. The method 12070 may further include controlling 12080 the surgical device based on the control adjustments. In some aspects, if the master control circuit and the follower control circuit are not able to resynchronize after a predetermined time period from the detection 12074 of the out of sync status, then the master control circuit can appropriate safety measure such as, for example, shutting down the surgical device and/or alerting a user through any suitable user interface.

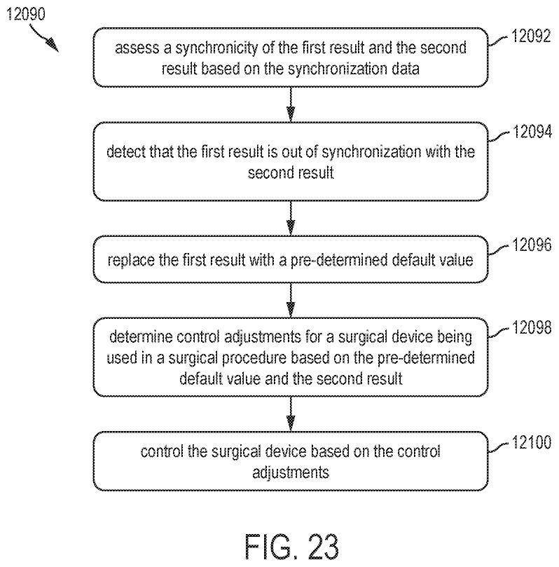

FIG. 23 is a flow diagram depicting a method 12090 for a control circuit to execute when results of a distributed processing method are out of synchronization. In one aspect, the control circuit includes a separate processor. In one aspect, the control circuit is a master device, e.g. control circuit 12002 (FIG. 15). The method 12090 includes assessing 12092 a synchronicity of the results based on the synchronization data, as described in connection with FIG. 19. The method 12090 further includes detecting 12094 that the first result is out of synchronization with the second result. The method 12090 further includes replacing 12096 the out of synchronization result, which in this instance is the first result, with a predetermined default value. In at least one aspect, a predetermined value in accordance with predetermined safety practices is used in place of the first result. The method 12090 further includes determining 12098 control adjustments for a surgical device, e.g. surgical instrument 1010 (FIG. 1) or surgical instrument assembly 200 (FIG. 8), being used in a surgical procedure based on the pre-determined default value and the second result. The control adjustments can be any of the control adjustments described in connection to FIG. 19. The method 12090 may further include controlling 12100, for example by the master control circuit, the surgical device based on the control adjustments. In some aspects, if the master control circuit and the follower control circuit are not able to resynchronize after a predetermined time period from the detection 12094 of the out of sync status, then the master control circuit can appropriate safety measure such as, for example, shutting down the surgical device and/or alerting a user through any suitable user interface.

In another aspect, the method 12090 described in connection to FIG. 23 could be performed by replacing both the first result and the second result by default values, when either the first result or the second result is out of synchronization. Then the control circuit could determine control adjustments for the surgical device being used in the surgical procedure based on a first default value and a second default value. For example, the first default value could replace the first result and the second default value could replace the second result.

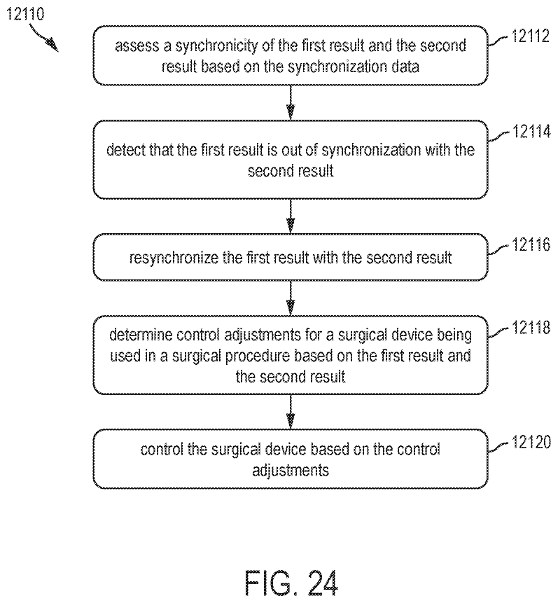

FIG. 24 is a flow diagram depicting a method 12110 for a control circuit to execute when results of a distributed processing method are out of synchronization. In one aspect, the control circuit includes a separate processor. In one aspect, the control circuit is a master device, e.g. control circuit 12002 (FIG. 15). Specifically, the flow diagram illustrates resyncing the out of sync result. The method 12110 includes assessing 12112, for example by a master control circuit, a synchronicity of the results based on the synchronization data, as described in connection with FIG. 19. The method 12110 includes detecting 12114 that the first result is out of synchronization with the second result. The method 12110 further includes resynchronizing 12116 the first result with the second result. In at least one aspect, the master control circuit resynchronizes the first result and the second result based on the synchronization feature and/or synchronization data. For example, in one aspect, the master control circuit could determine an amount of time that the remote processing device drifted out of synchronization based on the synchronization data and resynchronize the first result based on the determined amount of time. The method 12110 further includes determining 12118 control adjustments for a surgical device, and optionally controlling 12120 the surgical device based on the control adjustments, as described in connection with FIG. 19.

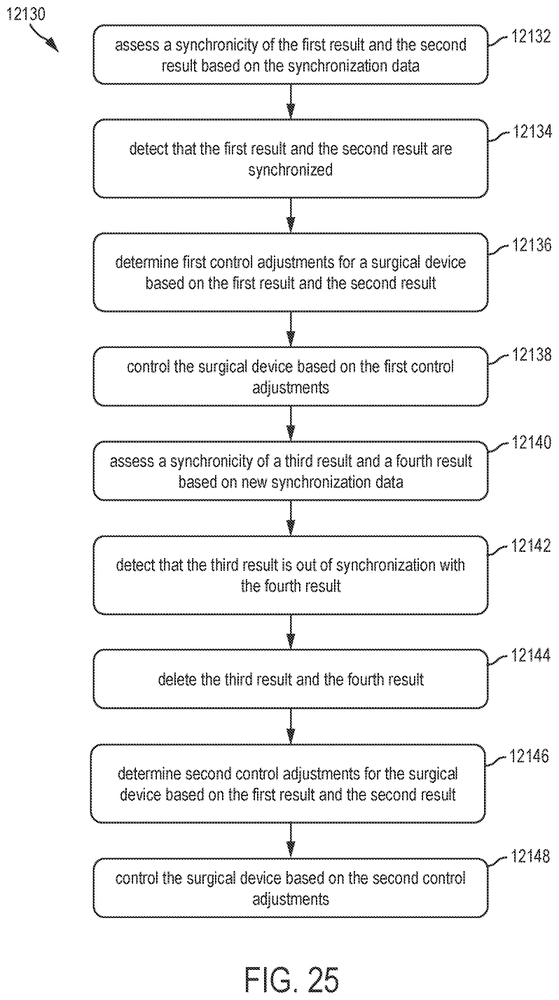

FIG. 25 is a flow diagram depicting a method 12130 for a control circuit to execute when results of a distributed processing method are out of synchronization. In one aspect, the control circuit includes a separate processor. In one aspect, the control circuit is a master device, e.g. control circuit 12002 (FIG. 15). Specifically, the method 12130 uses a previously synchronized result in substitute of the current unsynchronized results. The method 12130 includes assessing 12132 a synchronicity of the results based on the synchronization data, as described in connection with FIG. 19. The method 12130 further includes detecting 12134 that the first result and the second result are synchronized, determining 12136 control adjustments for a surgical device based on the first and second results, and controlling 12138 the surgical device based on the first control adjustments.

Later in the surgical procedure the control circuit, for example by the master control circuit, receives new data associated with the surgical procedure. The control circuit, for example by the master control circuit, performs distributed processing with the new data, as described in connection to FIG. 19, and the control circuit receives, for example by the master control circuit, a third result and calculates a fourth result.

The method 12130 further includes assessing 12140 a synchronicity of third and fourth results of analyses performed on new data subjected to distribute processing, wherein the synchronicity of the third and fourth results is based on new synchronization data. The method 12130 further includes detecting 12142 that the third result is out of synchronization with the fourth result. The method 12130 further includes deleting 12144 the third result and the fourth result, and determining 12146 second control adjustments for the surgical device based on the first result and the second result. Stated another way, the master control circuit deletes the current results if they are not synchronized and determines control adjustments for the surgical device based on the previous results that were synchronized. In some aspects, the second control adjustments can be the same or similar to the first control adjustments. In other aspects, the second control adjustments can be different from the first control adjustments. The method 12130 further includes controlling 12148 the surgical device based on the second control adjustments.

Various methods and systems of the present disclosure utilize the transmission of synchronization features and/or retrieval of synchronization data to ensure synchronicity of surgical data subjected to distributed processing by multiple processors, e.g. processors 12004, 12012, 12013, to prevent the mismatch or loss of results. There are many approaches to synchronizing processors. In various examples, communication protocols are utilized to insure synchronization and minimizing of data loss. The processors can use these communication protocols when sending data between the processors to help keep the processors synchronized. Non-limiting examples communication protocols include Integrating Simple Network Time Protocol (SNTP), Network Time Protocol (NTP), and Precision Time Protocol (PTP). These protocols allow for multiple embedded systems to be synchronized.

In some aspects, multiple processors need to be synchronized to complete certain tasks. For example, a surgical device, e.g. surgical instrument 1010 (FIG. 1) or surgical instrument assembly 200 (FIG. 8), can include multiple motors, where each motor has a motor control circuit that controls that motor. In one aspect, the surgical device includes a main control circuit that is communicably coupled to the motor control circuits to transmit motor control signals to the motor control circuits for multi-motor control. In various aspects, the main control circuit and the motor control circuits are synchronized for smooth multi-motor control by the main control circuit.

FIG. 26 is a flow diagram depicting a method 12150 for a control circuit to execute to synchronize with another control circuit, or a plurality of control circuits. In one aspect, the control circuits include separate processors. In one aspect, the control circuits include a master device, e.g. control circuit 12002 (FIG. 15) and a follower device, e.g. control circuit 12010 (FIG. 15). In various aspects, a master clock can be accessible by all the processors, e.g. processors 12004, 12012, 12013, to adjust and sync

The method 12150 includes transmitting 12152 a synchronization signal to a remote processing device, and accessing 12154 a master clock to adjust its internal clock to match the master clock. In at least one aspect, the master device and the remote processing device both have access to the master clock, which allows the master device and the remote processing device to synchronize their clocks to the master clock. In an alternative aspect, the master device could contain the master clock. In this aspect, the follower device would synchronize directly to the master device clock.

Further to the above, the method 12150 includes receiving 12156, by the master control circuit, synchronization data containing a first time that corresponds to the remote processing device's internal clock time at transmission of the synchronization data. The method 12170 further includes determining 12158 a second time that corresponds to the internal clock time at which the synchronization data was received. To assess the synchronization between the devices, the method 12150 further includes comparing 12160 the difference between the first time and the second time to a predetermined transmission time. In at least one aspect, the predetermined transmission time is determined prior to performing any distributed processing or synchronizing. In some aspects, the predetermined transmission time is the time that it takes for the remote processing device to transmit a message to the master device. The method 12150 further includes determining 12162 if the remote processing device is synchronized. In one aspect, if the difference between the first time and the second time is below the predetermined transmission time, then the master device and the remote processing device are determined to be synchronized. If not, then the master device will transmit another synchronization signal and repeat the synchronicity testing until synchronized.

One method to maintain synchronization between processors, or control circuits, is to send a synchronization byte pattern, or timing mark, from the master processor, e.g. processor 12004, to the follower processors, e.g. processors 12012, 12013. For example, the pattern could be a signal that is a sequence of ones and zeroes which includes a timing element. The follower processors all check the byte pattern to verify that no packets have been lost and use the timing element to help assure timing is maintained. For example, if the byte pattern received by the follower processor is not the expected byte pattern, then the follower processor determines that it is out of synchronization with the master processor. Additionally, if a packet has been determined to be lost, then the follower processor can also determine that it is out of synchronization with the master processor. In either case, the follower processor can attempt to resynchronize with the master processor as described above in connection to FIG. 26.

The latency between two or more synchronized processors can be characterized for fine tuning their synchronization. One method is to use an electric sync with a hardware clock. The hardware clock could have a crystal oscillator that is compensated for temperature and humidity. This approach can produce a very staple signal. This signal is used to synchronize the processors, e.g. processors 12004, 12012, 12013, and measure any drift by any of the processors. In one aspect, the electric sync is coupled to the processors 12004, 12012, 12013 such that it can transmit a signal to the processors. In one aspect, the signal is transmitted to each processor at the same time and the processors sync their internal clocks based on the received signal. At a predetermined set time interval, a signal could be sent from the electric sync and received by the processors. In one aspect, the processors use the received signal to resync and record any drift in time. For example, if the processor knows that the predetermined resync time interval is 10 minutes and it receives the sync signal from the electric sync at 9 minutes and 59 seconds, the processor knows that it drifted by 1 second, for example. In certain instances, drift data can be transmitted back to the master control circuit along with the result of a data analysis. The master control circuit can resynchronize the result with other results from data subjected to distributed processing on multiple devices based on the drift data.

Another method to characterize the latency between processors is to use a software clock approach. This is a process where the master device, e.g. control circuit 12002, can transmit a sync pulse to each of the follower devices, e.g. control circuits 12010, 12011, that have already synced with the master device. In at least one aspect, the master device is a surgical hub, e.g. surgical hub 1953 (FIG. 13), and the follower device is a remote processing device. The pulse can be used to check the latency of the follower device and adjust the follower device to be in better synchronization with the master device. The master device, e.g. control circuit 12002, can transmit a message to the follower devices, e.g. control circuits 12010, 12011, that includes a fixed frequency. Later the master device transmits a signal at the fixed frequency to each follower device. The follower devices can measure the actual frequency coming from the master device and adjust their clock signals accordingly to align with the master device.

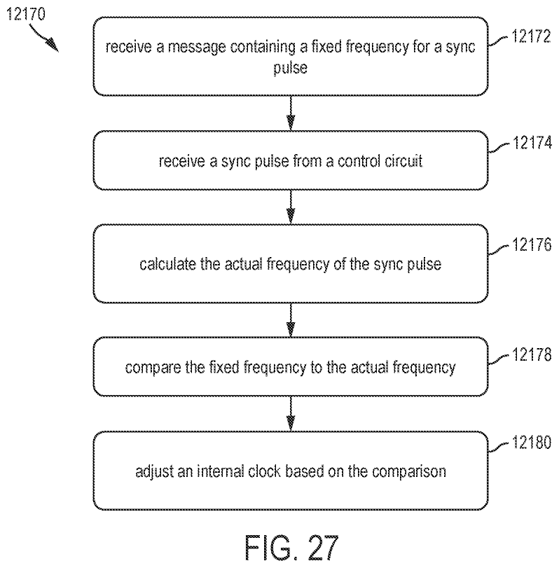

FIG. 27 is a flow diagram depicting a method 12170 that can be executed by a control circuit to characterize the latency of another synchronized control circuit. In one aspect, the control circuits include separate processors. In one aspect, the control circuits include a master device, e.g. control circuit 12002 (FIG. 16), and a follower device, e.g. control circuits 12010, 12011 (FIG. 16). The method 12170 includes receiving 12172, for example by the follower device, a message containing a fixed frequency for a sync pulse. The method 12170 further includes receiving 12174, for example by the follower control circuit, a sync pulse signal from a control circuit, for example from the master control circuit. In at least one aspect, the follower control circuit stores the received sync pulse signal in a memory, e.g. memory 12014 or memory 12015. The method 12170 further includes calculating 12176, for example by the follower control circuit, the actual frequency of the sync pulse signal. The method 12170 further includes comparing 12178, for example by the follower control circuit, the received fixed frequency to the calculated actual frequency. The method 12170 further includes adjusting 12180, for example by the follower control circuit, the internal clock based on the comparison. For example, in one aspect, the follower control circuit could adjust its internal clock if there was a difference between the actual frequency measured and the fixed frequency that it received. In an alternative aspect, if there was no difference between the actual frequency and the fixed frequency, then the follower device is already synchronized with the master device.

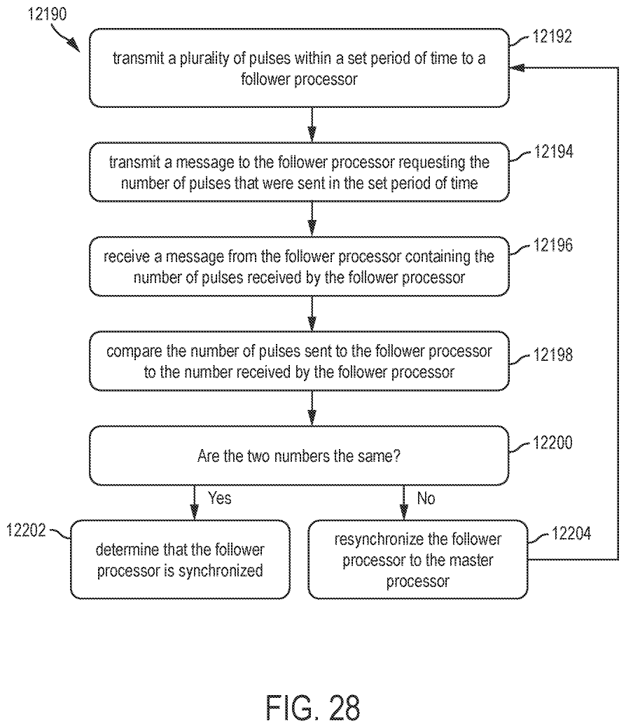

FIG. 28 is a flow diagram depicting a method 12190 that can be executed by a control circuit to characterize the latency of another synchronized control circuit. In one aspect, the control circuits include separate processors. In one aspect, the control circuits include a master device, e.g. control circuit 12002 (FIG. 16), and a follower device, e.g. control circuit 12010 (FIG. 16) or control circuit 12011 (FIG. 16). The master device could periodically send out a specific number of pulses to a follower device and then request the number of pulses that have occurred within a set time period from the follower device. If the follower's counted number of pulses does not agree with the number sent by the master device, the master device could try to re-sync the follower device to better align the master and follower devices. This can help prevent data loss (loss of packets) or timings being slightly off of each other.

The method 12190 includes transmitting 12192, for example by the master control circuit, a plurality of pulses within a set period of time to another control circuit, for example the follower control circuit. The method 12190 further includes transmitting 12194, for example by the master control circuit, a message to the other control circuit, for example the follower control circuit, requesting the number of pulses that were sent in a set period of time. The method 12190 further includes receiving 12196, for example by the master control circuit, a message from the other control circuit, for example the follower control circuit, containing the number of pulses received by the other control circuit, for example the follower control circuit, during the requested time. The method 12190 further includes comparing 12198, for example by the master control circuit, the number of pulses sent to the other control circuit, for example the follower control circuit, to the number received by the other control circuit, for example the follower control circuit. The method 12190 further includes determining 12200, for example by the master control circuit, if the two numbers are the same or not. If the two numbers are the same, then the method proceeds down the “Yes” branch, where the method further includes determining 12202, for example by the master control circuit, that the other control circuit, for example the follower control circuit, is synchronized. If the two numbers are not the same, then the method proceeds down the “No” branch, where the method further includes resynchronizing 12204, for example by the master control circuit, the other control circuit, for example through the method described in connection to FIG. 26. After the other control circuit, for example the follower control circuit, is resynchronized, the method proceeds to repeat 12192-12200 to ensure that the other control circuit synchronized properly.

The transmissions between a master control circuit, e.g. control circuit 12002, and the follower control circuit, e.g. one of control circuits 12010, 12011, during distributed processing, as described in connection to FIGS. 19 and 20, can have a specific packet that includes time stamps associated with sending time (message assembly time and assembly time of the data) and transmission time. In one aspect, the master control circuit receives this information from the follower control circuit each transmission of the follower control circuit. The master control circuit uses this information to track the time the follower control circuit spends on the data processing. From this amount of time, the master control circuit determines more control circuits need added to the distributed processing to complete tasks on time. In at least one aspect, the master control circuit can compare the amount of time the follower control circuit took to process the data to a predetermined process time. For example, if the time the follower control circuit took to process the data exceeds the predetermined process time, then the master control circuit can determine that more follower control circuits need added to the distributed processing.

In one aspect, the transmission of the data from the follower control circuit to the master control circuit could be delayed due to it being a lower priority than another function the follower control circuit is performing. This delay time can be captured and transmitted when allowed to the other control circuits, e.g. the master control circuit. This information can allow the master control circuit to determine where and why a delay occurred. In some aspects, the master control circuit could communicably couple to another follower control circuit if the delays are too large.

Multiple timestamps can be used by the master control circuit to make decisions about the distributed processing. The propagation time can be tracked. This is a time period represented by the time required for the master control circuit to send a data packet to the time the follower initially receives the data packet. The reception time, or receive time, is a time period from the timestamp the message was first received by the follower control circuit to the timestamp the message was processed. The decoding time is a timestamp to indicate a time period required to unpack the data. These different time periods allow the master control circuit to track what each follower control circuit is doing and detect any delays that are occurring. This information is used to keep the control circuits in sync and completing tasks in a timely manner.

In some situations it can be beneficial to have a subsystem post processor adaptation of motor control. This brings an additional level of control to the motor control system. This concept is implemented after the main control circuit, e.g. control circuit 1932 (FIG. 13), surgical hub 1953 (FIG. 13), or control circuit 620 (FIG. 14), to give an additional layer of control and/or safety to the system. The subsystem could be electrical and/or mechanical in nature. For example, the subsystem could provide physical and/or electrical adaptations after a motor command signal. In some aspects, the subsystem is sampling at a much higher rate to see situations that the main control circuit missed while making decisions with current data.

Some surgical devices, e.g. surgical instrument 1010 (FIG. 1) or surgical instrument assembly 200 (FIG. 8), can have onboard local processing. The on-device processor monitors the voltage and/or current applied to a motor, e.g. motor assembly 1939 (FIG. 13), closure motor 603, firing motor 602, or and articulation motors 606a, 606b, as well as at least one other electrical sensor coupled to the drive train. These monitored parameters can be processed locally and used to adjust electrical controls to a controller, e.g. control circuit 1932 (FIG. 13), surgical hub 1953 (FIG. 13), or control circuit 620 (FIG. 14), of the motor. In some aspects, each motor control command can be accompanied by a max current threshold. In at least one aspect, hall effect switches are placed on control locations, e.g. an articulation joint between a shaft of the surgical device and an end effector of the surgical device, and used to initiate different commands from the motor through the controller. In at least one aspect, the controller controls the motor through a proportional-integral-derivative control of a pulse-width modulation duty cycle based on velocity and position monitoring. The data is all processed on the device on the main processors or within the motor controller sub-processor to adjust operations of the device.

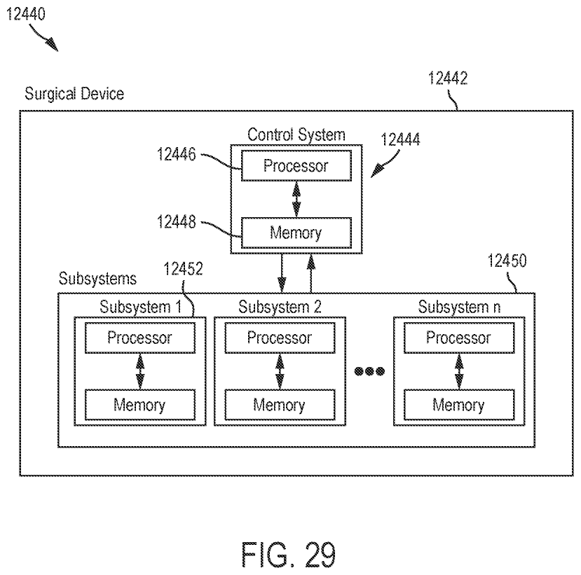

FIG. 29 illustrates a diagram 12440 of a surgical system 12442, e.g. surgical instrument 1010 (FIG. 1) or surgical instrument assembly 200 (FIG. 8), that has a control circuit 12444, e.g. control circuit 1932 (FIG. 13), surgical hub 1953 (FIG. 13), or control circuit 620 (FIG. 14). The surgical system 12442 includes a processor 12446 coupled to a memory 12448. The surgical system 12442 can include a plurality of subsystems 12450 that can be communicably coupled to the control circuit 12444 or, alternatively, operate independently of the control circuit 12444. The subsystems 12450 are configured to perform specialized adjustments outside, or beyond, the control of the control circuit 12444. In one aspect, the subsystems 12450 include an electrical subsystem 12452 that can make adaptations to a motor control command after the processor 12446 transmits a command signal to a motor, e.g. motor assembly 1939 (FIG. 13), closure motor 603, firing motor 602, or and articulation motors 606a, 606b. In one aspect, subsystem control circuit 12452 modifies a motor drive signal set by the control circuit 12444, wherein the modification is beyond the control of the control circuit 12444.

The electrical subsystem 12450 brings an additional level of control of the motor, and may interact with the motor separate from the control circuit 12444. In one aspects, the electrical subsystem 12450 receives an input signal from dedicated sensors, separate from the sensors 1938 that are communicably coupled to the control circuit 12444. The electrical subsystem 12450 may perform an adjustment of the motor drive signal set by the control circuit 12444 based on the input signal from the dedicate sensors. The adjustments can be performed independent of, or beyond the control of, the control circuit 12444.

In at least one aspect, the subsystem 12452 receives a signal indicative of a parameter of the surgical device from a sensor, and limits the power (voltage or current) available to the motor based on the sensor value. In one aspect, the subsystem 12452 includes an electrical element diverts a portion of the power through a non-functional circuit branch effectively limiting the overall power available to the motor. In one aspect, the sensor signal could be indicative of an articulation angle between the shaft of the surgical device and the end effector of the surgical device.

There are a variety of methods that could be employed by the electrical subsystem to control or limit the available power to the motor, e.g. motor assembly 1939 (FIG. 13), firing motor 602, closure motor 603, articulation motor 606a, 606b. In one aspect, a subsystem 12452 receives an operational system positional location and changes the power available to the motor based on the location. In another aspect, a subsystem control circuit 12452 receives a detected an input signal indicative of a load on the system, e.g. a force on the motor or a force on the end effector of the surgical device, and limits the current available to the motor based on the detected load. In at least one aspect, the subsystem control circuit 12452 uses the load as an input to control the available current to the motor. This process can help prevent damage to the motor assembly 1939 and/or the drive assembly 1941. In one aspect, a subsystem 12452 directs the current to the motor through an in-line power resistor to limit the power to the motor. In another aspect, a subsystem 12452 regulates a high powered voltage regulator that solely supplies power to the motor. In this aspect, a subsystem 12452 pulse-width modulates the regulator to supply a standard signal to the motor control circuitry based on the sensor signal.

While the illustrated example shows multiple subsystems 12452 with identical components, it is understood that various subsystems 12452 that perform dedicated functions downstream from the control circuit 12452 can include any suitable circuit components, as previously described, to perform their specific functions.

In certain aspects, a subsystem 12452 directly alters the commands from the control circuit 12444. For example, the control circuit 12444 could place a 50% duty cycle to an H-Bridge circuit of the motor assembly 1939. In such aspects, the subsystem 12452 intercepts the signal the control circuit 12444 generates for the H-Bridge circuit, beyond control of, or even awareness of, the control circuit 12444. In some aspects, the subsystem 12452 can perform sampling tasks, for example based on input signals from one or more sensors, at a much higher rate than that available to the control circuit 1244 to address situations that the control circuit 12444 missed while making calculations with current data. In one exemplification, the control circuit 12444 transmits a command of a duty cycle of 80% with an over current protection flag; however, the subsystem 12452 overrides that command due to a higher risk level event which prohibits the increase.

In at least one aspect, the subsystem 12452 interacts directly with the motor. In other aspects, a subsystem 12452 includes mechanical and/or electrical components, in addition to, or instead of, the processor and memory components, which bring an additional level of control to the motor control system. Such components include, for example, a system dampener. In some aspects, the subsystem 12452 comprises a sensing array. In one aspect, a subsystem 12452 includes mechanical and/or electrical components that adjust motor parameters based on input signals from the sensor array. In at least one aspect, the sensor signal is indicative of a parameter of the surgical device. In at least one aspect, a portion of the force or stroke of the motor is compensated for by an adjustment of the mechanical and/or electrical components. For example, the subsystem 12452 may include a system dampener controllable to provide a rigid response or an adjustable response based on the detected sensed parameter.

In some aspects, the motor is coupled to a gearbox and the subsystem 12452 adjusts the motor gearbox based on the sensed parameter. For example, the subsystem 12452 can shift the gears within the gearbox to change speeds and torques of the motor assembly 1939. In another aspect, the subsystem could make motor magnetic adjustments. In at least one aspect, the motor includes internal permanent magnets and the subsystem 12452 adjusts the magnetics of the motor internal permanent magnets based on the sensed parameter, which adjusts the motor output as desired. In another aspect, the motor includes a shaft that is coupled to a linear brake and the subsystem control circuit 12452 adjusts the pressure brake applied to the motor based on the sensed parameter. In another aspect, the motor includes a drive member that is coupled to a closed loop hydraulic or pneumatic dampener.

The closed loop hydraulic or pneumatic dampener is coupled to an electrical release valve that is configured to be controlled based on the load applied to the drive member, e.g. a force on the motor or a force on the end effector of the surgical device. In one aspect, the subsystem 2452 is coupled to the electrical release valve and transmits a signal to control the electrical release valve based on the sensed parameter. This enables a primary resisting spine of the pneumatic dampener to expand or contract under the load applied by the drive member of the motor. If the valve is kept closed the dampener would have a first length of the restraining system. If the valve is opened it would allow the dampener to elongate or contract thereby adjusting the effective forces applied to an end-effector, e.g. end effector 130 (FIG. 6), end effector 230 (FIG. 8), or end effector 1940 (FIG. 13). The subsystem 12452 controls the amount of opening, which controls the rate of change of the dampener. In one aspect, the subsystem 12452 transmits a signal to the electrical release valve that opens the electrical release valve. Alternatively, the subsystem 12452 could open and close the value in a pulsing manner to control the rate of change of the restraint.

Distributed processing, as described in connection with FIGS. 15 and 16, can be useful in effectively analyzing large amounts of data under tight time constraints. As described elsewhere herein in greater detail, a control circuit, e.g. control circuit 12002 or control circuit 12400, can access additional processing resources through distributed processing to efficiently perform multiple tasks during a surgical procedure. In some aspects, the control circuit employs a prioritization algorithm and/or a delegation algorithm to control aspects of the distributed processing. In one aspect, the prioritization algorithm, when executed by the control circuit, selects certain tasks for local processing and other tasks for external processing. In one aspect, the prioritization algorithm, when executed by the control circuit, assigns priority levels to upcoming tasks to prioritize mission critical tasks and/or time critical feedbacks based on risk levels associated with such tasks, for example.

Further to the above, in certain aspects, the prioritization algorithm, when executed by the control circuit, prioritizes decision making, or signal processing, required for local primary control of actuators, e.g. motor assembly 1939 (FIG. 13), firing motor 602, closure motor 603, articulation motor 606a, 606b, of a surgical device, e.g. surgical instrument 1010 (FIG. 1) or surgical instrument assembly 200 (FIG. 8), over other secondary, commentary, or non-real-time, decision making, or signal processing. In one aspect, the decision making, or signal processing, that is required for local primary control of actuators is performed locally on processor 12406 (FIG. 18) and the other secondary, commentary, or non-real-time the decision making, or signal processing, are delegated, through distributed processing, one or more of the processors 12402, 12414, 12420 (FIG. 18). The secondary, commentary, or non-real-time tasks take advantage of distributed processing without compromising the primary tasks.

Further to the above, in some aspects, the prioritization algorithm, when executed by the control circuit, adjusts a default control algorithm for controlling a motor during a tissue-treatment motion, for example, by causing the default control algorithm to bypass a control function that involves a secondary or complimentary signal that is missing, out of sequence, or unsynchronized. In another aspects, the prioritization algorithm causes the default control algorithm to rely instead on locally processed data or a default, or nominal, value or, alternatively, a last received, in sync, value of the secondary signal as actuator controller signals.

In one exemplification, a surgical device, e.g. surgical instrument 1010 (FIG. 1) or surgical instrument assembly 200 (FIG. 8), includes a motor, e.g. motor assembly 1939 (FIG. 13), firing motor 602, closure motor 603, articulation motor 606a, 606b, a speed sensor that monitors the speed of the motor, and a control circuit, e.g. control circuit 1932 (FIG. 13), surgical hub 1953 (FIG. 13), or control circuit 620 (FIG. 14). The control circuit is coupled to the motor and the speed sensor. The control circuit is continually receiving a signal from the speed sensor. If the signal from the speed sensor stops, then the control circuit replaces the sensor signal with a default or an estimation to continue controlling the motor. The control circuit monitors the signal from the sensor to determine if the sensor is operational. If a predetermined amount of time is exceeded without receiving a signal from the sensor, then control circuit shuts down the motor for safety, and/or issues an alert through a user interface. In such exemplification, the default parameter can only be used or substituted for a set amount of time.

In at least one aspect, there could be some additional speed sensors, for example, one on the motor itself, a second speed sensor on an output of a gearbox coupled to the motor, and a third speed sensor on a drive bar coupled to the gearbox. In one aspect, the processing of these three signals is done with distributed processing. In this instance, the control circuit continues to make decisions if one or two of these sensors stops sending a signal. As long as not all 3 sensors are “off-line”, the control system continues to function. If all three sensors go “off-line”, then the control circuit responds by shutting down the motor for safety purposes, as described above. In other words, the control circuit executing the prioritization algorithm bypasses a calculation that takes into consideration a missing sensor signal in favor of available sensor signals.

In various aspects, the prioritization algorithm involves a tiered matrix based on task criticality, task urgency, and/or processor capabilities and/or constraints. In at least one aspect, a threshold that determines what is processed locally, remotely, or non-real-time is based on the capacity of the master processor, e.g. processor 12004. This threshold could be some percentage of the max capacity such as, for example, 85%-95% of the max capacity to avoid overloading the processor. In addition to processor capacity, the processor power requirements, temperature/heat generation, or communication can all be requirements used as a part of the differentiating threshold.

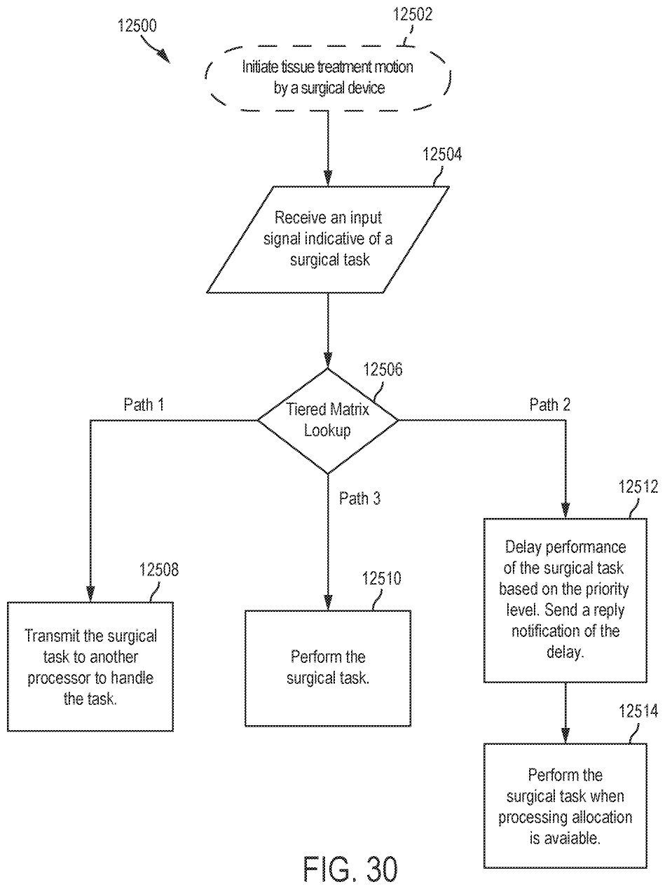

FIG. 30 is a flow diagram depicting a prioritization method 12500 that can be executed by a control circuit for processing tasks based on a tiered matrix. In one aspect, the control circuit includes a master device, e.g. control circuit 12002 (FIG. 15). The method 12500 includes initiating 12502 a tissue-treatment motion by a surgical device. In at least one aspect, the surgical device includes a control, e.g. control circuit 1932 (FIG. 13) or control circuit 620 (FIG. 14). The method 12500 further includes receiving 12504 an input signal indicative of a surgical task to be performed. In at least one aspect, the input signal is received from a surgical hub, e.g. surgical hub 1953 (FIG. 13). For example, the surgical device during a cycle, receives a time critical surgical task from the surgical hub or any sensor element. The method 12500 further includes determining 12506, based on a tiered matrix, for example by the surgical device control circuit, how to address such surgical task. In various instances, the surgical task can be a computational task, a data processing task, or any other task suitable for performance by a processor.

In the illustrated example, the tiered matrix includes three possible paths. It is, however, understood that more or less than three paths can be adopted. Under “Path 1”, the method 12500 includes transmitting 12508, by the surgical device control circuit, the surgical task to another processor. Accordingly, the surgical task is delegated to the other processor. In certain instances, the transmission 12508 can include a feedback request regarding whether the other processor is able to timely perform the surgical task. In one aspects, Path 1 is selected for secondary, commentary, or non-real-time tasks. On the contrary, for primary tasks, Path 2 is selected, where the control circuit locally performs 12510 the surgical task. For Path 3, the method 12500 includes delaying 12512 a performance of the surgical task based on the priority level. In certain instances, a reply notification is issued indicating a decision to locally perform 12514 the task when processing allocation becomes available.

Various aspects of the subject matter described herein are set out in the following examples.

Example 1—A surgical system for use with a surgical device. The surgical system comprises a remote processing device comprising a device control circuit and a surgical hub configured to communicably couple to the remote processing device and the surgical device. The surgical hub comprises a hub control circuit, wherein the hub control circuit and the device control circuit perform distributed processing. The remote processing device control circuit is configured to receive a first subset of data associated with the surgical device from the hub control circuit, receive a synchronization feature associated with the date from the hub control circuit, and perform a first analysis on the first subset of the data. The remote processing device control circuit is further configured to transmit a first result based on the first analysis to the hub control circuit. The first result comprises synchronization data of the remote processing device based on the synchronization feature. The surgical hub control circuit is configured to transmit the synchronization feature to the remote processing device, transmit the first subset of the data to the remote processing device, and perform a second analysis on a second subset of the data. The surgical hub control circuit is further configured to determine a second result based on the second analysis, receive the first result from the remote processing device and synchronization data of the first result, and assess a synchronicity of the first result and the second result based on the synchronization data. The surgical hub control circuit is further configured to determine control adjustments for the surgical device based on at least one of the first result or the second result.

Example 2—The surgical system of Example 1, wherein the synchronization feature is a synchronization signal.

Example 3—The surgical system of Examples 1 or 2, wherein the surgical hub control circuit is further configured to detect that the first result is out of synchronization with the second result, delete the first result, and determine the control adjustments for the surgical device based on the second result.

Example 4—The surgical system of any one of Examples 1-3, wherein the surgical hub control circuit is further configured to detect that the first result is out of synchronization with the second result and replace the first result with a predetermined value.

Example 5—The surgical system of any one of Examples 1-3, wherein the surgical hub control circuit is further configured to detect that the first result is out of synchronization with the first result and resynchronize the first result with the second result based on the synchronization data.

Example 6—The surgical system of Examples 1-3, wherein the data is a first data and wherein the control adjustments are first control adjustments. The remote processing device control circuit is further configured to receive a third subset of a second data from the hub control circuit, perform a third analysis on the third subset, and transmit a third result based on the third analysis to the hub control circuit. The surgical hub control circuit is further configured to receive the second data from the surgical device, wherein the second data comprises the third subset and a fourth subset. The surgical hub control circuit is further configured to perform a fourth analysis on the fourth subset of the second data, determine a fourth result based on the fourth analysis, and receive a third result based on the third analysis from the remote processing device and synchronization data of the third result. The surgical hub control circuit is further configured to detect that the third result is out of synchronization with the fourth result, delete the third result and the fourth result, and determine second control adjustments for the surgical device based on at least one of the first result or the second result.

Example 7—The surgical system of any one of Examples 1-6, wherein the remote processing device control circuit is configured to synchronize with the hub control circuit based on the synchronization feature by adjusting a device control circuit clock to match a hub control circuit clock.

Example 8—The surgical system of any one of Examples 1-7, wherein the synchronization feature comprises a synchronization byte pattern.

Example 9—The surgical system of any one of Examples 1-8, wherein the surgical hub is located at a surgical suite in a medical facility and the remote processing device is located outside the surgical suite.

Example 10—The surgical system of any one of Examples 1-9, wherein the remote processing device is a first remote processing device, wherein the synchronization data is a first synchronization data, and wherein the surgical hub is further communicably coupled to a second remote processing device. The surgical hub control circuit is further configured to transmit the synchronization feature to the second remote processing device, transmit a third subset of the data to the second remote processing device, and receive a third result from the second remote processing device and second synchronization data of the third result. The surgical hub control circuit is further configured to assess a synchronicity of the first result, the second result, and the third result based on first synchronization data and the second synchronization data. The surgical hub control circuit is further configured to determine control adjustments for the surgical device based on the first result, the second result, and the third result.

Example 11—The surgical system of any one of Examples 1-10, wherein the surgical device is a first surgical device, wherein the surgical hub is further communicably coupled to a second surgical device, and wherein the second subset of the data is associated with the second surgical device.

Example 12—A surgical device communicably coupled to a surgical hub. The surgical device comprises a control circuit comprising a surgical device processor. The control circuit is configured to determine that an analysis of a surgical data associated with the surgical device is beyond a processing capability of the surgical device processor, transmit a synchronization feature to the surgical hub, and transmit a first subset of the surgical data to the surgical hub for a first analysis. The control circuit is further configured to perform a second analysis on a second subset of the surgical data, determine a second result based on the second analysis, and receive a first result based on the first analysis and synchronization data from the surgical hub. The control circuit is further configured to assess a synchronicity of the first result based on the synchronization data, determine control adjustments for the surgical device based on the first result and the second result, and control the surgical device according to the control adjustments.

Example 13—The surgical device of Example 12, wherein the control circuit is further configured to detect that the first result is out of synchronization based on the synchronization data, delete the first result, and determine control adjustments for the surgical device based on the second result.

Example 14—The surgical device of Example 12, wherein the control circuit is further configured to detect that the first result is out of synchronization based on the synchronization data and replace the first result with a predetermined value.

Example 15—The surgical device of Example 12, wherein the control circuit is further configured to detect that the first result is out of synchronization and resynchronize the first result with the second result based on the synchronization data.

Example 16—The surgical device of any one of Examples 12-15, wherein the synchronization feature is a synchronization signal.

Example 17—A system, comprising a remote processing device comprising a device control circuit and a surgical hub configured to communicably couple to the remote processing device. The surgical hub comprises a hub control circuit configured to transmit a first subset of a surgical data to the remote processing device for a distributed processing of the surgical data, transmit a synchronization feature associated with the first subset of the surgical data to the remote processing device, and receive, from the remote processing device, a first result of the distributed processing based on the first subset of the surgical data. The hub control circuit is further configured to receive, from the remote processing device, synchronization data. The hub control circuit is further configured to generate a second result of the distributed processing based on a second subset of the surgical data, assess a synchronicity of the first result and the second result based on the synchronization data, and determine control adjustments for the surgical device based on the first result and the second result.

Example 18—The system of Example 17, wherein hub control circuit is further configured to detect that the first result is out of synchronization based on the synchronization data, delete the first result, and determine control adjustments for the surgical device based on the second result.

Example 19—The system of Example 17, wherein the hub control circuit is further configured to detect that the first result is out of synchronization based on the synchronization data and replace the first result with a predetermined value.

Example 20—The system of Example 17, wherein the hub control circuit is further configured to detect that the first result is out of synchronization and resynchronize the first result with the second result based on the synchronization data.

Many of the surgical instrument systems described herein are motivated by an electric motor; however, the surgical instrument systems described herein can be motivated in any suitable manner. In various instances, the surgical instrument systems described herein can be motivated by a manually-operated trigger, for example. In certain instances, the motors disclosed herein may comprise a portion or portions of a robotically controlled system. Moreover, any of the end effectors and/or tool assemblies disclosed herein can be utilized with a robotic surgical instrument system. U.S. patent application Ser. No. 13/118,241, entitled SURGICAL STAPLING INSTRUMENTS WITH ROTATABLE STAPLE DEPLOYMENT ARRANGEMENTS, now U.S. Pat. No. 9,072,535, for example, discloses several examples of a robotic surgical instrument system in greater detail, and is incorporated herein by reference in its entirety.

The surgical instrument systems described herein have been described in connection with the deployment and deformation of staples; however, the embodiments described herein are not so limited. Various embodiments are envisioned which deploy fasteners other than staples, such as clamps or tacks, for example. Moreover, various embodiments are envisioned which utilize any suitable means for sealing tissue. For instance, an end effector in accordance with various embodiments can comprise electrodes configured to heat and seal the tissue. Also, for instance, an end effector in accordance with certain embodiments can apply vibrational energy to seal the tissue.

The entire disclosures of:
U.S. Pat. No. 5,403,312, entitled ELECTROSURGICAL HEMOSTATIC DEVICE, which issued on Apr. 4, 1995;
U.S. Pat. No. 7,000,818, entitled SURGICAL STAPLING INSTRUMENT HAVING SEPARATE DISTINCT CLOSING AND FIRING SYSTEMS, which issued on Feb. 21, 2006;
U.S. Pat. No. 7,422,139, entitled MOTOR-DRIVEN SURGICAL CUTTING AND FASTENING INSTRUMENT WITH TACTILE POSITION FEEDBACK, which issued on Sep. 9, 2008;
U.S. Pat. No. 7,464,849, entitled ELECTRO-MECHANICAL SURGICAL INSTRUMENT WITH CLOSURE SYSTEM AND ANVIL ALIGNMENT COMPONENTS, which issued on Dec. 16, 2008;
U.S. Pat. No. 7,670,334, entitled SURGICAL INSTRUMENT HAVING AN ARTICULATING END EFFECTOR, which issued on Mar. 2, 2010;
U.S. Pat. No. 7,753,245, entitled SURGICAL STAPLING INSTRUMENTS, which issued on Jul. 13, 2010;
U.S. Pat. No. 8,393,514, entitled SELECTIVELY ORIENTABLE IMPLANTABLE FASTENER CARTRIDGE, which issued on Mar. 12, 2013;
U.S. patent application Ser. No. 11/343,803, entitled SURGICAL INSTRUMENT HAVING RECORDING CAPABILITIES, now U.S. Pat. No. 7,845,537;
U.S. patent application Ser. No. 12/031,573, entitled SURGICAL CUTTING AND FASTENING INSTRUMENT HAVING RF ELECTRODES, filed Feb. 14, 2008;
U.S. patent application Ser. No. 12/031,873, entitled END EFFECTORS FOR A SURGICAL CUTTING AND STAPLING INSTRUMENT, filed Feb. 15, 2008, now U.S. Pat. No. 7,980,443;
U.S. patent application Ser. No. 12/235,782, entitled MOTOR-DRIVEN SURGICAL CUTTING INSTRUMENT, now U.S. Pat. No. 8,210,411;
U.S. patent application Ser. No. 12/235,972, entitled MOTORIZED SURGICAL INSTRUMENT, now U.S. Pat. No. 9,050,083.
U.S. patent application Ser. No. 12/249,117, entitled POWERED SURGICAL CUTTING AND STAPLING APPARATUS WITH MANUALLY RETRACTABLE FIRING SYSTEM, now U.S. Pat. No. 8,608,045;
U.S. patent application Ser. No. 12/647,100, entitled MOTOR-DRIVEN SURGICAL CUTTING INSTRUMENT WITH ELECTRIC ACTUATOR DIRECTIONAL CONTROL ASSEMBLY, filed Dec. 24, 2009, now U.S. Pat. No. 8,220,688;
U.S. patent application Ser. No. 12/893,461, entitled STAPLE CARTRIDGE, filed Sep. 29, 2012, now U.S. Pat. No. 8,733,613;
U.S. patent application Ser. No. 13/036,647, entitled SURGICAL STAPLING INSTRUMENT, filed Feb. 28, 2011, now U.S. Pat. No. 8,561,870;
U.S. patent application Ser. No. 13/118,241, entitled SURGICAL STAPLING INSTRUMENTS WITH ROTATABLE STAPLE DEPLOYMENT ARRANGEMENTS, now U.S. Pat. No. 9,072,535;
U.S. patent application Ser. No. 13/524,049, entitled ARTICULATABLE SURGICAL INSTRUMENT COMPRISING A FIRING DRIVE, filed on Jun. 15, 2012, now U.S. Pat. No. 9,101,358;
U.S. patent application Ser. No. 13/800,025, entitled STAPLE CARTRIDGE TISSUE THICKNESS SENSOR SYSTEM, filed on Mar. 13, 2013, now U.S. Pat. No. 9,345,481;
U.S. patent application Ser. No. 13/800,067, entitled STAPLE CARTRIDGE TISSUE THICKNESS SENSOR SYSTEM, filed on Mar. 13, 2013, now U.S. Patent Application Publication No. 2014/0263552;
U.S. Patent Application Publication No. 2007/0175955, entitled SURGICAL CUTTING AND FASTENING INSTRUMENT WITH CLOSURE TRIGGER LOCKING MECHANISM, filed Jan. 31, 2006; and
U.S. Patent Application Publication No. 2010/0264194, entitled SURGICAL STAPLING INSTRUMENT WITH AN ARTICULATABLE END EFFECTOR, filed Apr. 22, 2010, now U.S. Pat. No. 8,308,040, are hereby incorporated by reference herein.

While several forms have been illustrated and described, it is not the intention of Applicant to restrict or limit the scope of the appended claims to such detail. Numerous modifications, variations, changes, substitutions, combinations, and equivalents to those forms may be implemented and will occur to those skilled in the art without departing from the scope of the present disclosure. Moreover, the structure of each element associated with the described forms can be alternatively described as a means for providing the function performed by the element. Also, where materials are disclosed for certain components, other materials may be used. It is therefore to be understood that the foregoing description and the appended claims are intended to cover all such modifications, combinations, and variations as falling within the scope of the disclosed forms. The appended claims are intended to cover all such modifications, variations, changes, substitutions, modifications, and equivalents.

The foregoing detailed description has set forth various forms of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, and/or examples can be implemented, individually and/or collectively, by a wide range of hardware, software, firmware, or virtually any combination thereof. Those skilled in the art will recognize that some aspects of the forms disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as one or more program products in a variety of forms, and that an illustrative form of the subject matter described herein applies regardless of the particular type of signal bearing medium used to actually carry out the distribution.

Instructions used to program logic to perform various disclosed aspects can be stored within a memory in the system, such as dynamic random access memory (DRAM), cache, flash memory, or other storage. Furthermore, the instructions can be distributed via a network or by way of other computer readable media. Thus a machine-readable medium may include any mechanism for storing or transmitting information in a form readable by a machine (e.g., a computer), but is not limited to, floppy diskettes, optical disks, compact disc, read-only memory (CD-ROMs), and magneto-optical disks, read-only memory (ROMs), random access memory (RAM), erasable programmable read-only memory (EPROM), electrically erasable programmable read-only memory (EEPROM), magnetic or optical cards, flash memory, or a tangible, machine-readable storage used in the transmission of information over the Internet via electrical, optical, acoustical or other forms of propagated signals (e.g., carrier waves, infrared signals, digital signals, etc.). Accordingly, the non-transitory computer-readable medium includes any type of tangible machine-readable medium suitable for storing or transmitting electronic instructions or information in a form readable by a machine (e.g., a computer).

As used in any aspect herein, the term “control circuit” may refer to, for example, hardwired circuitry, programmable circuitry (e.g., a computer processor including one or more individual instruction processing cores, processing unit, processor, microcontroller, microcontroller unit, controller, digital signal processor (DSP), programmable logic device (PLD), programmable logic array (PLA), or field programmable gate array (FPGA)), state machine circuitry, firmware that stores instructions executed by programmable circuitry, and any combination thereof. The control circuit may, collectively or individually, be embodied as circuitry that forms part of a larger system, for example, an integrated circuit (IC), an application-specific integrated circuit (ASIC), a system on-chip (SoC), desktop computers, laptop computers, tablet computers, servers, smart phones, etc. Accordingly, as used herein “control circuit” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

As used in one or more aspects of the present disclosure, a microcontroller may generally comprise a memory and a microprocessor (“processor”) operationally coupled to the memory. The processor may control a motor driver circuit generally utilized to control the position and velocity of a motor, for example. In certain instances, the processor can signal the motor driver to stop and/or disable the motor, for example. In certain instances, the microcontroller may be an LM 4F230H5QR, available from Texas Instruments, for example. In at least one example, the Texas Instruments LM4F230H5QR is an ARM Cortex-M4F Processor Core comprising on-chip memory of 256 KB single-cycle flash memory, or other non-volatile memory, up to 40 MHz, a prefetch buffer to improve performance above 40 MHz, a 32 KB single-cycle serial random access memory (SRAM), internal read-only memory (ROM) loaded with StellarisWare® software, 2 KB electrically erasable programmable read-only memory (EEPROM), one or more pulse width modulation (PWM) modules, one or more quadrature encoder inputs (QEI) analog, one or more 12-bit Analog-to-Digital Converters (ADC) with 12 analog input channels, among other features that are readily available for the product datasheet.

It should be understood that the term processor as used herein includes any suitable microprocessor, or other basic computing device that incorporates the functions of a computer's central processing unit (CPU) on an integrated circuit or at most a few integrated circuits. The processor is a multipurpose, programmable device that accepts digital data as input, processes it according to instructions stored in its memory, and provides results as output. It is an example of sequential digital logic, as it has internal memory. Processors operate on numbers and symbols represented in the binary numeral system.

In at least one instance, the processor may be any single core or multicore processor such as those known under the trade name ARM Cortex by Texas Instruments. Nevertheless, other suitable substitutes for microcontrollers and safety processor may be employed, without limitation.

As used in any aspect herein, the term “logic” may refer to an app, software, firmware and/or circuitry configured to perform any of the aforementioned operations. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in memory devices.

As used in any aspect herein, the terms “component,” “system,” “module” and the like can refer to a computer-related entity, either hardware, a combination of hardware and software, software, or software in execution.

As used in any aspect herein, an “algorithm” refers to a self-consistent sequence of steps leading to a desired result, where a “step” refers to a manipulation of physical quantities and/or logic states which may, though need not necessarily, take the form of electrical or magnetic signals capable of being stored, transferred, combined, compared, and otherwise manipulated. It is common usage to refer to these signals as bits, values, elements, symbols, characters, terms, numbers, or the like. These and similar terms may be associated with the appropriate physical quantities and are merely convenient labels applied to these quantities and/or states.

Various instruments, tools, hubs, devices and/or systems, in accordance with the present disclosure, may be capable of communicating with each other using a selected packet switched network communications protocol. One example communications protocol may include an Ethernet communications protocol which may be capable permitting communication using a Transmission Control Protocol/Internet Protocol (TCP/IP). The Ethernet protocol may comply or be compatible with the Ethernet standard published by the Institute of Electrical and Electronics Engineers (IEEE) titled “IEEE 802.3 Standard”, published in December, 2008 and/or later versions of this standard. Alternatively or additionally, the communication devices may be capable of communicating with each other using an X.25 communications protocol. The X.25 communications protocol may comply or be compatible with a standard promulgated by the International Telecommunication Union-Telecommunication Standardization Sector (ITU-T). Alternatively or additionally, the communication devices may be capable of communicating with each other using a frame relay communications protocol. The frame relay communications protocol may comply or be compatible with a standard promulgated by Consultative Committee for International Telegraph and Telephone (CCITT) and/or the American National Standards Institute (ANSI). Alternatively or additionally, transceivers may be capable of communicating with each other using an Asynchronous Transfer Mode (ATM) communications protocol. The ATM communications protocol may comply or be compatible with an ATM standard published by the ATM Forum titled “ATM-MPLS Network Interworking 2.0” published August 2001, and/or later versions of this standard. Of course, different and/or after-developed connection-oriented network communication protocols are equally contemplated herein.

One or more motor assemblies, as described herein, employ one or more electric motors. In various forms, the electric motors may be a DC brushed driving motor, for example. In other arrangements, the motor may include a brushless motor, a cordless motor, a synchronous motor, a stepper motor, or any other suitable electric motor. The electric motors may be powered by a power source that in one form may comprise a removable power pack. Batteries may each comprise, for example, a Lithium Ion (“LI”) or other suitable battery. The electric motors can include rotatable shafts that operably interface with gear reducer assemblies, for example. In certain instances, a voltage polarity provided by the power source can operate an electric motor in a clockwise direction wherein the voltage polarity applied to the electric motor by the battery can be reversed in order to operate the electric motor in a counter-clockwise direction. In various aspects, a microcontroller controls the electric motor through a motor driver via a pulse width modulated control signal. The motor driver can be configured to adjust the speed of the electric motor either in clockwise or counter-clockwise direction. The motor driver is also configured to switch between a plurality of operational modes which include an electronic motor braking mode, a constant speed mode, an electronic clutching mode, and a controlled current activation mode. In electronic braking mode, two terminal of the drive motor are shorted and the generated back EMF counteracts the rotation of the electric motor allowing for faster stopping and greater positional precision.

As used in any aspect herein, a wireless transmission such as, for example, a wireless communication or a wireless transfer of a data signal can be achieved, by a device including one or more transceivers. The transceivers may include, but are not limited to cellular modems, wireless mesh network transceivers, Wi-Fi® transceivers, low power wide area (LPWA) transceivers, and/or near field communications transceivers (NFC). The device may include or may be configured to communicate with a mobile telephone, a sensor system (e.g., environmental, position, motion, etc.) and/or a sensor network (wired and/or wireless), a computing system (e.g., a server, a workstation computer, a desktop computer, a laptop computer, a tablet computer (e.g., iPad®, GalaxyTab® and the like), an ultraportable computer, an ultramobile computer, a netbook computer and/or a subnotebook computer; etc. In at least one aspect of the present disclosure, one of the devices may be a coordinator node.

The transceivers may be configured to receive serial transmit data via respective universal asynchronous receiver-transmitters (UARTs) from a processor to modulate the serial transmit data onto an RF carrier to produce a transmit RF signal and to transmit the transmit RF signal via respective antennas. The transceiver(s) can be further configured to receive a receive RF signal via respective antennas that includes an RF carrier modulated with serial receive data, to demodulate the receive RF signal to extract the serial receive data and to provide the serial receive data to respective UARTs for provision to the processor. Each RF signal has an associated carrier frequency and an associated channel bandwidth. The channel bandwidth is associated with the carrier frequency, the transmit data and/or the receive data. Each RF carrier frequency and channel bandwidth is related to the operating frequency range(s) of the transceiver(s). Each channel bandwidth is further related to the wireless communication standard and/or protocol with which the transceiver(s) may comply. In other words, each transceiver may correspond to an implementation of a selected wireless communication standard and/or protocol, e.g., IEEE 802.11 a/b/g/n for Wi-Fi® and/or IEEE 802.15.4 for wireless mesh networks using Zigbee routing.

Unless specifically stated otherwise as apparent from the foregoing disclosure, it is appreciated that, throughout the foregoing disclosure, discussions using terms such as “processing,” “computing,” “calculating,” “determining,” “displaying,” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

One or more components may be referred to herein as “configured to,” “configurable to,” “operable/operative to,” “adapted/adaptable,” “able to,” “conformable/conformed to,” etc. Those skilled in the art will recognize that “configured to” can generally encompass active-state components and/or inactive-state components and/or standby-state components, unless context requires otherwise.

The terms “proximal” and “distal” are used herein with reference to a clinician manipulating the handle portion of the surgical instrument. The term “proximal” refers to the portion closest to the clinician and the term “distal” refers to the portion located away from the clinician. It will be further appreciated that, for convenience and clarity, spatial terms such as “vertical”, “horizontal”, “up”, and “down” may be used herein with respect to the drawings. However, surgical instruments are used in many orientations and positions, and these terms are not intended to be limiting and/or absolute.

Those skilled in the art will recognize that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flow diagrams are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

It is worthy to note that any reference to “one aspect,” “an aspect,” “an exemplification,” “one exemplification,” and the like means that a particular feature, structure, or characteristic described in connection with the aspect is included in at least one aspect. Thus, appearances of the phrases “in one aspect,” “in an aspect,” “in an exemplification,” and “in one exemplification” in various places throughout the specification are not necessarily all referring to the same aspect. Furthermore, the particular features, structures or characteristics may be combined in any suitable manner in one or more aspects.

In this specification, unless otherwise indicated, terms “about” or “approximately” as used in the present disclosure, unless otherwise specified, means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

In this specification, unless otherwise indicated, all numerical parameters are to be understood as being prefaced and modified in all instances by the term “about,” in which the numerical parameters possess the inherent variability characteristic of the underlying measurement techniques used to determine the numerical value of the parameter. At the very least, and not as an attempt to limit the application of the doctrine of equivalents to the scope of the claims, each numerical parameter described herein should at least be construed in light of the number of reported significant digits and by applying ordinary rounding techniques.

Any numerical range recited herein includes all sub-ranges subsumed within the recited range. For example, a range of “1 to 10” includes all sub-ranges between (and including) the recited minimum value of 1 and the recited maximum value of 10, that is, having a minimum value equal to or greater than 1 and a maximum value equal to or less than 10. Also, all ranges recited herein are inclusive of the end points of the recited ranges. For example, a range of “1 to 10” includes the end points 1 and 10. Any maximum numerical limitation recited in this specification is intended to include all lower numerical limitations subsumed therein, and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited. All such ranges are inherently described in this specification.

Any patent application, patent, non-patent publication, or other disclosure material referred to in this specification and/or listed in any Application Data Sheet is incorporated by reference herein, to the extent that the incorporated materials is not inconsistent herewith. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

In summary, numerous benefits have been described which result from employing the concepts described herein. The foregoing description of the one or more forms has been presented for purposes of illustration and description. It is not intended to be exhaustive or limiting to the precise form disclosed. Modifications or variations are possible in light of the above teachings. The one or more forms were chosen and described in order to illustrate principles and practical application to thereby enable one of ordinary skill in the art to utilize the various forms and with various modifications as are suited to the particular use contemplated. It is intended that the claims submitted herewith define the overall scope.

What is claimed is:

1. A surgical system for use with a surgical device, the surgical system comprising:
a remote processing device comprising a device control circuit; and
a surgical hub configured to communicably couple to the remote processing device and the surgical device, the surgical hub comprising a hub control circuit, wherein the hub control circuit and the device control circuit perform distributed processing,
wherein the remote processing device control circuit is configured to:
receive a first subset of data associated with the surgical device from the hub control circuit;
receive a synchronization feature associated with the data from the hub control circuit;
perform a first analysis on the first subset of the data; and
transmit a first result based on the first analysis to the hub control circuit, wherein the first result comprises synchronization data of the remote processing device based on the synchronization feature; and
wherein the surgical hub control circuit is configured to:
transmit the synchronization feature to the remote processing device;
transmit the first subset of the data to the remote processing device;
perform a second analysis on a second subset of the data;
determine a second result based on the second analysis;
receive the first result from the remote processing device and synchronization data of the first result;
assess a synchronicity of the first result and the second result based on the synchronization data; and
determine control adjustments for the surgical device based on at least one of the first result or the second result.

2. The surgical system of claim 1, wherein the synchronization feature is a synchronization signal.

3. The surgical system of claim 1, wherein the surgical hub control circuit is further configured to:
detect that the first result is out of synchronization with the second result;
delete the first result; and
determine the control adjustments for the surgical device based on the second result.

4. The surgical system of claim 1, wherein the surgical hub control circuit is further configured to:
detect that the first result is out of synchronization with the second result; and
replace the first result with a predetermined value.

5. The surgical system of claim 1, wherein the surgical hub control circuit is further configured to:
detect that the first result is out of synchronization with the first result; and
resynchronize the first result with the second result based on the synchronization data.

6. The surgical system of claim 1, wherein the data is a first data; wherein the control adjustments are first control adjustments; wherein the remote processing device control circuit is further configured to:
receive a third subset of a second data from the hub control circuit;
perform a third analysis on the third subset; and
transmit a third result based on the third analysis to the hub control circuit; and
wherein the surgical hub control circuit is further configured to:
receive the second data from the surgical device, wherein the second data comprises the third subset and a fourth subset;
perform a fourth analysis on the fourth subset of the second data;
determine a fourth result based on the fourth analysis;
receive a third result based on the third analysis from the remote processing device and synchronization data of the third result;
detect that the third result is out of synchronization with the fourth result;
delete the third result and the fourth result; and
determine second control adjustments for the surgical device based on at least one of the first result or the second result.

7. The surgical system of claim 1, wherein the remote processing device control circuit is configured to synchronize with the hub control circuit based on the synchronization feature by adjusting a device control circuit clock to match a hub control circuit clock.

8. The surgical system of claim 1, wherein the synchronization feature comprises a synchronization byte pattern.

9. The surgical system of claim 1, wherein the surgical hub is located at a surgical suite in a medical facility and the remote processing device is located outside the surgical suite.

10. The surgical system of claim 1, wherein the remote processing device is a first remote processing device, wherein the synchronization data is a first synchronization data, wherein the surgical hub is further communicably coupled to a second remote processing device, and wherein the surgical hub control circuit is further configured to:
transmit the synchronization feature to the second remote processing device;
transmit a third subset of the data to the second remote processing device;
receive a third result from the second remote processing device and second synchronization data of the third result;
assess a synchronicity of the first result, the second result, and the third result based on first synchronization data and the second synchronization data; and
determine control adjustments for the surgical device based on the first result, the second result, and the third result.

11. The surgical system of claim 1, wherein the surgical device is a first surgical device, wherein the surgical hub is further communicably coupled to a second surgical device, and wherein the second subset of the data is associated with the second surgical device.

12. A surgical device communicably coupled to a surgical hub, the surgical device comprising:
a control circuit comprising a surgical device processor, wherein the control circuit is configured to:
determine that an analysis of a surgical data associated with the surgical device is beyond a processing capability of the surgical device processor;
transmit a synchronization feature to the surgical hub;
transmit a first subset of the surgical data to the surgical hub for a first analysis;
perform a second analysis on a second subset of the surgical data;
determine a second result based on the second analysis;
receive a first result based on the first analysis and synchronization data from the surgical hub;
assess a synchronicity of the first result based on the synchronization data;
determine control adjustments for the surgical device based on the first result and the second result; and
control the surgical device according to the control adjustments.

13. The surgical device of claim 12, wherein the control circuit is further configured to:
detect that the first result is out of synchronization based on the synchronization data;
delete the first result; and
determine control adjustments for the surgical device based on the second result.

14. The surgical device of claim 12, wherein the control circuit is further configured to:
detect that the first result is out of synchronization based on the synchronization data; and
replace the first result with a predetermined value.

15. The surgical device of claim 12, wherein the control circuit is further configured to:
detect that the first result is out of synchronization; and
resynchronize the first result with the second result based on the synchronization data.

16. The surgical device of claim 12, wherein the synchronization feature is a synchronization signal.

17. A surgical system, comprising:
a remote processing device comprising a device control circuit; and
a surgical hub configured to communicably couple to the remote processing device, the surgical hub comprising a hub control circuit configured to:
transmit a first subset of a surgical data to the remote processing device for a distributed processing of the surgical data;
transmit a synchronization feature associated with the first subset of the surgical data to the remote processing device;
receive, from the remote processing device, a first result of the distributed processing based on the first subset of the surgical data;
receive, from the remote processing device, synchronization data;
generate a second result of the distributed processing based on a second subset of the surgical data;
assess a synchronicity of the first result and the second result based on the synchronization data; and
determine control adjustments for the surgical device based on the first result and the second result.

18. The surgical system of claim 17, wherein hub control circuit is further configured to:
detect that the first result is out of synchronization based on the synchronization data;
delete the first result; and
determine control adjustments for the surgical device based on the second result.

19. The surgical system of claim 17, wherein the hub control circuit is further configured to:
detect that the first result is out of synchronization based on the synchronization data; and
replace the first result with a predetermined value.

20. The surgical system of claim 17, wherein the hub control circuit is further configured to:
detect that the first result is out of synchronization; and
resynchronize the first result with the second result based on the synchronization data.
